(19) United States
(12) Patent Application Publication
(10) Pub. No.: US 2012/0157452 A1
(43) Pub. Date: Jun. 21, 2012

(54) 1H-PYRAZOLO[3,4-B] PYRIDINE COMPOUNDS FOR INHIBITING RAF KINASE

(75) Inventors: Stefan Gradl, South San Francisco, CA (US); Ellen Laird, Boulder, CO (US); David Moreno, Boulder, CO (US); Li Ren, Boulder, CO (US); Steven Mark Wenglowsky, Boulder, CO (US)

(73) Assignees: GENENTECH, INC., South San Francisco, CA (US); ARRAY BIOPHARMA INC., Boulder, CO (US)

(21) Appl. No.: 13/393,076

(22) PCT Filed: Aug. 27, 2010
(86) PCT No.: PCT/US2010/047013

§ 371 Date: Feb. 28, 2012

Related U.S. Application Data
(60) Provisional application No. 61/238,104, filed on Aug. 28, 2009.

(51) Int. Cl. A61K 31/437; A61P 35/00; A61P 13/12; A61K 31/4709; A61K 31/506; A61K 31/497; A61K 31/5377; C07D 471/04; A61K 31/444

(52) U.S. Cl. 514/234.2; 546/119; 514/303; 544/333; 514/256; 544/405; 514/255.05; 544/127

(57) ABSTRACT

Compounds of Formula I are useful for inhibition of Raf kinases. Methods of using compounds of Formula I and stereoisomers, tautomers and pharmaceutically acceptable salts thereof, for in vitro, in situ, and in vivo diagnosis, prevention or treatment of such disorders in mammalian cells, or associated pathological conditions are disclosed. [FORMULA I]

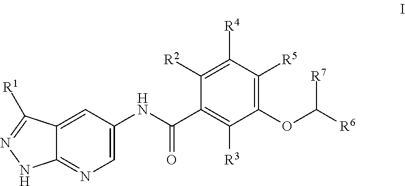

### PRIORITY OF INVENTION

This application claims priority under 35 U.S.C. 119(e) from U.S. Provisional Patent Application No. 61/238,104, filed 28 Aug. 2009, the content of which is incorporated herein in its entirety.

### BACKGROUND OF THE INVENTION

1. Field of the Invention

Compounds, pharmaceutical compositions comprising the compounds, a process for making the compounds and the use of the compounds in therapy are provided herein. More particularly, certain substituted 1H-pyrazolo[3,4-b]pyridine compounds useful for inhibiting Raf kinase and for treating disorders mediated thereby are disclosed herein.

2. Description of the State of the Art

The Raf/MEK/ERK pathway is critical for cell survival, growth, proliferation and tumorigenesis. Li, Nanxin, et al. “B-Raf kinase inhibitors for cancer treatment.” Current Opinion in Investigational Drugs. Vol. 8, No. 6 (2007): 452-456. Raf kinases exist as three isoforms, A-Raf, B-Raf and C-Raf. Among the three isoforms, studies have shown that B-Raf functions as the primary MEK activator. B-Raf is one of the most frequently mutated genes in human cancers. B-Raf kinase represents an excellent target for anticancer therapy based on preclinical target validation, epidemiology and drugability.

Small molecule inhibitors of B-Raf are being developed for anticancer therapy. Nexavar® (sorafenib tosylate) is a multikinase inhibitor, which includes inhibition of B-Raf, and is approved for the treatment of patients with advanced renal cell carcinoma and unresectable hepatocellular carcinoma. Other Raf inhibitors have also been disclosed or have entered clinical trials, for example GSK-2118436, RAF-265, PLX-4032, PLX3603 and XL-281. Other B-Raf inhibitors are also known, see for example, U.S. Patent Application Publication 2006/0189627, U.S. Patent Application Publication 2006/0281751, U.S. Patent Application Publication 2007/0049603, U.S. Patent Application Publication 2009/0176809, International Patent Application Publication WO 2007/002325, International Patent Application Publication WO 2007/002433, International Patent Application Publication WO 2008/028141, International Patent Application Publication WO 2008/079903, International Patent Application Publication WO 2008/079906 and International Patent Application Publication WO 2009/012283.

Pyrazolopyridines are known, see for example, International Patent Application Publication WO 03/068773 and International Patent Application Publication WO 2007/013896.

Kinase inhibitors are known, see for example, International Patent Application Publication WO 2006/066913, International Patent Application Publication WO 2008/028617 and International Patent Application Publication WO 2008/079909.

### SUMMARY OF THE INVENTION

Compounds that are inhibitors of Raf kinases, particularly B-Raf inhibitors, are described herein. Certain hyperproliferative disorders are characterized by the over activation of Raf kinase function, for example by mutations or over expression of the protein. Accordingly, the compounds are useful in the treatment of hyperproliferative disorders, such as cancer.

More specifically, one aspect provides compounds of Formula I:

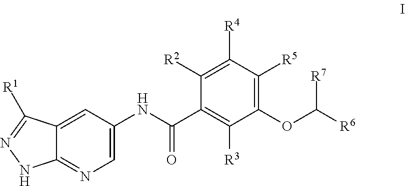

and stereoisomers, tautomers and pharmaceutically acceptable salts thereof, wherein R¹, R², R³, R⁴, R⁵, R⁶ and R⁷ are as defined herein.

Another aspect provides methods of preventing or treating a disease or disorder modulated by B-Raf, comprising administering to a mammal in need of such treatment an effective amount of a compound of Formula I, a stereoisomer, tautomer or pharmaceutically acceptable salt thereof. Examples of such diseases and disorders include, but are not limited to, hyperproliferative disorders (such as cancer, including melanoma and other cancers of the skin), neurodegeneration, cardiac hypertrophy, pain, migraine and neurotraumatic disease.

Another aspect provides methods of preventing or treating cancer, comprising administering to a mammal in need of such treatment an effective amount of a compound of Formula I, a stereoisomer, tautomer or pharmaceutically acceptable salt thereof, alone or in combination with one or more additional compounds having anti-cancer properties.

Another aspect provides a method of treating a hyperproliferative disease in a mammal comprising administering a therapeutically effective amount of a compound of Formula I, a stereoisomer, tautomer or pharmaceutically acceptable salt thereof to the mammal.

Another aspect provides methods of preventing or treating kidney disease, comprising administering to a mammal in need of such treatment an effective amount of a compound of Formula I, a stereoisomer, tautomer or pharmaceutically acceptable salt thereof, alone or in combination with one or more additional compounds. Another aspect provides methods of preventing or treating polycystic kidney disease, comprising administering to a mammal in need of such treatment an effective amount of a compound of Formula I, a stereoisomer or pharmaceutically acceptable salt thereof, alone or in combination with one or more additional compounds.

Another aspect provides the use of a compound of Formula I, a stereoisomer, tautomer or pharmaceutically acceptable salt thereof in the manufacture of a medicament for the treatment of a hyperproliferative disease.

Another aspect provides the compounds of Formula I, a stereoisomer, tautomer or pharmaceutically acceptable salt thereof for use in therapy.

Another aspect provides the compounds of Formula I, a stereoisomer, tautomer or pharmaceutically acceptable salt thereof for use in the treatment of a hyperproliferative disease. In a further embodiment, the hyperproliferative disease may be cancer (or still further, a specific cancer as defined herein).

Another aspect provides the compounds of Formula I, a stereoisomer, tautomer or pharmaceutically acceptable salt thereof for use in the treatment of a kidney disease. In a further embodiment, the kidney disease may be polycystic kidney disease.

Another aspect provides the use of a compound of Formula I, a stereoisomer, tautomer or pharmaceutically acceptable salt thereof in the manufacture of a medicament for the treatment of a hyperproliferative disease. In a further embodiment, the hyperproliferative disease may be cancer (or still further, a specific cancer as defined herein).

Another aspect provides the use of a compound of Formula I, a stereoisomer, tautomer or pharmaceutically acceptable salt thereof in the manufacture of a medicament for the treatment of a kidney disease. In a further embodiment, the kidney disease may be polycystic kidney disease.

Another aspect provides the use of a compound of Formula I, a stereoisomer, tautomer or pharmaceutically acceptable salt thereof in the manufacture of a medicament, for use as a B-Raf inhibitor in the treatment of a patient undergoing cancer therapy.

Another aspect provides the use of a compound of Formula I, a stereoisomer, tautomer or pharmaceutically acceptable salt thereof in the manufacture of a medicament, for use as a B-Raf inhibitor in the treatment of a patient undergoing polycystic kidney disease therapy.

Another aspect provides a pharmaceutical composition comprising a compound of Formula I, a stereoisomer, tautomer or pharmaceutically acceptable salt thereof for use in the treatment of a hyperproliferative disease.

Another aspect provides a pharmaceutical composition comprising a compound of Formula I, a stereoisomer, tautomer or pharmaceutically acceptable salt thereof for use in the treatment of cancer.

Another aspect provides a pharmaceutical composition comprising a compound of Formula I, a stereoisomer, tautomer or pharmaceutically acceptable salt thereof for use in the treatment of polycystic kidney disease.

Another aspect provides a pharmaceutical composition comprising a compound of Formula I, a stereoisomer, tautomer or pharmaceutically acceptable salt thereof and a pharmaceutically acceptable carrier or excipient.

Another aspect provides intermediates for preparing compounds of Formula I. Certain compounds of Formula I may be used as intermediates for other compounds of Formula I.

Another aspect includes processes for preparing, methods of separation, and methods of purification of the compounds described herein.

### BRIEF DESCRIPTION OF THE FIGURES

FIG. 1 shows a TGI experiment in nude mice with subcutaneous LOX xenografts.

FIG. 2 shows a TGI experiment in nude mice with subcutaneous LOX xenografts.

### DETAILED DESCRIPTION OF THE INVENTION

Reference will now be made in detail to certain embodiments, examples of which are illustrated in the accompanying structures and formulas. While enumerated embodiments will be described, it will be understood that they are not intended to limit the invention to those embodiments. On the contrary, the invention is intended to cover all alternatives, modifications, and equivalents, which may be included within the scope of the present invention as defined by the claims. One skilled in the art will recognize many methods and materials similar or equivalent to those described herein, which could be used in the practice of the present invention. The present invention is in no way limited to the methods and materials described. In the event that one or more of the incorporated literature and similar materials differs from or contradicts this application, including but not limited to defined terms, term usage, described techniques, or the like, this application controls.

### DEFINITIONS

The term “alkyl” includes linear or branched-chain radicals of carbon atoms. In one example, the alkyl radical may be one to six carbon atoms (C₁-C₆). In other examples, the alkyl radical may be C₁-C₅, C₁-C₄ or C₁-C₃. Some alkyl moieties have been abbreviated, for example, methyl (“Me”), ethyl (“Et”), propyl (“Pr”) and butyl (“Bu”), and further abbreviations are used to designate specific isomers of compounds, for example, 1-propyl or n-propyl (“n-Pr”), 2-propyl or isopropyl (“i-Pr”), 1-butyl or n-butyl (“n-Bu”), 2-methyl-1-propyl or isobutyl (“i-Bu”), 1-methylpropyl or s-butyl (“s-Bu”), 1,1-dimethylethyl or t-butyl (“t-Bu”) and the like. The abbreviations are sometimes used in conjunction with elemental abbreviations and chemical structures, for example, methanol (“MeOH”) or ethanol (“EtOH”).

Additional abbreviations used throughout the application may include, for example, benzyl (“Bn”), phenyl (“Ph”) and acetate (“Ac”).

The term “alkenyl” includes linear or branched-chain monovalent hydrocarbon radical with at least one site of unsaturation, i.e., a carbon-carbon double bond, wherein the alkenyl radical may be optionally substituted independently with one or more substituents described herein, and includes radicals having “cis” and “trans” orientations, or alternatively, “E” and “Z” orientations. In one example, the alkenyl radical may be two to six carbon atoms (C₂-C₆). In other examples, the alkenyl radical may be C₂-C₅, C₂-C₄ or C₂-C₃.

The term “alkynyl” includes linear or branched-chain monovalent hydrocarbon radical with at least one site of unsaturation, i.e., a carbon-carbon triple bond, wherein the alkynyl radical may be optionally substituted independently with one or more substituents described herein. In one example, the alkynyl radical may be two to six carbon atoms (C₂-C₆). In other examples, the alkynyl radical may be C₂-C₅, C₂-C₄ or C₂-C₃.

The term “alkoxy” refers to a radical of the formula —O-(alkyl), wherein the alkyl may be substituted.

The term “cycloalkyl” refers to a non-aromatic, saturated or partially unsaturated hydrocarbon ring group, wherein the cycloalkyl group may be optionally substituted independently with one or more substituents described herein. In one example, the cycloalkyl group may be 3 to 6 carbon atoms (C₃-C₆). In other examples, cycloalkyl may be C₅-C₆, C₃-C₄ or C₃-C₅.

The terms “heterocycle”, “heterocyclic” and “heterocyclyl” include saturated or a partially unsaturated four to seven membered rings containing one, two or three heteroatoms selected from the group consisting of oxygen, nitrogen and sulfur, with the remaining atoms being carbon. In one example, the heterocyclic may be a 3 to 6 membered ring. In other examples, the heterocyclic may be a 4 to 6 membered ring or a 5 to 6 membered ring.

The term “heteroaryl” includes five to six membered aromatic rings containing one, two or three heteroatoms selected from the group consisting of oxygen, nitrogen and sulfur, with the remaining atoms being carbon. In one example, the heteroaryl may be a 5 to 6 membered ring.

The term “halogen” refers to F, Cl, Br or I.

The terms “treat” or “treatment” refer to therapeutic, prophylactic, palliative or preventative measures. Beneficial or desired clinical results include, but are not limited to, alleviation of symptoms, diminishment of extent of disease, stabilized (i.e., not worsening) state of disease, delay or slowing of disease progression, amelioration or palliation of the disease state, and remission (whether partial or total), whether detectable or undetectable. “Treatment” can also mean prolonging survival as compared to expected survival if not receiving treatment. Those in need of treatment include those already with the condition or disorder, as well as those prone to have the condition or disorder or those in which the condition or disorder is to be prevented.

The phrases “therapeutically effective amount” or “effective amount” mean an amount of a compound of Formula I that, when administered to a mammal in need of such treatment, sufficient to (i) treat or prevent the particular disease, condition, or disorder, (ii) attenuate, ameliorate, or eliminate one or more symptoms of the particular disease, condition, or disorder, or (iii) prevent or delay the onset of one or more symptoms of the particular disease, condition, or disorder described herein. The amount of a compound that will correspond to such an amount will vary depending upon factors such as the particular compound, disease condition and its severity, the identity (e.g., weight) of the mammal in need of treatment, but can nevertheless be routinely determined by one skilled in the art.

The terms “cancer” and “cancerous” refer to or describe the physiological condition in mammals that is typically characterized by abnormal or unregulated cell growth. A “tumor” comprises one or more cancerous cells. Examples of cancer include, but are not limited to, carcinoma, lymphoma, blastoma, sarcoma, and leukemia or lymphoid malignancies. More particular examples of such cancers include squamous cell cancer (e.g., epithelial squamous cell cancer), lung cancer including small-cell lung cancer, non-small cell lung cancer (“NSCLC”), adenocarcinoma of the lung and squamous carcinoma of the lung, cancer of the peritoneum, hepatocellular cancer, gastric or stomach cancer including gastrointestinal cancer, pancreatic cancer, glioblastoma, cervical cancer, ovarian cancer, liver cancer, bladder cancer, hepatoma, breast cancer, colon cancer, rectal cancer, colorectal cancer, endometrial or uterine carcinoma, salivary gland carcinoma, kidney or renal cancer, prostate cancer, vulval cancer, thyroid cancer, hepatic carcinoma, anal carcinoma, penile carcinoma, skin cancer, including melanoma, as well as head and neck cancer.

The phrase “pharmaceutically acceptable” indicates that the substance or composition is compatible chemically and/or toxicologically, with the other ingredients comprising a formulation, and/or the mammal being treated therewith.

The phrase “pharmaceutically acceptable salt,” as used herein, refers to pharmaceutically acceptable organic or inorganic salts of a compound described herein.

The compounds described herein also include other salts of such compounds that are not necessarily pharmaceutically acceptable salts, and which may be useful as intermediates for preparing and/or purifying compounds described herein and/or for separating enantiomers of compounds described herein.

The term “mammal” means a warm-blooded animal that has or is at risk of developing a disease described herein and includes, but is not limited to, guinea pigs, dogs, cats, rats, mice, hamsters, and primates, including humans.

B-Raf Inhibitor Compounds

Provided herein are compounds, and pharmaceutical formulations thereof, that are potentially useful in the treatment of diseases, conditions and/or disorders modulated by B-Raf.

One embodiment provides compounds of Formula I:

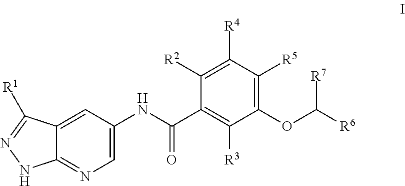

and stereoisomers, tautomers and pharmaceutically acceptable salts thereof, wherein:

R¹ is selected from:
hydrogen,
halogen,
CN,
NR^{a}R^{b},
OR^{c},
SR^{d},
phenyl optionally substituted with one to three R^{e} groups,
a 5-6 membered heteroaryl optionally substituted with C₁-C₄ alkyl,
a saturated or partially unsaturated C₃-C₆ cycloalkyl optionally substituted with halogen or C₁-C₄ alkyl,
a saturated or partially unsaturated 4-6 membered heterocyclyl optionally substituted with C₁-C₄ alkyl,
C₂-C₆ alkynyl optionally substituted with halogen, OR^{c} or NR^{a}R^{b},
C₂-C₆ alkenyl optionally substituted with halogen, OR^{c} or NR^{a}R^{b}, or
C₁-C₆ alkyl optionally substituted with one to three R^{f} groups;

R² and R³ are independently selected from hydrogen, halogen, C₁-C₃ alkyl and C₁-C₃ alkoxy;

R⁴ and R⁵ are independently selected from hydrogen, halogen or C₁-C₃ alkyl;

R⁶ is selected from phenyl, a 5-6 membered heteroaryl, a 9-10 membered bicyclic heterocyclyl or a 9-10 membered bicyclic heteroaryl, wherein the phenyl, heteroaryls and heterocyclyl are optionally substituted with one, two or three R^{g} groups;

R⁷ is hydrogen or methyl;

R^{a} and R^{b} are independently selected from hydrogen, phenyl and C₁-C₄ alkyl optionally substituted with oxo;

R^{c} is selected from a 4-6 membered heterocyclyl and C₁-C₆ alkyl optionally substituted with halogen, OH, OCH₃, C₃-C₆ cycloalkyl, a 4-6 membered heterocyclyl or NR^{a}R^{b};

R^{d} is C₁-C₆ alkyl;

each R^{e} is independently selected from halogen, CF₃, C₁-C₄ alkyl or C₁-C₄ alkoxy, wherein the alkyl or alkoxy are optionally substituted with OH, NR^{a}R^{b} or a 5-6 membered heterocyclyl optionally substituted with C₁-C₃ alkyl;

each R^{f} is independently selected from halogen, OH, OCH₃, oxo, NR^{a}R^{b}, or C₃-C₆ cycloalkyl; and

each R^{g} is selected from halogen, CN, SO₂CH₃, C₁-C₃ alkyl, C₁-C₃ alkoxy, or C₃-C₆ cycloalkyl, wherein the alkyl is optionally substituted with halogen or a 3-6 membered heterocyclyl.

In certain embodiments, each R^{g} is selected from halogen, CN, SO₂CH₃, C₁-C₃ alkyl, C₁-C₃ alkoxy, wherein the alkyl is optionally substituted with halogen or a 3-6 membered heterocyclyl.

In certain embodiments, R¹ is selected from NR^{a}R^{b}, OR^{c}, SR^{d}, a 5-6 membered heteroaryl, C₃-C₆ cycloalkyl and C₁-C₆ alkyl optionally substituted with one to three R^{f} groups. In certain embodiments, R^{a} and R^{b} are independently selected from hydrogen and C₁-C₄ alkyl optionally substituted with alkyl. In certain embodiments, R^{c} is C₁-C₆ alkyl optionally substituted with halogen, OH, OCH₃ or NR^{a}R^{b}. In certain embodiments, R^{d} is C₁-C₆ alkyl. In certain embodiments, each R^{f} is halogen.

In certain embodiments, R¹ is selected from NR^{a}R^{b}, SR^{d}, a 5-6 membered heteroaryl, C₃-C₆ cycloalkyl and C₁-C₆ alkyl optionally substituted with one to three R^{f} groups. In certain embodiments, R^{a} and R^{b} are independently selected from hydrogen and C₁-C₄ alkyl. In certain embodiments, R^{c} is C₁-C₆ alkyl optionally substituted with halogen, OH, OCH₃ or NR^{a}R^{b}. In certain embodiments, R^{d} is C₁-C₆ alkyl. In certain embodiments, each R^{f} is halogen.

In certain embodiments, R¹ is selected from methyl, ethyl, isopropyl, CF₃, —OCH₃, —OCH₂CH₃, —OCH(CH₃)₂, —OCH₂CH₂F, —OCH₂CH₂OH, —OCH₂CH₂OCH₃, —OCH₂CH₂N(CH₃)₂, —NHCH₃, —N(CH₃)₂, —NC(═O)CH₃, —SCH₃, cyclopropyl, 1-methyl-cyclopropyl and furany-2-yl.

In certain embodiments, R¹ is selected from methyl, ethyl, isopropyl, CF₃, OCH₃, —OCH₂CH₃, —OCH(CH₃)₂, —OCH₂CH₂F, —OCH₂CH₂OH, —OCH₂CH₂OCH₃, —OCH₂CH₂N(CH₃)₂, —NHCH₃, —N(CH₃)₂, —SCH₃, cyclopropyl, 1-methyl-cyclopropyl and furany-2-yl.

In certain embodiments, R¹ is C₁-C₆ alkyl optionally substituted with one to three R^{f} groups. In certain embodiments, each R^{f} is independently selected from halogen, OH, OCH₃, oxo, NR^{a}R^{b}, or C₃-C₆ cycloalkyl. In certain embodiments, each R^{f} is halogen. In certain embodiments, R¹ is selected from methyl, ethyl, isopropyl and CF₃.

In certain embodiments, R¹ is OR^{c}. In certain embodiments, R^{c} is selected from a 4-6 membered heterocyclyl and C₁-C₆ alkyl optionally substituted with halogen, OH, OCH₃, C₃-C₆ cycloalkyl, a 4-6 membered heterocyclyl or NR^{a}R^{b}. In certain embodiments, R^{c} is C₁-C₆ alkyl optionally substituted with halogen, OH, OCH₃ or NR^{a}R^{b}. In certain embodiments, R^{a} and R^{b} are independently selected from hydrogen and C₁-C₄ alkyl. In certain embodiments, R¹ is selected from —OCH₃, —OCH₂CH₃, —OCH(CH₃)₂, —OCH₂CH₂F, —OCH₂CH₂OH, —OCH₂CH₂OCH₃ and —OCH₂CH₂N(CH₃)₂.

In certain embodiments, R¹ is NR^{a}R^{b}. In certain embodiments, R^{a} and R^{b} are independently selected from hydrogen, phenyl and C₁-C₄ alkyl optionally substituted with oxo.

In certain embodiments, R^{a} and R^{b} are independently selected from hydrogen and C₁-C₄ alkyl optionally substituted with alkyl. In certain embodiments, R¹ is selected from —NHCH₃, —N(CH₃)₂ and —NC(═O)CH₃.

In certain embodiments, R¹ is NR^{a}R^{b}. In certain embodiments, R^{a} and R^{b} are independently selected from hydrogen, phenyl and C₁-C₄ alkyl optionally substituted with oxo.

In certain embodiments, R^{a} and R^{b} are independently selected from hydrogen and C₁-C₄ alkyl. In certain embodiments, R¹ is selected from —NHCH₃ and —N(CH₃)₂.

In certain embodiments, R¹ is SR^{d}. In certain embodiments, R^{d} is C₁-C₆ alkyl. In certain embodiments, R¹ is —SCH₃.

In certain embodiments, R¹ is C₃-C₆ cycloalkyl optionally substituted with halogen or C₁-C₄ alkyl. In certain embodiments, R¹ is C₃-C₆ cycloalkyl. In certain embodiments, R¹ is selected from cyclopropyl and 1-methyl-cyclopropyl.

In certain embodiments, R¹ is a 5-6 membered heteroaryl optionally substituted with C₁-C₄ alkyl. In certain embodiments, R¹ is a 5-6 membered heteroaryl. In certain embodiments, R¹ is a 5-6 membered heteroaryl, wherein the heteroaryl contains one, two or three heteroatoms selected from oxygen, nitrogen and sulfur. In certain embodiments, R¹ is a 5-6 membered heteroaryl, wherein the heteroaryl is furanyl. In certain embodiments, R¹ is furany-2-yl.

In certain embodiments, R², R³, R⁴ and R⁵ are independently selected from hydrogen, halogen and C₁-C₃ alkyl. In certain embodiments, R², R³, R⁴ and R⁵ are independently selected from hydrogen, halogen and methyl. In certain embodiments, R², R³, R⁴ and R⁵ are independently selected from hydrogen, F, Cl and methyl.

In certain embodiments, R² and R³ are independently selected from hydrogen, halogen, C₁-C₃ alkyl and C₁-C₃ alkoxy.

In certain embodiments, R² and R⁴ are independently selected from hydrogen, halogen or C₁-C₃ alkyl; R³ is Cl; and R⁵ is hydrogen or F. In certain embodiments, R² and R⁴ are independently selected from hydrogen, F, Cl and methyl; R³ is Cl; and R⁵ is hydrogen or F.

In certain embodiments, R² is hydrogen, halogen, C₁-C₃ alkyl or C₁-C₃ alkoxy.

In certain embodiments, R² is hydrogen.

In certain embodiments, R² is halogen. In certain embodiments, R² is F or Cl.

In certain embodiments, R² is C₁-C₃ alkyl. In certain embodiments, R² is methyl.

In certain embodiments, R³ is hydrogen, halogen, C₁-C₃ alkyl or C₁-C₃ alkoxy.

In certain embodiments, R³ is hydrogen.

In certain embodiments, R³ is halogen. In certain embodiments, R³ is F or Cl.

In certain embodiments, R³ is C₁-C₃ alkyl. In certain embodiments, R³ is methyl.

In certain embodiments, R³ is Cl.

In certain embodiments, R³ is hydrogen.

In certain embodiments, R⁴ is hydrogen, halogen or C₁-C₃ alkyl.

In certain embodiments, R⁴ is hydrogen.

In certain embodiments, R⁴ is halogen. In certain embodiments, R⁴ is F or Cl.

In certain embodiments, R⁵ is hydrogen, halogen or C₁-C₃ alkyl. In certain embodiments, R⁵ is hydrogen or halogen. In certain embodiments, R⁵ is hydrogen or F.

In certain embodiments, R² and R³ are F; and R⁴ and R⁵ are hydrogen.

In certain embodiments, R² is F; R³ is Cl; and R⁴ and R⁵ are hydrogen.

In certain embodiments, R² is Cl; R³ is F; and R⁴ and R⁵ are hydrogen.

In certain embodiments, R² is F, and R³, R⁴ and R⁵ are hydrogen.

In certain embodiments, R², R⁴ and R⁵ are hydrogen, and R³ is F.

In certain embodiments, R³ and R⁴ are F, and R² and R⁵ are hydrogen.

In certain embodiments, R² is Cl, and R³, R⁴ and R⁵ are hydrogen.

In certain embodiments, R², R³ and R⁴ are F, and R⁵ is hydrogen.

In certain embodiments, R² is F; R³ is methyl; and R⁴ and R⁵ are hydrogen.

In certain embodiments, R² is methyl; R³ is F; and R⁴ and R⁵ are hydrogen.

In certain embodiments, R² is F, and R³, R⁴ and R⁵ are hydrogen.

In certain embodiments, R² is Cl, and R³, R⁴ and R⁵ are hydrogen.

In certain embodiments, R³ is F, and R², R⁴ and R⁵ are hydrogen.

In certain embodiments, R², R³ and R⁵ are F, and R⁴ is hydrogen.

In certain embodiments, the residue:

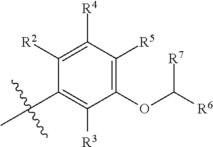

of Formula I, wherein the wavy line represents the point of attachment of the residue in Formula I, is selected from:

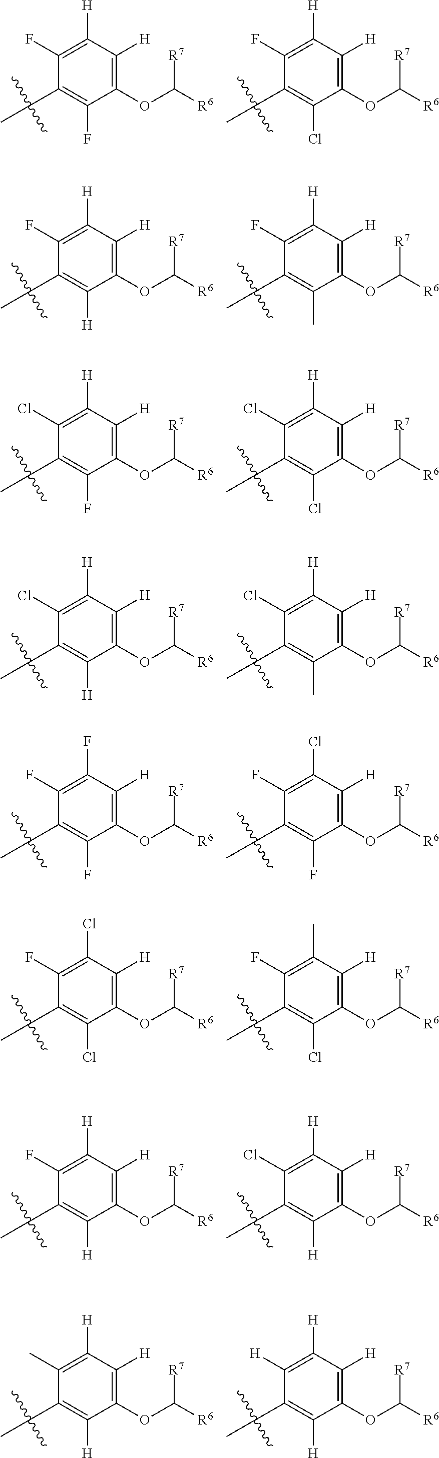

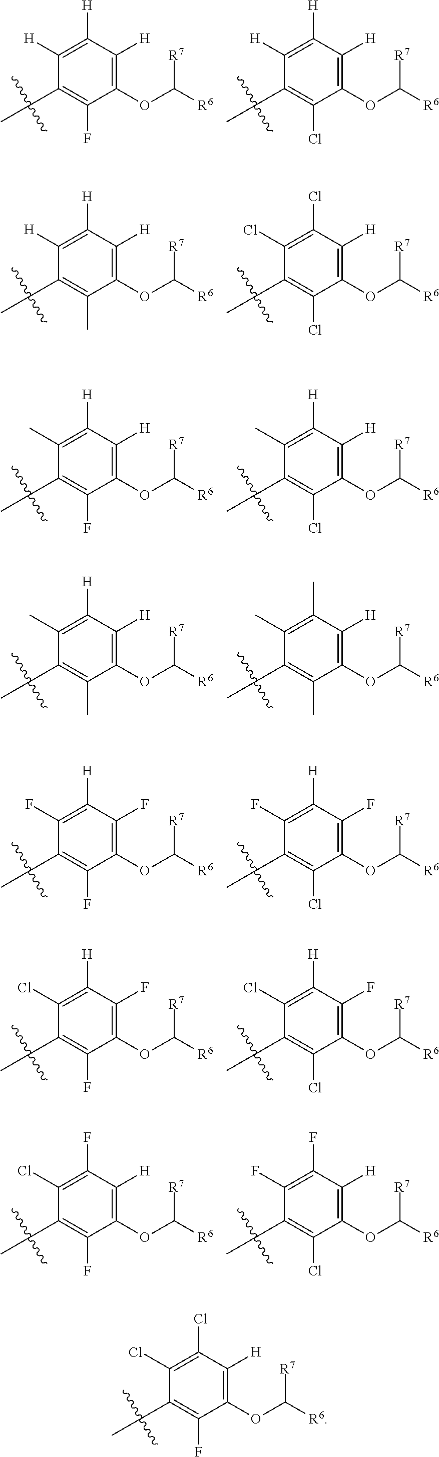

In certain embodiments, R⁶ is selected from phenyl, a 5-6 membered heteroaryl, a 9-10 membered bicyclic heterocyclyl or a 9-10 membered bicyclic heteroaryl, wherein the phenyl, heteroaryls and heterocyclyl are optionally substituted with one, two or three R^{g} groups. In certain embodiments, R^{g} is selected from halogen, CN, SO₂CH₃, C₁-C₃ alkyl, C₁-C₃ alkoxy, wherein the alkyl is optionally substituted with halogen or a 3-6 membered heterocyclyl. In certain embodiments, R⁶ is selected from phenyl, 2-fluorophenyl, 3-fluorophenyl, 4-fluorophenyl, 2-chlorophenyl, 3-chlorophenyl, 4-chlorophenyl, 4-bromophenyl, 4-iodophenyl, 2,4-difluorophenyl, 2-fluoro-4-chlorophenyl, 4-methylphenyl, 4-ethylphenyl, 4-trifluoromethylphenyl, 4-methoxyphenyl, 4-cyanophenyl, 4-(methylsulfonyl)phenyl, 3-(morpholinomethyl)phenyl, 4-(morpholinomethyl)phenyl, 4-cyclopropylphenyl, furan-2-yl, 1-methyl-1H-pyrazol-3-yl, 1-methyl-1H-pyrazol-4-yl, 5-methylisoxazol-3-yl, thiazol-2-yl, thiazol-4-yl, 2-methylthiazol-4-yl, 4-methylthiazol-5-yl, pyridin-2-yl, pyridin-3-yl, pyridin-4-yl, 5-chloropyridin-2-yl, 5-methylpyridin-2-yl, 6-methylpyridin-2-yl, 6-methylpyridin-3-yl, 5-(trifluoromethyl)pyridin-2-yl, 6-(trifluoromethyl)pyridin-3-yl, pyrimidin-2-yl, pyrimidin-4-yl, pyrimidin-5-yl, pyrazin-2-yl, 2,3-dihydrobenzofuran-5-yl, benzo[d][1,3]dioxol-5-yl, quinolin-6-yl and 1H-indol-4-yl.

In certain embodiments, R⁶ is selected from phenyl, a 5-6 membered heteroaryl, a 9-10 membered bicyclic heterocyclyl or a 9-10 membered bicyclic heteroaryl, wherein the phenyl, heteroaryls and heterocyclyl are optionally substituted with one, two or three R^{g} groups. In certain embodiments, R^{g} is selected from halogen, CN, SO₂CH₃, C₁-C₃ alkyl, C₁-C₃ alkoxy, wherein the alkyl is optionally substituted with halogen or a 3-6 membered heterocyclyl. In certain embodiments, R⁶ is selected from phenyl, 2-fluorophenyl, 3-fluorophenyl, 4-fluorophenyl, 2-chlorophenyl, 3-chlorophenyl, 4-chlorophenyl, 4-bromophenyl, 4-iodophenyl, 2,4-difluorophenyl, 2-fluoro-4-chlorophenyl, 4-methylphenyl, 4-ethylphenyl, 4-trifluoromethylphenyl, 4-methoxyphenyl, 4-cyanophenyl, 4-(methylsulfonyl)phenyl, 3-(morpholinomethyl)phenyl, furan-2-yl, 1-methyl-1H-pyrazol-3-yl, 1-methyl-1H-pyrazol-4-yl, 5-methylisoxazol-3-yl, thiazol-2-yl, thiazol-4-yl, 2-methylthiazol-4-yl, 4-methylthiazol-5-yl, pyridin-2-yl, pyridin-3-yl, pyridin-4-yl, 5-chloropyridin-2-yl, 5-methylpyridin-2-yl, 6-methylpyridin-2-yl, 6-methylpyridin-3-yl, 5-(trifluoromethyl)pyridin-2-yl, 6-(trifluoromethyl)pyridin-3-yl, pyrimidin-2-yl, pyrimidin-4-yl, pyrimidin-5-yl, pyrazin-2-yl, 2,3-dihydrobenzofuran-5-yl, benzo[d][1,3]dioxol-5-yl and quinolin-6-yl.

In certain embodiments, R^{g} is selected from halogen, CN, SO₂CH₃, C₁-C₃ alkyl, C₁-C₃ alkoxy, or C₃-C₆ cycloalkyl, wherein the alkyl is optionally substituted with halogen or a 3-6 membered heterocyclyl. In certain embodiments, R^{g} is selected from halogen, SO₂CH₃, C₁-C₃ alkyl, C₁-C₃ alkoxy, or C₃-C₆ cycloalkyl, wherein the alkyl is optionally substituted with halogen or a 3-6 membered heterocyclyl. In certain embodiments, R^{g} is selected from halogen and C₁-C₃ alkyl, wherein the alkyl is optionally substituted with halogen or a 3-6 membered heterocyclyl.

In certain embodiments, R^{g} is selected from halogen, CN, SO₂CH₃, C₁-C₃ alkyl, C₁-C₃ alkoxy, wherein the alkyl is optionally substituted with halogen or a 3-6 membered heterocyclyl. In certain embodiments, R^{g} is selected from halogen, SO₂CH₃, C₁-C₃ alkyl, C₁-C₃ alkoxy, wherein the alkyl is optionally substituted with halogen or a 3-6 membered heterocyclyl. In certain embodiments, R^{g} is selected from halogen and C₁-C₃ alkyl, wherein the alkyl is optionally substituted with halogen.

In certain embodiments, R⁶ is phenyl optionally substituted with one, two or three R^{g} groups. In certain embodiments, R⁶ is selected from phenyl, 2-fluorophenyl, 3-fluorophenyl, 4-fluorophenyl, 2-chlorophenyl, 3-chlorophenyl, 4-chlorophenyl, 4-bromophenyl, 4-iodophenyl, 2,4-difluorophenyl, 2-fluoro-4-chlorophenyl, 4-methylphenyl, 4-ethylphenyl, 4-trifluoromethylphenyl, 4-methoxyphenyl, 4-cyanophenyl, 4-(methylsulfonyl)phenyl, 3-(morpholinomethyl)phenyl, 4-(morpholinomethyl)phenyl and 4-cyclopropylphenyl.

In certain embodiments, R⁶ is phenyl optionally substituted with one, two or three R^{g} groups. In certain embodiments, R⁶ is selected from phenyl, 2-fluorophenyl, 3-fluorophenyl, 4-fluorophenyl, 2-chlorophenyl, 3-chlorophenyl, 4-chlorophenyl, 4-bromophenyl, 4-iodophenyl, 2,4-difluorophenyl, 2-fluoro-4-chlorophenyl, 4-methylphenyl, 4-ethylphenyl, 4-trifluoromethylphenyl, 4-methoxyphenyl, 4-cyanophenyl, 4-(methylsulfonyl)phenyl and 3-(morpholinomethyl)phenyl.

In certain embodiments, R⁶ is a 5-6 membered heteroaryl optionally substituted with one, two or three R^{g} groups. In certain embodiments, R⁶ is a 5-6 membered heteroaryl optionally substituted with one, two or three R^{g} groups, wherein the heteroaryl contains one, two or three heteroatoms selected from the group consisting of oxygen, nitrogen and sulfur. In certain embodiments, R⁶ is a 5-6 membered heteroaryl optionally substituted with one, two or three R^{g} groups, wherein the heteroaryl is selected from furanyl, pyrazolyl, isoxazolyl, thiazolyl, pyridinyl, pyrimidinyl and pyrazinyl. In certain embodiments, R⁶ is selected from furan-2-yl, 1-methyl-1H-pyrazol-3-yl, 1-methyl-1H-pyrazol-4-yl, 5-methylisoxazol-3-yl, thiazol-2-yl, thiazol-4-yl, 2-methylthiazol-4-yl, 4-methylthiazol-5-yl, pyridin-2-yl, pyridin-3-yl, pyridin-4-yl, 5-chloropyridin-2-yl, 5-methylpyridin-2-yl, 6-methylpyridin-2-yl, 6-methylpyridin-3-yl, 5-(trifluoromethyl)pyridin-2-yl, 6-(trifluoromethyl)pyridin-3-yl, pyrimidin-2-yl, pyrimidin-4-yl, pyrimidin-5-yl and pyrazin-2-yl.

In certain embodiments, R⁶ is a 9-10 membered bicyclic heterocyclyl optionally substituted with one, two or three R^{g} groups. In certain embodiments, R⁶ is a 9-10 membered bicyclic heterocyclyl. In certain embodiments, R⁶ is a 9-10 membered bicyclic heterocyclyl, wherein the heterocyclyl contains one, two or three heteroatoms selected from oxygen, nitrogen and sulfur. In certain embodiments, R⁶ is a 9-10 membered bicyclic heterocyclyl, wherein the heterocyclyl is selected from dihydrobenzofuranyl benzodioxolyl. In certain embodiments, R⁶ is selected from 2,3-dihydrobenzofuran-5-yl and benzo[d][1,3]dioxol-5-yl.

In certain embodiments, R⁶ is a 9-10 membered bicyclic heteroaryl optionally substituted with one, two or three R^{g} groups. In certain embodiments, R⁶ is a 9-10 membered bicyclic heteroaryl. In certain embodiments, R⁶ is a 9-10 membered bicyclic heteroaryl, wherein the heteroaryl contains one, two or three heteroatoms selected from oxygen, nitrogen and sulfur. In certain embodiments, R⁶ is a 9-10 membered bicyclic heteroaryl, wherein the heteroaryl is quinolinyl or indolyl. In certain embodiments, R⁶ is quinolin-6-yl or 1H-indol-4-yl.

In certain embodiments, R⁶ is a 9-10 membered bicyclic heteroaryl optionally substituted with one, two or three R^{g} groups. In certain embodiments, R⁶ is a 9-10 membered bicyclic heteroaryl. In certain embodiments, R⁶ is a 9-10 membered bicyclic heteroaryl, wherein the heteroaryl contains one, two or three heteroatoms selected from oxygen, nitrogen and sulfur. In certain embodiments, R⁶ is a 9-10 membered bicyclic heteroaryl, wherein the heteroaryl is quinolinyl. In certain embodiments, R⁶ is quinolin-6-yl.

In certain embodiments, R⁷ is selected from hydrogen and methyl.

In certain embodiments, R⁷ is hydrogen.

In certain embodiments, R⁷ is methyl.

It will be appreciated that certain compounds described herein may contain asymmetric or chiral centers, and therefore exist in different stereoisomeric forms. It is intended that all stereoisomeric forms of the compounds described herein, including but not limited to, diastereomers, enantiomers and atropisomers, as well as mixtures thereof such as racemic mixtures, form part of the present compounds.

In the structures shown herein, where the stereochemistry of any particular chiral atom is not specified, then all stereoisomers are contemplated and included as the compounds described herein. Where stereochemistry is specified by a solid wedge or dashed line representing a particular configuration, then that stereoisomer is so specified and defined.

It will also be appreciated that compounds of Formula I include tautomeric forms. Tautomers are compounds that are interconvertible by tautomerization. This commonly occurs due to the migration of a hydrogen atom or proton, accompanied by the switch of a single bond and adjacent double bond. For instance, 1H-pyrazolo[3,4-b]pyridine is one tautomeric form, while 7H-pyrazolo[3,4-b]pyridine is another tautomeric form. Other tautomers of Formula I may also form at other positions. The compounds of Formula I are intended to include all tautomeric forms.

The compounds of Formula I include the tautomer 7H-pyrazolo[3,4-b]pyridine, shown as Formula II:

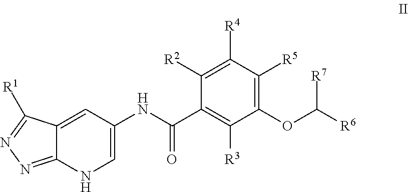

wherein R¹, R², R³, R⁴, R⁵, R⁶ and R⁷ are as defined herein.

One embodiment includes compounds of Formula I and named in Examples 1-78. One embodiment includes compounds of Formula I and named in Examples 1-21, 23-67, and 69-78. One embodiment includes compounds of Formula I and named in Examples 3-20, 23, 25-66, 69-74 and 76-78. One embodiment includes compounds of Formula I and named in Examples 4, 19, 29-31, 42-48, 51, 55, 56, 60, 62-66, 71-74 and 77.

It will also be appreciated that certain compounds of Formula I may be used as intermediates for further compounds of Formula I.

It will be further appreciated that the compounds described herein may exist in unsolvated as well as solvated forms with pharmaceutically acceptable solvents such as water, ethanol, and the like, and it is intended that the compounds embrace both solvated and unsolvated forms.

It will also be further appreciated that the compounds of Formula I include compounds that differ only in the presence of one or more isotopically enriched atoms. For example, compounds of Formulas I, wherein one or more hydrogen atoms are replaced deuterium or tritium, or one or more carbon atoms are replaced by a ¹³C- or ¹⁴C-enriched carbon are within the scope of this invention.

Synthesis of Compounds

Compounds described herein may be synthesized by synthetic routes that include processes analogous to those well-known in the chemical arts, particularly in light of the description contained herein. The starting materials are generally available from commercial sources such as Sigma-Aldrich (St. Louis, Mo.), Alfa Aesar (Ward Hill, Mass.), or TCI (Portland, Oreg.), or are readily prepared using methods well known to those skilled in the art (e.g., prepared by methods generally described in Louis F. Fieser and Mary Fieser, Reagents for Organic Synthesis. v. 1-23, New York: Wiley 1967-2006 ed. (also available via the Wiley InterScience® website), or Beilsteins Handbuch der organischen Chemie, 4, Aufl. ed. Springer-Verlag, Berlin, including supplements (also available via the Beilstein online database)).

For illustrative purposes, Schemes 1-10 show general methods for preparing the compounds described herein, as well as key intermediates. For a more detailed description of the individual reaction steps, see the Examples section below. Those skilled in the art will appreciate that other synthetic routes may be used to synthesize the compounds. Although specific starting materials and reagents are depicted in the Schemes and discussed below, other starting materials and reagents can be easily substituted to provide a variety of derivatives and/or reaction conditions. In addition, many of the compounds prepared by the methods described below can be further modified in light of this disclosure using conventional chemistry well known to those skilled in the art.

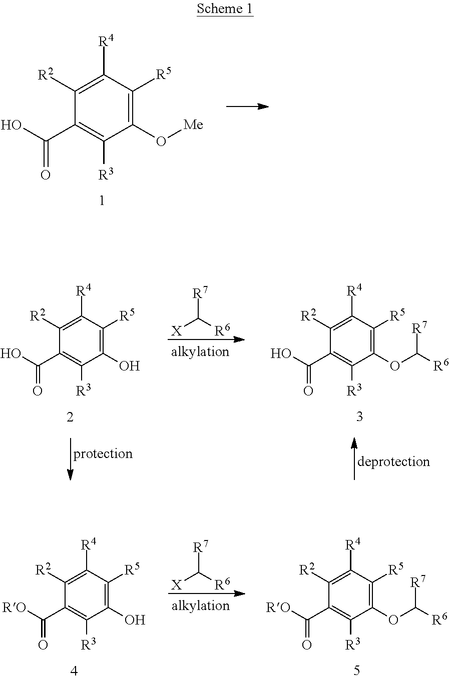

Scheme 1 shows a general method for preparing benzoic acid 3, wherein R², R³, R⁴, R⁵, R⁶ and R⁷ are as defined herein. Benzoic acid 1 is demethylated with a Lewis acid, such as BBr₃, to give hydroxyl benzoic acid 2. Compound 2 can be alkylated, wherein X is halogen, in the presence of a base, such as NaH or K₂CO₃, in a solvent, such as dimethylformamide (“DMF”) or tetrahydrofuran (“THF”), to give benzoic acid/ether 3. Alternatively, compound 2 can be protected via esterification, such as with TMSN₂ in MeOH to give ester 4, wherein R¹ is methyl. After alkylation to give ether 5, deprotection with an aqueous base, such as NaOH or KOH, in a solvent, such as MeOH, THF, or mixtures thereof, provides compound 3.

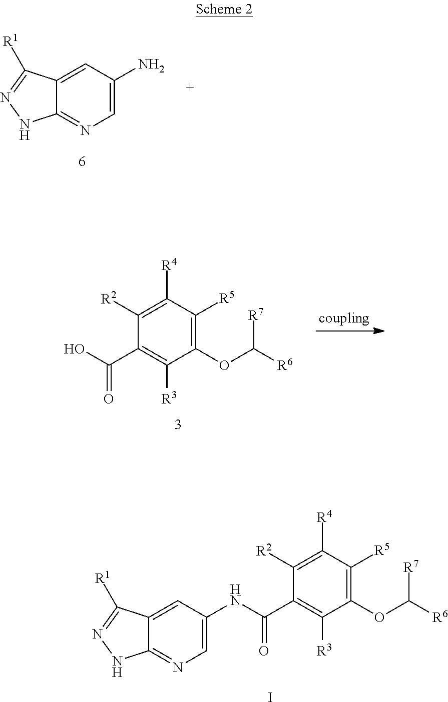

Scheme 2 shows a general method for preparing compounds of Formula I, wherein R¹, R², R³, R⁴, R⁵, R⁶ and R⁷ are as defined herein. Coupling of 5-aminopyrazolopyridine 6 with acid 3 is performed with an activating reagent, such as N-(3-dimethylaminopropyl)-N′-ethylcarbodiimide hydrochloride (“EDCI”), with or without the presence of an additive, such as hydroxybenzotriazole (“HOBt”), in a suitable solvent, such as DMF, THF or acetonitrile.

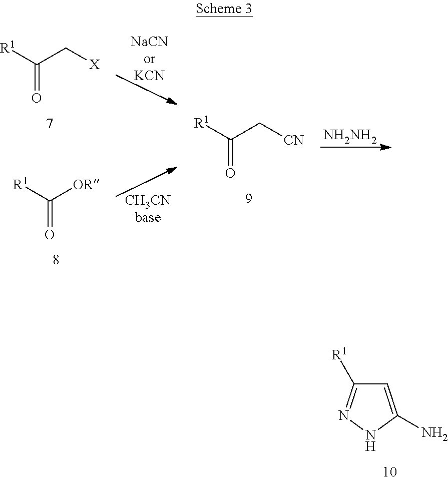

Scheme 3 shows a general method for preparing compound 10, wherein R¹ is as defined herein. α-Cyanoketone 9 may be prepared by reaction of an α-substituted ketone 7, wherein X is halogen or a suitable leaving group such as mesylate or tosylate, with NaCN or KCN in a suitable organic solvent, such as DMF. Alternatively, α-cyanoketone 9 is prepared by treatment of ester 8, wherein R″ is methyl, with CH₃CN and a suitable base, such as NaH or NaOtBu. Subjection of α-cyanoketone 9 to hydrazine in a solvent, such as EtOH, at a temperature of about 80° C. provides 3-substituted-1H-pyrazol-5-amine 10.

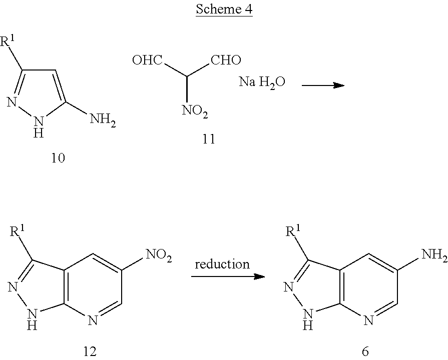

Scheme 4 shows a general method for preparing compound 6, wherein R¹ is as defined herein. Treatment of 3-substituted-1H-pyrazol-5-amine 10 with sodium nitromalonaldehyde monohydrate 11 in a suitable solvent, such as AcOH or water, at a temperature of about 90° C. affords 3-substituted-5-nitro-1H-pyrazolo[3,4-b]pyridine 12. Standard reduction of the nitro functionality in compound 12, such as by treatment with Pd/C and H₂, affords 3-substituted-1H-pyrazolo[3,4-b]pyridin-5-amine 6.

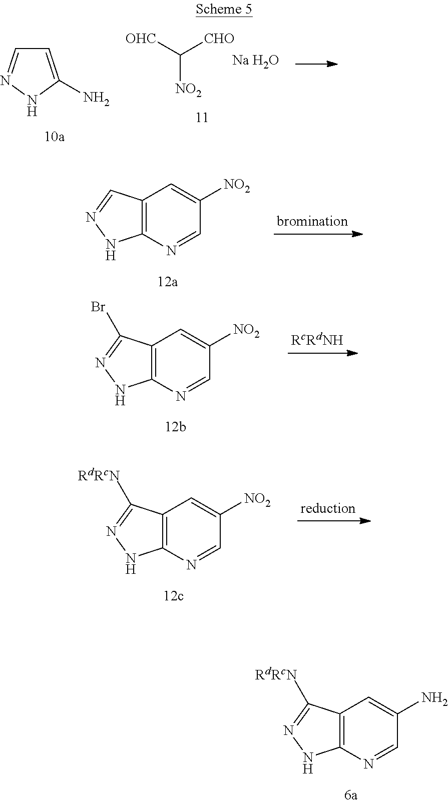

Scheme 5 shows a general method for preparing compounds 6a, wherein R^{c} and R^{d} are as defined herein. Treatment of 1H-pyrazol-5-amine 10a with sodium nitromalonaldehyde monohydrate 11 in a suitable solvent, such as AcOH or water, at 90° C. affords 5-nitro-1H-pyrazolo[3,4-b]pyridine 12a. Pyrazolopyridine 12a is brominated, for example, by bromine, in the presence of a base, such as NaOH, to give bromonitropyrazole 12b. Treatment of compound 12b with a nitrogen nucleophile at an elevated temperature, for example 120° C. to 160° C., affords 3-aminopyrazolo[3,4-b]pyridine 12c. Standard reduction of the nitro functionality in compound 12c, such as by treatment with Pd/C and H₂, affords 3-N-substituted-1H-pyrazolo[3,4-b]pyridin-5-amine 6a.

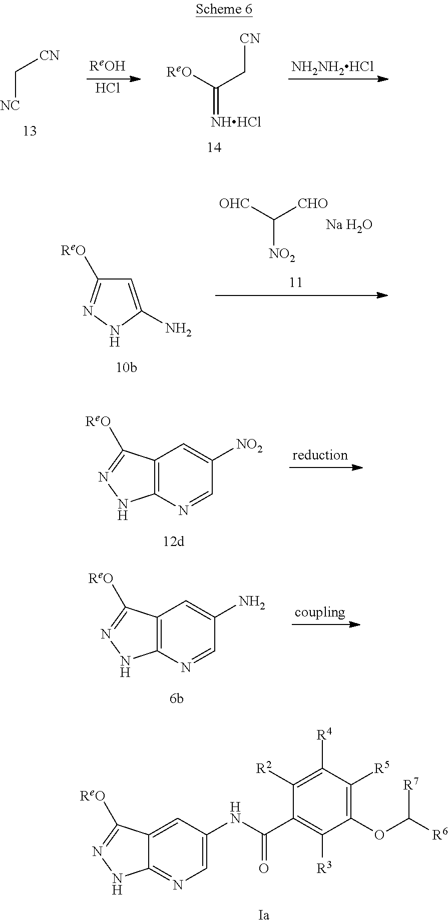

Scheme 6 shows a general method for preparing compounds of Formula Ia, wherein R², R³, R⁴, R⁵, R⁶, R⁷ and R^{e} are as defined herein. Malononitrile 13 is converted to imino ester HCl salt 14 by treatment with alcohol R^{e}OH in the presence of HCl in an organic solvent, such as diethyl ether. Compound 14 is then condensed with hydrazine monohydrochloride in a suitable solvent, such as MeOH, to provide 3-alkoxyl-1H-pyrazol-5-amine 10b. Cyclization of 10b with sodium nitromalonaldehyde monohydrate 11 in a suitable solvent, such as AcOH or H₂O, at 90° C. affords 3-alkoxyl-5-nitro-1H-pyrazolo[3,4-b]pyridine 12d. Standard reduction of the nitro functionality in compound 12d, such as by treatment with Pd/C and H₂, affords 3-substituted-1H-pyrazolo[3,4-b]pyridin-5-amine 6b. Standard amide-bond coupling provides pyrazolopyridine of Formula Ia.

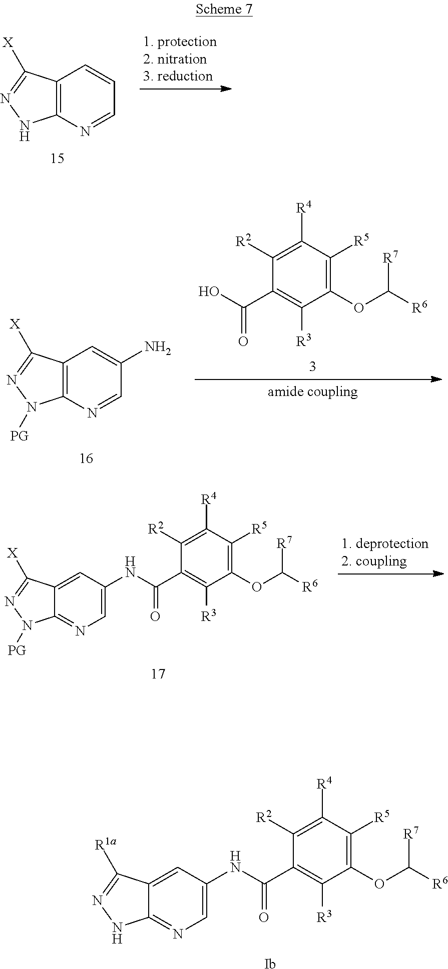

Scheme 7 illustrates a method for the installation of the R^{1a} group to provide compounds of Formula Ib, wherein R^{1a} is aryl or heteroaryl. Pyrazolopyridine 15, wherein X is halogen, may be protected, for example with a tosyl group by using tosyl chloride, in a solvent, such as dichloromethane or THF, in the presence of a base, such as K₂CO₃ or NaH. Nitration, for example with tetrabutylammonium nitrate, followed by reduction of the nitro group under standard conditions, such as SnCl₂ dihydrate, provides 5-amino pyrazolopyridine 16, wherein PG is a nitrogen protecting group, such as a tosyl group. Aniline 16 and benzoic acid 3 are coupled under standard conditions to provide amide 17. Removal of the protecting group under basic conditions, for example with K₂CO₃, at an appropriate temperature, for example 0° C. to reflux, followed by a cross-coupling reaction, for example the Suzuki, Stille or Negishi reactions, in the presence of a catalyst, such as tetrakis(triphenylphosphine)palladium, can be used to install a variety of aryl and heteroaryl groups providing pyrazolopyridine of Formula Ib.

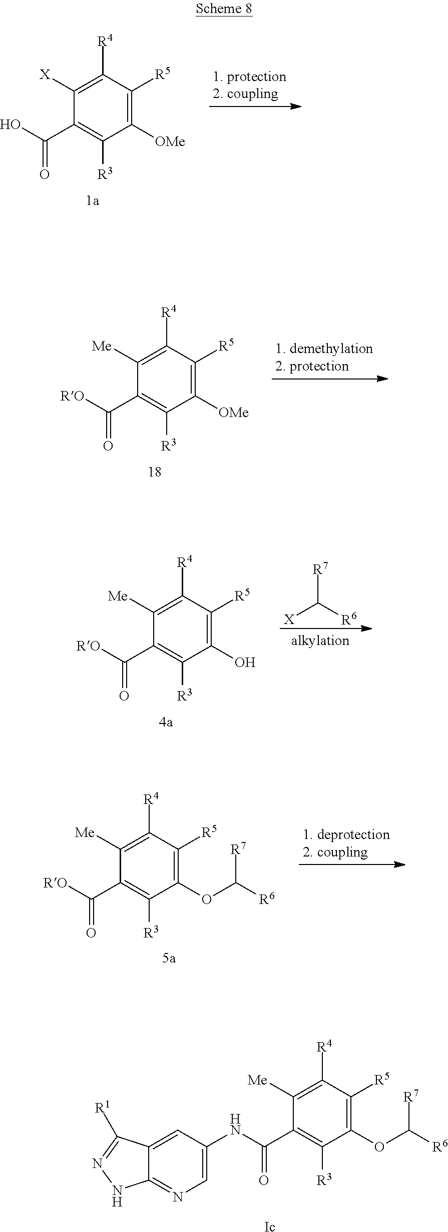

Scheme 8 illustrates a method for the installation of the Me group at the R² position to provide compounds of Formula Ic, wherein R¹, R³, R⁴, R⁵, R⁶, R⁷ and R′ are as defined herein. Benzoic acid 1a, wherein X is halogen, preferably Br or I, is protected via esterification, such as with TMSN₂, to give ester 18. Ester 18 is demethylated with a Lewis acid, such as BBr₃, and then reesterified to give phenolic ester 4a. Compound 4a can be alkylated, wherein X is halogen, in the presence of a base, such as NaH or K₂CO₃, in a solvent, such as DMF or THF, to give benzoic acid/ether 5a. Deprotection with an aqueous base, such as NaOH or KOH, in a solvent, such as MeOH, THF or mixtures thereof, followed by amide bond coupling provides pyrazolopyridine of Formula Ic.

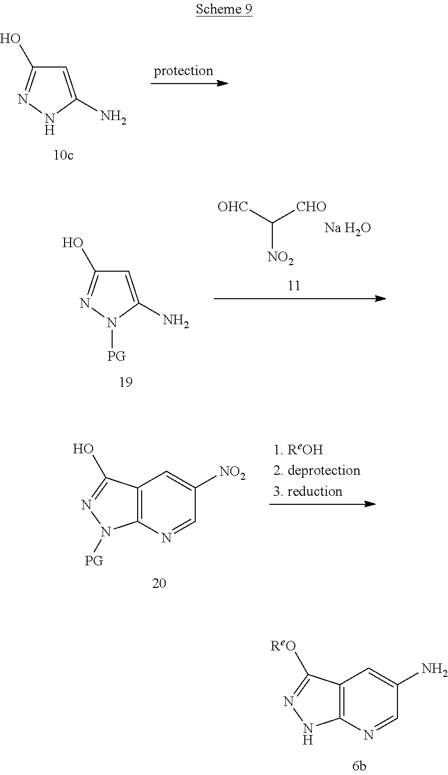

Scheme 9 illustrates an alternative method for the preparation of 3-substituted-1H-pyrazolo[3,4-b]pyridin-5-amines 6b, wherein R^{e} is as defined herein. Aminopyrazole 10c is protected with a protecting group giving compound 19, wherein PG is a protecting group such as toluenesulfonyl (“tosyl”). Cyclization of 19 with sodium nitromalonaldehyde monohydrate 11 in a suitable solvent, such as AcOH or H₂O, at 90° C. affords protected pyrazolopyridine 20. Compound 20 is converted to an ether with an alcohol via the Mitsunobu reaction utilizing, for example, triphenyl phosphine and diethyl azodicarboxylate. Deprotection under basic conditions, such as aqueous K₂CO₃ in THF or MeOH as solvent, followed by reduction under standard conditions provides 3-substituted-1H-pyrazolo[3,4-b]pyridin-5-amines 6b.

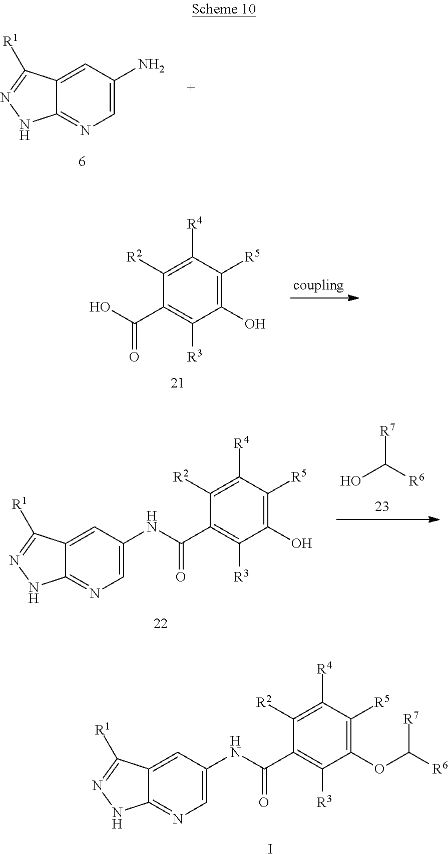

Scheme 10 illustrates an alternative method for preparing compounds of Formula I, wherein R¹, R², R³, R⁴, R⁵, R⁶ and R⁷ are as defined herein. Coupling of 5-aminopyrazolopyridine 6 with acid 21 is performed with an activating reagent, such as EDCI, in a suitable solvent, such as acetonitrile, to provide alcohol 22. Mitsunobu reaction of alcohol 22 with suitably substituted alcohol 23, in the presence of activating reagents, such as triphenyl phosphine and diethyl azodicarboxylate, provides pyrazolopyridine of Formula I.

In preparing compounds of Formula I, protection of remote functionalities (e.g., primary or secondary amines, etc.) of intermediates may be necessary. The need for such protection will vary depending on the nature of the remote functionality and the conditions of the preparation methods. Suitable amino-protecting groups (NH-Pg) include acetyl, trifluoroacetyl, t-butyloxycarbonyl (“Boc”), benzyloxycarbonyl (“CBz”) and 9-fluorenylmethyleneoxycarbonyl (“Fmoc”). The need for such protection is readily determined by one skilled in the art. For a general description of protecting groups and their use, see T. W. Greene, et al. Greene's Protective Groups in Organic Synthesis. New York: Wiley Interscience, 2006.

Accordingly, another embodiment provides a process for preparing compounds of Formulas I, Ia, Ib or Ic, comprising:

(a) coupling a compound of Formula 6:

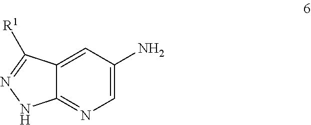

wherein R¹ is as defined herein;
with a compound of Formula 3:

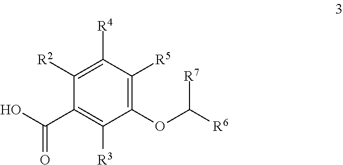

wherein R², R³, R⁴, R⁵, R⁶ and R⁷ are as defined herein;
to provide a compound of Formula I;

(b) coupling a compound of Formula 6b:

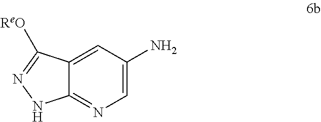

wherein R^{e} is as defined herein;
with a compound of Formula 3:

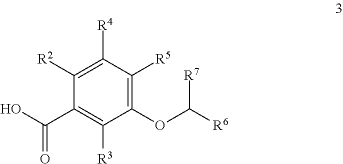

wherein R², R³, R⁴, R⁵, R⁶ and R⁷ are as defined herein;
to provide a compound of Formula Ia;

(c) deprotecting a compound of Formula 17:

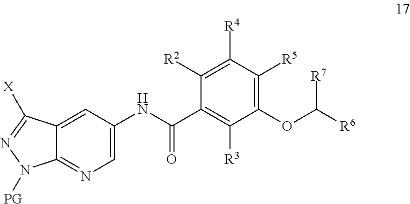

wherein R², R³, R⁴, R⁵, R⁶ and R⁷ are as defined herein, X is halogen, and PG is a nitrogen protecting group; and
performing a cross-coupling reaction in the presence of a catalyst to provide a compound of Formula Ib;

(d) deprotecting a compound of Formula 5a:

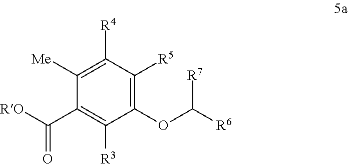

wherein R³, R⁴, R⁵, R⁶, R⁷ and R′ are as defined herein; and
coupling with a compound of Formula 6:

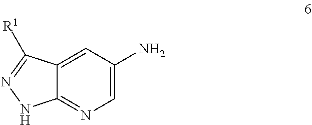

wherein R¹ is as defined herein;
to provide a compound of Formula Ic; and

(e) performing a Mitsunobu reaction with a compound of Formula 22:

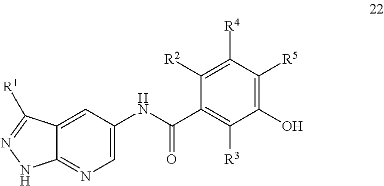

wherein R¹, R², R³, R⁴ and R⁵ are as defined herein;
and a compound of Formula 23:

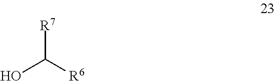

wherein R⁶ and R⁷ are as defined herein;
to provide a compound of Formula I.

Methods of Separation

It may be advantageous to separate reaction products from one another and/or from starting materials. The desired products of each step or series of steps is separated and/or purified (hereinafter separated) to the desired degree of homogeneity by the techniques common in the art. Typically such separations involve multiphase extraction, crystallization from a solvent or solvent mixture, distillation, sublimation, or chromatography. Chromatography can involve any number of methods including, for example: reverse-phase and normal phase; size exclusion; ion exchange; high, medium and low pressure liquid chromatography methods and apparatus; small scale analytical; simulated moving bed (“SMB”) and preparative thin or thick layer chromatography, as well as techniques of small scale thin layer and flash chromatography. One skilled in the art will apply techniques most likely to achieve the desired separation.

Diastereomeric mixtures can be separated into their individual diastereomers on the basis of their physical chemical differences by methods well known to those skilled in the art, such as by chromatography and/or fractional crystallization. Enantiomers can be separated by converting the enantiomeric mixture into a diastereomeric mixture by reaction with an appropriate optically active compound (e.g., chiral auxiliary such as a chiral alcohol or Mosher's acid chloride), separating the diastereomers and converting (e.g., hydrolyzing) the individual diastereoisomers to the corresponding pure enantiomers. Enantiomers can also be separated by use of a chiral HPLC column.

A single stereoisomer, e.g., an enantiomer, substantially free of its stereoisomer may be obtained by resolution of the racemic mixture using a method such as formation of diastereomers using optically active resolving agents (Eliel, E. and Wilen, S. Stereochemistry of Organic Compounds. New York: John Wiley & Sons, Inc., 1994; Lochmuller, C. H., et al. “Chromatographic resolution of enantiomers: Selective review.” J. Chromatogr., 113(3) (1975): pp. 283-302). Racemic mixtures of chiral compounds described herein may be separated and isolated by any suitable method, including: (1) formation of ionic, diastereomeric salts with chiral compounds and separation by fractional crystallization or other methods, (2) formation of diastereomeric compounds with chiral derivatizing reagents, separation of the diastereomers, and conversion to the pure stereoisomers, and (3) separation of the substantially pure or enriched stereoisomers directly under chiral conditions. See: Wainer, Irving W., Ed. Drug Stereochemistry: Analytical Methods and Pharmacology. New York: Marcel Dekker, Inc., 1993.

Under method (1), diastereomeric salts can be formed by reaction of enantiomerically pure chiral bases such as brucine, quinine, ephedrine, strychnine, α-methyl-β-phenylethylamine (amphetamine), and the like with asymmetric compounds bearing acidic functionality, such as carboxylic acid and sulfonic acid. The diastereomeric salts may be induced to separate by fractional crystallization or ionic chromatography. For separation of the optical isomers of amino compounds, addition of chiral carboxylic or sulfonic acids, such as camphorsulfonic acid, tartaric acid, mandelic acid, or lactic acid, can result in formation of the diastereomeric salts.

Alternatively, by method (2), the substrate to be resolved is reacted with one enantiomer of a chiral compound to form a diastereomeric pair (Eliel, E. and Wilen, S. Stereochemistry of Organic Compounds. New York: John Wiley & Sons, Inc., 1994, p. 322). Diastereomeric compounds can be formed by reacting asymmetric compounds with enantiomerically pure chiral derivatizing reagents, such as menthyl derivatives, followed by separation of the diastereomers and hydrolysis to yield the pure or enriched enantiomer. A method of determining optical purity involves making chiral esters, such as a menthyl ester, e.g., (−) menthyl chloroformate in the presence of base, or Mosher ester, α-methoxy-α-(trifluoromethyl)phenyl acetate (Jacob III, Peyton. “Resolution of (±)-5-Bromonornicotine. Synthesis of (R)- and (S)-Nornicotine of High Enantiomeric Purity.” J. Org. Chem. Vol. 47, No. 21 (1982): pp. 4165-4167), of the racemic mixture, and analyzing the ¹H NMR spectrum for the presence of the two atropisomeric enantiomers or diastereomers. Stable diastereomers of atropisomeric compounds can be separated and isolated by normal- and reverse-phase chromatography following methods for separation of atropisomeric naphthyl-isoquinolines (WO 96/15111).

By method (3), a racemic mixture of two enantiomers can be separated by chromatography using a chiral stationary phase (Lough, W. J., Ed. Chiral Liquid Chromatography. New York: Chapman and Hall, 1989; Okamoto, Yoshio, et al. “Optical resolution of dihydropyridine enantiomers by high-performance liquid chromatography using phenylcarbamates of polysaccharides as a chiral stationary phase.” J. of Chromatogr. Vol. 513 (1990): pp. 375-378). Enriched or purified enantiomers can be distinguished by methods used to distinguish other chiral molecules with asymmetric carbon atoms, such as optical rotation and circular dichroism.

Biological Evaluation

B-Raf mutant protein 447-717 (V600E) was co-expressed with the chaperone protein Cdc37, complexed with Hsp90 (Roe, S. Mark, et al. “The Mechanism of Hsp90 Regulation by the Protein Kinase-Specific Cochaperone p50^{cdc37}.” Cell. Vol. 116 (2004): pp. 87-98; Stancato, L F, et al. “Raf exists in a native heterocomplex with Hsp90 and p50 that can be reconstituted in a cell free system.” J. Biol. Chem. 268(29) (1993): pp. 21711-21716).

Determining the activity of Raf in the sample is possible by a number of direct and indirect detection methods (US 2004/0082014). Activity of human recombinant B-Raf protein may be assessed in vitro by assay of the incorporation of radio labeled phosphate to recombinant MAP kinase (MEK), a known physiologic substrate of B-Raf, according to US 2004/0127496 and WO 03/022840. The activity/inhibition of V600E full-length B-Raf was estimated by measuring the incorporation of radio labeled phosphate from [γ-³³]ATP into FSBA-modified wild-type MEK (see Biological Example 1).

Administration and Pharmaceutical Formulations

The compounds described herein may be administered by any convenient route appropriate to the condition to be treated. Suitable routes include oral, parenteral (including subcutaneous, intramuscular, intravenous, intraarterial, intradermal, intrathecal and epidural), transdermal, rectal, nasal, topical (including buccal and sublingual), vaginal, intraperitoneal, intrapulmonary and intranasal.

The compounds may be administered in any convenient administrative form, e.g., tablets, powders, capsules, solutions, dispersions, suspensions, syrups, sprays, suppositories, gels, emulsions, patches, etc. Such compositions may contain components conventional in pharmaceutical preparations, e.g., diluents, carriers, pH modifiers, sweeteners, bulking agents, and further active agents. If parenteral administration is desired, the compositions will be sterile and in a solution or suspension form suitable for injection or infusion.

A typical formulation is prepared by mixing a compound described herein and a carrier or excipient. Suitable carriers and excipients are well known to those skilled in the art and are described in detail in, e.g., Ansel, Howard C., et al., Ansel's Pharmaceutical Dosage Forms and Drug Delivery Systems. Philadelphia: Lippincott, Williams & Wilkins, 2004; Gennaro, Alfonso R., et al. Remington: The Science and Practice of Pharmacy. Philadelphia: Lippincott, Williams & Wilkins, 2000; and Rowe, Raymond C. Handbook of Pharmaceutical Excipients. Chicago, Pharmaceutical Press, 2005. The formulations may also include one or more buffers, stabilizing agents, surfactants, wetting agents, lubricating agents, emulsifiers, suspending agents, preservatives, antioxidants, opaquing agents, glidants, processing aids, colorants, sweeteners, perfuming agents, flavoring agents, diluents and other known additives to provide an elegant presentation of the drug (i.e., a compound described herein or pharmaceutical composition thereof) or aid in the manufacturing of the pharmaceutical product (i.e., medicament).

One embodiment includes a pharmaceutical composition comprising a compound of Formula I, or a stereoisomer, tautomer or pharmaceutically acceptable salt thereof. A further embodiment provides a pharmaceutical composition comprising a compound of Formula I, or a stereoisomer, tautomer or pharmaceutically acceptable salt thereof, together with a pharmaceutically acceptable carrier or excipient.

Methods of Treatment with Compounds of the Invention

Also provided are methods of treating or preventing disease or condition by administering one or more compounds described herein, or a stereoisomer or pharmaceutically acceptable salt thereof. In one embodiment, a human patient is treated with a compound of Formula I, or a stereoisomer, tautomer or pharmaceutically acceptable salt thereof, and a pharmaceutically acceptable carrier, adjuvant, or vehicle in an amount to detectably inhibit B-Raf activity.

In another embodiment, a method of treating a hyperproliferative disease in a mammal comprising administering a therapeutically effective amount of the compound of Formula I, or a stereoisomer, tautomer or pharmaceutically acceptable salt thereof, to the mammal is provided.

In another embodiment, a method of treating cancer in a mammal comprising administering a therapeutically effective amount of the compound of Formula I, or a stereoisomer, tautomer or pharmaceutically acceptable salt thereof, to the mammal is provided.

In another embodiment, a method of treating a kidney disease in a mammal comprising administering a therapeutically effective amount of the compound of Formula I, or a stereoisomer, tautomer or pharmaceutically acceptable salt thereof, to the mammal is provided. In a further embodiment, the kidney disease is polycystic kidney disease.

In another embodiment, a method of treating or preventing cancer in a mammal in need of such treatment, wherein the method comprises administering to said mammal a therapeutically effective amount of a compound of Formula I, or a stereoisomer, tautomer or pharmaceutically acceptable salt thereof. The cancer is selected from breast, ovary, cervix, prostate, testis, genitourinary tract, esophagus, larynx, glioblastoma, neuroblastoma, stomach, skin, keratoacanthoma, lung, epidermoid carcinoma, large cell carcinoma, NSCLC, small cell carcinoma, lung adenocarcinoma, bone, colon, adenoma, pancreas, adenocarcinoma, thyroid, follicular carcinoma, undifferentiated carcinoma, papillary carcinoma, seminoma, melanoma, sarcoma, bladder carcinoma, liver carcinoma and biliary passages, kidney carcinoma, myeloid disorders, lymphoid disorders, hairy cells, buccal cavity and pharynx (oral), lip, tongue, mouth, pharynx, small intestine, colon-rectum, large intestine, rectum, brain and central nervous system, Hodgkin's and leukemia. Another embodiment provides the use of a compound of Formula I, or a stereoisomer, tautomer or pharmaceutically acceptable salt thereof, in the manufacture of a medicament for the treatment of cancer.

In another embodiment, a method of treating or preventing kidney disease in a mammal in need of such treatment, wherein the method comprises administering to said mammal a therapeutically effective amount of a compound of Formula I, or a stereoisomer, tautomer or pharmaceutically acceptable salt thereof. In a further embodiment, the kidney disease is polycystic kidney disease.

In another embodiment, a method of treating or preventing a disease or disorder modulated by B-Raf, comprising administering to a mammal in need of such treatment an effective amount of a compound of Formula I, or a stereoisomer, tautomer or pharmaceutically acceptable salt thereof. Examples of such diseases and disorders include, but are not limited to, hyperproliferative diseases (including cancer) and kidney disease (including polycytic kidney disease).

Another embodiment provides the use of a compound of Formula I, or a stereoisomer, tautomer or pharmaceutically acceptable salt thereof, in the manufacture of a medicament for the treatment of a hyperproliferative disease.

Another embodiment provides the use of a compound of Formula I, or a stereoisomer, tautomer or pharmaceutically acceptable salt thereof, in the manufacture of a medicament for the treatment of cancer.

Another embodiment provides the use of a compound of Formula I, or a stereoisomer, tautomer or pharmaceutically acceptable salt thereof, in the manufacture of a medicament for the treatment of kidney disease. In a further embodiment, the kidney disease is polycystic kidney disease.

In another embodiment, a method of preventing or treating cancer, comprising administering to a mammal in need of such treatment an effective amount of a compound of Formula I, or a stereoisomer, tautomer or pharmaceutically acceptable salt thereof, alone or in combination with one or more additional compounds having anti-cancer properties.

Another embodiment of the present invention provides the compounds of Formula I for use in therapy.

Another embodiment of the present invention provides the compounds of Formula I for use in the treatment of a hyperproliferative disease. In a further embodiment, the hyperproliferative disease is cancer.

Another embodiment of the present invention provides the compounds of Formula I for use in the treatment of kidney disease. In a further embodiment, the kidney disease is polycystic kidney disease.

In one further embodiment, the cancer is selected from breast, ovary, cervix, prostate, testis, genitourinary tract, esophagus, larynx, glioblastoma, neuroblastoma, stomach, skin, keratoacanthoma, lung, epidermoid carcinoma, large cell carcinoma, NSCLC, small cell carcinoma, lung adenocarcinoma, bone, colon, adenoma, pancreas, adenocarcinoma, thyroid, follicular carcinoma, undifferentiated carcinoma, papillary carcinoma, seminoma, melanoma, sarcoma, bladder carcinoma, liver carcinoma and biliary passages, kidney carcinoma, myeloid disorders, lymphoid disorders, hairy cells, buccal cavity and pharynx (oral), lip, tongue, mouth, pharynx, small intestine, colon-rectum, large intestine, rectum, brain and central nervous system, Hodgkin's and leukemia.

In one further embodiment, the cancer is a sarcoma.

In another further embodiment, the cancer is a carcinoma. In one further embodiment, the carcinoma is squamous cell carcinoma. In another further embodiment, the carcinoma is an adenoma or adenocarcinoma.

Combination Therapy

The compounds described herein and stereoisomers and pharmaceutically acceptable salts thereof may be employed alone or in combination with other therapeutic agents for treatment. The compounds described herein may be used in combination with one or more additional drugs, for example an anti-hyperproliferative (or anti-cancer) agent that works through action on a different target protein. The second compound of the pharmaceutical combination formulation or dosing regimen preferably has complementary activities to the compound described herein, such that they do not adversely affect each other. Such molecules are suitably present in combination in amounts that are effective for the purpose intended. The compounds may be administered together in a unitary pharmaceutical composition or separately and, when administered separately this may occur simultaneously or sequentially in any order. Such sequential administration may be close in time or remote in time.

A “chemotherapeutic agent” is a chemical compound useful in the treatment of cancer, regardless of mechanism of action. Chemotherapeutic agents include compounds used in “targeted therapy” and conventional chemotherapy. A number of suitable chemotherapeutic agents to be used as combination therapeutics are contemplated for use in the methods of the present invention. The present invention contemplates, but is not limited to, administration of numerous anticancer agents, such as: agents that induce apoptosis; polynucleotides (e.g., ribozymes); polypeptides (e.g., enzymes); drugs; biological mimetics; alkaloids; alkylating agents; antitumor antibiotics; antimetabolites; hormones; platinum compounds; monoclonal antibodies conjugated with anticancer drugs, toxins, and/or radionuclides; biological response modifiers (e.g., interferons [e.g., IFN-a, etc.] and interleukins [e.g., IL-2, etc.], etc.); adoptive immunotherapy agents; hematopoietic growth factors; agents that induce tumor cell differentiation (e.g., all-trans-retinoic acid, etc.); gene therapy reagents; antisense therapy reagents and nucleotides; tumor vaccines; inhibitors of angiogenesis, and the like.

Examples of chemotherapeutic agents include Erlotinib (TARCEVA®, Genentech/OSI Pharm.), Bortezomib (VELCADE®, Millennium Pharm.), Fulvestrant (FASLODEX®, Astra Zeneca), Sunitinib (SUTENT®, Pfizer), Letrozole (FEMARA®, Novartis), Imatinib mesylate (GLEEVEC®, Novartis), PTK787/ZK 222584 (Novartis), Oxaliplatin (Eloxatin®, Sanofi), 5-FU (5-fluorouracil), Leucovorin, Rapamycin (Sirolimus, RAPAMUNE®, Wyeth), Lapatinib (TYKERB®, GSK572016, Glaxo Smith Kline), Lonafarnib (SCH 66336), Sorafenib (NEXAVAR®, Bayer), Irinotecan (CAMPTOSAR®, Pfizer) and Gefitinib (IRESSA®, AstraZeneca), AG1478, AG1571 (SU 5271; Sugen), alkylating agents such as thiotepa and CYTOXAN® cyclosphosphamide; alkyl sulfonates such as busulfan, improsulfan and piposulfan; aziridines such as benzodopa, carboquone, meturedopa, and uredopa; ethylenimines and methylamelamines including altretamine, triethylenemelamine, triethylenephosphoramide, triethylenethiophosphoramide and trimethylomelamine; acetogenins (especially bullatacin and bullatacinone); a camptothecin (including the synthetic analog topotecan); bryostatin; callystatin; CC-1065 (including its adozelesin, carzelesin and bizelesin synthetic analogs); cryptophycins (particularly cryptophycin 1 and cryptophycin 8); dolastatin; duocarmycin (including the synthetic analogs, KW-2189 and CB1-TM1); eleutherobin; pancratistatin; a sarcodictyin; spongistatin; nitrogen mustards such as chlorambucil, chlornaphazine, chlorophosphamide, estramustine, ifosfamide, mechlorethamine, mechlorethamine oxide hydrochloride, melphalan, novembichin, phenesterine, prednimustine, trofosfamide, uracil mustard; nitrosureas such as carmustine, chlorozotocin, fotemustine, lomustine, nimustine, and ranimnustine; antibiotics such as the enediyne antibiotics (e.g., calicheamicin, especially calicheamicin gamma1I and calicheamicin omegaI1(Angew Chem. Intl. Ed. Engl. (1994) 33:183-186); dynemicin, including dynemicin A; bisphosphonates, such as clodronate; an esperamicin; as well as neocarzinostatin chromophore and related chromoprotein enediyne antibiotic chromophores), aclacinomysins, actinomycin, authramycin, azaserine, bleomycins, cactinomycin, carabicin, caminomycin, carzinophilin, chromomycinis, dactinomycin, daunorubicin, detorubicin, 6-diazo-5-oxo-L-norleucine, ADRIAMYCIN® (doxorubicin), morpholino-doxorubicin, cyanomorpholino-doxorubicin, 2-pyrrolino-doxorubicin and deoxydoxorubicin), epirubicin, esorubicin, idarubicin, marcellomycin, mitomycins such as mitomycin C, mycophenolic acid, nogalamycin, olivomycins, peplomycin, porfiromycin, puromycin, quelamycin, rodorubicin, streptonigrin, streptozocin, tubercidin, ubenimex, zinostatin, zorubicin; anti-metabolites such as methotrexate and 5-fluorouracil (5-FU); folic acid analogs such as denopterin, methotrexate, pteropterin, trimetrexate; purine analogs such as fludarabine, 6-mercaptopurine, thiamiprine, thioguanine; pyrimidine analogs such as ancitabine, azacitidine, 6-azauridine, carmofur, cytarabine, dideoxyuridine, doxifluridine, enocitabine, floxuridine; androgens such as calusterone, dromostanolone propionate, epitiostanol, mepitiostane, testolactone; anti-adrenals such as aminoglutethimide, mitotane, trilostane; folic acid replenisher such as frolinic acid; aceglatone; aldophosphamide glycoside; aminolevulinic acid; eniluracil; amsacrine; bestrabucil; bisantrene; edatraxate; defofamine; demecolcine; diaziquone; elfornithine; elliptinium acetate; an epothilone; etoglucid; gallium nitrate; hydroxyurea; lentinan; lonidainine; maytansinoids such as maytansine and ansamitocins; mitoguazone; mitoxantrone; mopidanmol; nitraerine; pentostatin; phenamet; pirarubicin; losoxantrone; podophyllinic acid; 2-ethylhydrazide; procarbazine; PSK® polysaccharide complex (JHS Natural Products, Eugene, Oreg.); razoxane; rhizoxin; sizofuran; spirogermanium; tenuazonic acid; triaziquone; 2,2′,2″-trichlorotriethylamine; trichothecenes (especially T-2 toxin, verracurin A, roridin A and anguidine); urethan; vindesine; dacarbazine; mannomustine; mitobronitol; mitolactol; pipobroman; gacytosine; arabinoside (“Ara-C”); cyclophosphamide; thiotepa; taxoids, e.g., TAXOL® (paclitaxel; Bristol-Myers Squibb Oncology, Princeton, N.J.), ABRAXANE™ (Cremophor-free), albumin-engineered nanoparticle formulations of paclitaxel (American Pharmaceutical Partners, Schaumberg, Ill.), and TAXOTERE® (doxetaxel; Rhone-Poulenc Rorer, Antony, France); chloranmbucil; GEMZAR® (gemcitabine); 6-thioguanine; mercaptopurine; methotrexate; platinum analogs such as cisplatin and carboplatin; vinblastine; etoposide (VP-16); ifosfamide; mitoxantrone; vincristine; NAVELBINE® (vinorelbine); novantrone; teniposide; edatrexate; daunomycin; aminopterin; capecitabine (XELODA®); ibandronate; CPT-11; topoisomerase inhibitor RFS 2000; difluoromethylornithine (DMFO); retinoids such as retinoic acid; and pharmaceutically acceptable salts, acids and derivatives of any of the above.

Also included in the definition of “chemotherapeutic agent” are: (i) anti-hormonal agents that act to regulate or inhibit hormone action on tumors such as anti-estrogens and selective estrogen receptor modulators (SERMs), including, for example, tamoxifen (including NOLVADEX®; tamoxifen citrate), raloxifene, droloxifene, 4-hydroxytamoxifen, trioxifene, keoxifene, LY117018, onapristone, and FARESTON® (toremifene citrate); (ii) aromatase inhibitors that inhibit the enzyme aromatase, which regulates estrogen production in the adrenal glands, such as, for example, 4(5)-imidazoles, aminoglutethimide, MEGASE® (megestrol acetate), AROMASIN® (exemestane; Pfizer), formestanie, fadrozole, RIVISOR® (vorozole), FEMARA® (letrozole; Novartis), and ARIMIDEX® (anastrozole; AstraZeneca); (iii) anti-androgens such as flutamide, nilutamide, bicalutamide, leuprolide, and goserelin; as well as troxacitabine (a 1,3-dioxolane nucleoside cytosine analog); (iv) protein kinase inhibitors; (v) lipid kinase inhibitors; (vi) antisense oligonucleotides, particularly those which inhibit expression of genes in signaling pathways implicated in aberrant cell proliferation, such as, for example, PKC-alpha, Ralf and H-Ras; (vii) ribozymes such as VEGF expression inhibitors (e.g., ANGIOZYME®) and HER2 expression inhibitors; (viii) vaccines such as gene therapy vaccines, for example, ALLOVECTIN®, LEUVECTIN®, and VAXID®; PROLEUKIN® rIL-2; a topoisomerase 1 inhibitor such as LURTOTECAN®; ABARELIX® rmRH; (ix) anti-angiogenic agents such as bevacizumab (AVASTIN®, Genentech); (x) PI3k/AKT/mTOR pathway inhibitors, including GDC-0941 (2-(1H-Indazol-4-yl)-6-(4-methanesulfonyl-piperazin-1-ylmethyl)-4-morpholin-4-yl-thieno[3,2-d]pyrimidine), XL-147, GSK690693 and temsirolimus; (xi) Ras/MEK/ERK pathway inhibitors; and (xii) pharmaceutically acceptable salts, acids and derivatives of any of the above.

Also included in the definition of “chemotherapeutic agent” are therapeutic antibodies such as alemtuzumab (Campath), bevacizumab (AVASTIN®, Genentech); cetuximab (ERBITUX®, Imclone); panitumumab (VECTIBIX®, Amgen), rituximab (RITUXAN®, Genentech/Biogen Idec), pertuzumab (OMNITARG®, 2C4, Genentech), trastuzumab (HERCEPTIN®, Genentech), tositumomab (Bexxar, Corixia), and the antibody drug conjugate, gemtuzumab ozogamicin (MYLOTARG®, Wyeth).

Humanized monoclonal antibodies with therapeutic potential as chemotherapeutic agents in combination with the Raf inhibitors of the invention include: alemtuzumab, apolizumab, aselizumab, atlizumab, bapineuzumab, bevacizumab, bivatuzumab mertansine, cantuzumab mertansine, cedelizumab, certolizumab pegol, cidfusituzumab, cidtuzumab, daclizumab, eculizumab, efalizumab, epratuzumab, erlizumab, felvizumab, fontolizumab, gemtuzumab ozogamicin, inotuzumab ozogamicin, ipilimumab, labetuzumab, lintuzumab, matuzumab, mepolizumab, motavizumab, motovizumab, natalizumab, nimotuzumab, nolovizumab, numavizumab, ocrelizumab, omalizumab, palivizumab, pascolizumab, pecfusituzumab, pectuzumab, pertuzumab, pexelizumab, ralivizumab, ranibizumab, reslivizumab, reslizumab, resyvizumab, rovelizumab, ruplizumab, sibrotuzumab, siplizumab, sontuzumab, tacatuzumab tetraxetan, tadocizumab, talizumab, tefibazumab, tocilizumab, toralizumab, trastuzumab, tucotuzumab celmoleukin, tucusituzumab, umavizumab, urtoxazumab, and visilizumab.

### EXAMPLES

For illustrative purposes, the following Examples are included. However, it is to be understood that these Examples do not limit the invention and are only meant to suggest a method of practicing the invention. Persons skilled in the art will recognize that the chemical reactions described may be readily adapted to prepare a number of other compounds described herein, and alternative methods for preparing the compounds are deemed to be within the scope of this invention. For example, the synthesis of non-exemplified compounds may be successfully performed by modifications apparent to those skilled in the art, e.g., by appropriately protecting interfering groups, by utilizing other suitable reagents known in the art other than those described, and/or by making routine modifications of reaction conditions. Alternatively, other reactions disclosed herein or known in the art will be recognized as having applicability for preparing other compounds described herein.

In the Examples described below, unless otherwise indicated all temperatures are set forth in degrees Celsius. Reagents were purchased from commercial suppliers such as Sigma-Aldrich, Alfa Aesar, or TCI, and were used without further purification unless otherwise indicated.

The reactions set forth below were done generally under a positive pressure of nitrogen or argon or with a drying tube (unless otherwise stated) in anhydrous solvents, and the reaction flasks were typically fitted with rubber septa for the introduction of substrates and reagents via syringe. Glassware was oven dried and/or heat dried.

Column chromatography was done on a Biotage system (Manufacturer: Dyax Corporation) having a silica gel column or on a silica SepPak cartridge (Waters) (unless otherwise stated). ¹H NMR spectra were recorded on a Varian instrument operating at 400 MHz. ¹H-NMR spectra were obtained as CDCl₃, CD₃OD, D₂O, (CD₃)₂SO, (CD₃)₂CO, C₆D₆, CD₃CN solutions (reported in ppm), using tetramethylsilane (0.00 ppm) or residual solvent (CDCl₃: 7.26 ppm; CD₃OD: 3.31 ppm; D₂O: 4.79 ppm; (CD₃)₂SO: 2.50 ppm; (CD₃)₂CO: 2.05 ppm; C₆D₆: 7.16 ppm; CD₃CN: 1.94 ppm) as the reference standard. When peak multiplicities are reported, the following abbreviations are used: s (singlet), d (doublet), t (triplet), q (quartet), m (multiplet), br (broadened), dd (doublet of doublets), dt (doublet of triplets). Coupling constants, when given, are reported in Hertz (Hz).

### Biological Example 1

### B-Raf IC₅₀ Assay Protocol

Activity of human recombinant B-Raf protein may be assessed in vitro by assay of the incorporation of radio labeled phosphate to recombinant MAP kinase (MEK), a known physiologic substrate of B-Raf, according to US 2004/0127496 and WO 03/022840. Catalytically active human recombinant B-Raf protein is obtained by purification from sf9 insect cells infected with a human B-Raf recombinant baculovirus expression vector.

The activity/inhibition of V600E full-length B-Raf was estimated by measuring the incorporation of radio labeled phosphate from [γ-³³]ATP into FSBA-modified wild-type MEK. The 30-μL, assay mixtures contained 25 mM Na Pipes, pH 7.2, 100 mM KCl, 10 mM MgCl₂, 5 mM β-glycerophosphate, 100 μM Na Vanadate, 4 μM ATP, 500 nCi [γ-³³P]ATP, 1 μM FSBA-MEK and 20 nM V600E full-length B-Raf. Incubations were carried out at 22° C. in a Costar 3365 plate (Corning). Prior to the assay, the B-Raf and FSBA-MEK were preincubated together in assay buffer at 1.5× (20 μL of 30 nM and 1.5 μM, respectively) for 15 minutes, and the assay was initiated by the addition of 10 μL of 10 μM ATP. Following the 60-minute incubation, the assay mixtures were quenched by the addition of 100 μL of 25% TCA, the plate was mixed on a rotary shaker for 1 minute, and the product was captured on a Perkin-Elmer GF/B filter plate using a Tomtec Mach III Harvester. After sealing the bottom of the plate, 35 μL of Bio-Safe II (Research Products International) scintillation cocktail were added to each well and the plate was top-sealed and counted in a Topcount NXT (Packard).

The compounds of Examples 1-74 were tested in the above assay and found to have an IC₅₀ of less than 1 μM.

The compounds of Examples 1, 2, 3, 4, 5, 6, 7, 8, 9, 10, 11, 12, 13, 14, 15, 16, 17, 18, 19, 20, 21, 23, 24, 25, 26, 27, 28, 29, 30, 31, 32, 33, 34, 35, 36, 37, 38, 39, 40, 41, 42, 43, 44, 45, 46, 47, 48, 49, 50, 51, 52, 53, 54, 55, 56, 57, 58, 59, 60, 61, 62, 63, 64, 65, 66, 67, 69, 70, 71, 72, 73 and 74 were tested in the above assay and found to have an IC₅₀ of less than 500 nM.

The compounds of Examples 1, 2, 3, 4, 5, 6, 7, 8, 9, 10, 11, 12, 13, 14, 15, 16, 17, 18, 19, 20, 21, 23, 24, 25, 26, 27, 28, 29, 30, 31, 32, 33, 34, 35, 36, 37, 38, 39, 40, 41, 42, 43, 44, 45, 46, 47, 48, 49, 50, 51, 52, 53, 54, 55, 56, 57, 58, 59, 60, 61, 62, 63, 64, 65, 66, 67, 69, 70, 71, 72, 73, 74, 75, 76, 77 and 78 were tested in the above assay and found to have an IC₅₀ of less than 500 nM.

The compounds of Examples 2, 3, 4, 5, 6, 7, 8, 9, 10, 11, 12, 13, 14, 15, 16, 17, 18, 19, 20, 21, 23, 24, 25, 26, 27, 28, 29, 30, 31, 32, 33, 34, 35, 36, 37, 38, 39, 40, 42, 43, 44, 45, 46, 47, 48, 49, 50, 51, 52, 53, 54, 55, 56, 57, 58, 59, 60, 61, 62, 63, 64, 65, 66, 67, 69, 70, 71, 72, 73 and 74 were tested in the above assay and found to have an IC₅₀ of less than 100 nM.

The compounds of Examples 2, 3, 4, 5, 6, 7, 8, 9, 10, 11, 12, 13, 14, 15, 16, 17, 18, 19, 20, 21, 23, 24, 25, 26, 27, 28, 29, 30, 31, 32, 33, 34, 35, 36, 37, 38, 39, 40, 42, 43, 44, 45, 46, 47, 48, 49, 50, 51, 52, 53, 54, 55, 56, 57, 58, 59, 60, 61, 62, 63, 64, 65, 66, 67, 69, 70, 71, 72, 73, 74, 76, 77 and 78 were tested in the above assay and found to have an IC₅₀ of less than 100 nM.

The following compounds were tested in the above assay. Some compounds were prepared multiple times and tested in the above assay multiple times. The below data is representative of those tests:

| Example # | IC₅₀ (nM) |
|---|---|
| Example 1 | 429.46 |
| Example 2 | 55.84 |
| Example 3 | 0.44 |
| Example 4 | 0.62 |
| Example 5 | 1.06 |
| Example 6 | 2.52 |
| Example 7 | 4.76 |
| Example 8 | 5.83 |
| Example 9 | 1.17 |
| Example 10 | 2.29 |
| Example 11 | 7.03 |
| Example 12 | 1.85 |
| Example 13 | 1.91 |
| Example 14 | 0.93 |
| Example 15 | 2.63 |
| Example 16 | 1.71 |
| Example 17 | 11.09 |
| Example 18 | 0.82 |
| Example 19 | 2.04 |
| Example 20 | 3.48 |
| Example 21 | 72.42 |
| Example 22 | 584.94 |
| Example 23 | 4 |
| Example 24 | 64.88 |
| Example 25 | 12.26 |
| Example 26 | 2.41 |
| Example 27 | 2.95 |
| Example 28 | 0.84 |
| Example 29 | 0.65 |
| Example 30 | 0.74 |
| Example 31 | 0.56 |
| Example 32 | 34.88 |
| Example 33 | 17.17 |
| Example 34 | 4.83 |
| Example 35 | 2.1 |
| Example 36 | 37.45 |
| Example 37 | 3.8 |
| Example 38 | 15.1 |
| Example 39 | 64.17 |
| Example 40 | 27.02 |
| Example 41 | 151.99 |
| Example 42 | 1.76 |
| Example 43 | 2.63 |
| Example 44 | 0.64 |
| Example 45 | 0.53 |
| Example 46 | 6.09 |
| Example 47 | 4.14 |
| Example 48 | 1.68 |
| Example 49 | 3.91 |
| Example 50 | 1.22 |
| Example 51 | 2.01 |
| Example 52 | 70.43 |
| Example 53 | 13.19 |
| Example 54 | 3.36 |
| Example 55 | 1.47 |
| Example 56 | 1.31 |
| Example 57 | 26.03 |
| Example 58 | 37.07 |
| Example 59 | 10.59 |
| Example 60 | 1.28 |
| Example 61 | 80.09 |
| Example 62 | 1.45 |
| Example 63 | 1.71 |
| Example 64 | 9.29 |
| Example 65 | 0.63 |
| Example 66 | 1.5 |
| Example 67 | 61.01 |
| Example 68 | 658.18 |
| Example 69 | 1.66 |
| Example 70 | 25.66 |
| Example 71 | 0.66 |
| Example 72 | 0.4 |
| Example 73 | 0.86 |
| Example 74 | 0.51 |
| Example 75 | 138.7 |
| Example 76 | 13 |
| Example 77 | 0.56 |
| Example 78 | 5.19 |

### Biological Example 2

### Tumor Growth Inhibition (LOX)

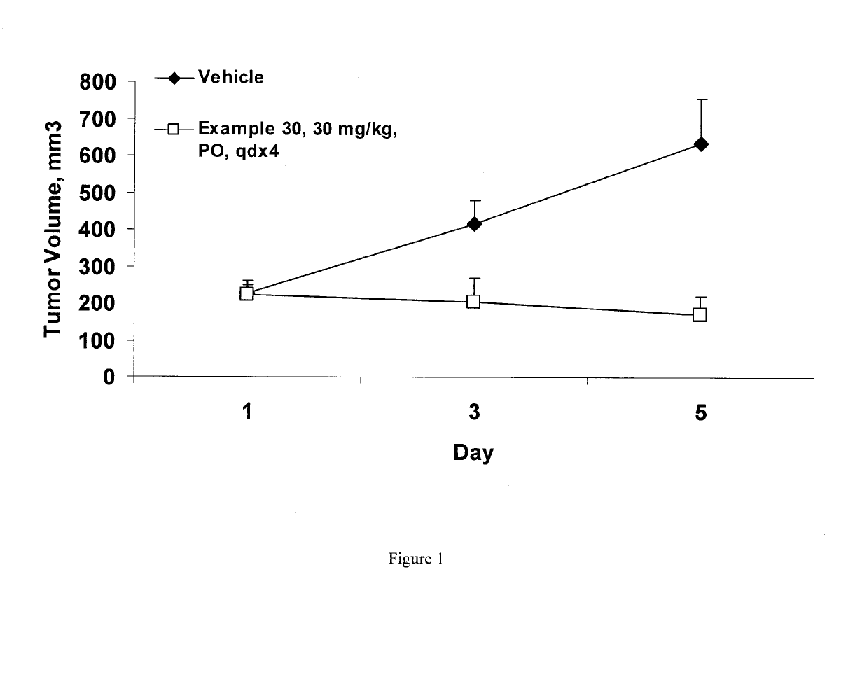

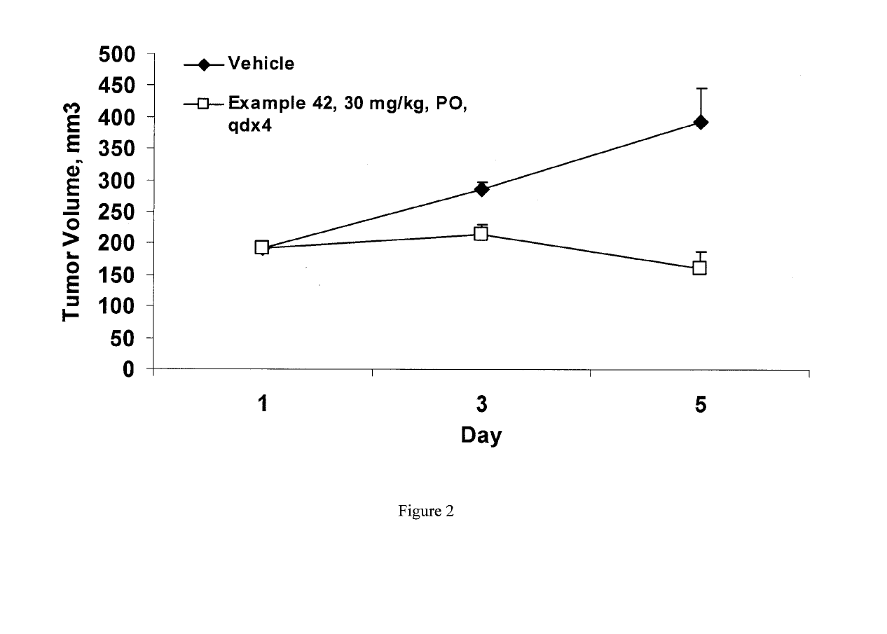

Female nude mice were implanted subcutaneously on the right flank with approximately 3.5×10⁶ LOX cells in 100 μL PBS. Five to seven days later, tumors were measured and mice were randomized into groups of six with average tumor volume in each group of approximately 200 mm³. Examples 30 and 42 were dissolved in 80% PEG400/20% ethanol before dosing, and administered PO at a volume of 5 mL/kg. Dosing was vehicle alone on days 1, 2, 3 and 4; and Examples 30 and 42 at 30 mg/kg on days 1, 2, 3 and 4 Animal weights and tumor volumes were measured using electronic calipers on day 5. Tumor volume was calculated using the formula: volume=(width²×length)/2. Each Example was run in a separate study with its own vehicle comparison. The results are shown in FIGS. 1, and 2.

| | Tumor Volume | Tumor Volume | Tumor Volume |
|---|---|---|---|
| | (mm³) | (mm³) | (mm³) |
| Compound | Day 1 | Day 3 | Day 5 |
| Vehicle | 225.46 | 415.11 | 632.52 |
| Example 30 | 221.68 | 204.49 | 168.94 |
| Vehicle | 192.52 | 284.96 | 393.63 |
| Example 42 | 191.22 | 214.61 | 161.55 |

### Intermediate Example 1

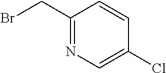

### 2-(bromomethyl)-5-chloropyridine

Step A: Ethyl 5-chloro-2-pyridinecarboxylate (104.0 mg, 0.560 mmol) was dissolved in THF (5.6 mL) and cooled to 0° C. The reaction mixture was then slowly treated with lithium aluminum hydride 1.0M solution in THF (0.392 μL, 0.392 mmol) and warmed to ambient temperature. The reaction mixture was allowed to stir at ambient temperature for 1 hour and then cooled to 0° C. The reaction mixture was then treated with water (15 μL) followed by 1.0N NaOH (15 μL) and then water (45 μL). The reaction mixture was warmed to ambient temperature and allowed to stir for 30 minutes. The reaction mixture was diluted with EtOAc, filtered through glass microfibre filter (“GF/F”) paper and concentrated. Silica gel chromatography eluting with a gradient of 50% hexanes/EtOAc to 100% hexanes/EtOAc provided (5-chloropyridin-2-yl)methanol (60.0 mg, 0.418 mmol, 74.6% yield).

Step B: (5-Chloropyridin-2-yl)methanol (40.0 mg, 0.279 mmol) was dissolved in dichloromethane (“DCM”) (2.8 mL) and treated with 1.0M phosphorus tribromide in DCM (0.31 μL, 0.306 mmol). The reaction mixture was allowed to stir at ambient temperature for 1 hour. The mixture was quenched with excess phosphorus tribromide with a few drops of water and stirred for 10 minutes. The mixture was diluted with EtOAc, washed with saturated NaHCO₃ (2×), brine (1×), dried over Na₂SO₄ and concentrated to afford 2-(bromomethyl)-5-chloropyridine (57.5 mg, 0.278 mmol, 100% yield).

### Example 1

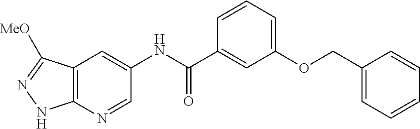

### 3-(benzyloxy)-N-(3-methoxy-1H-pyrazolo[3,4-b]pyridin-5-yl)benzamide

Step A: A suspension of 3-methoxy-1H-pyrazol-5-amine (0.84 g, 7.43 mmol; prepared as described in JP 01013072) and sodium nitromalonaldehyde monohydrate (1.23 g, 7.81 mmol) in water (40 mL) was heated to 90° C. for 16 hours. The reaction mixture was cooled to room temperature and poured into ethyl acetate (200 mL). The pH of the aqueous layer was adjusted to about 5 with acetic acid. The layers were separated, and the organic layer was dried, filtered and concentrated. The crude product was purified by column chromatography, eluting with hexanes/ethyl acetate (4:1) to give 3-methoxy-5-nitro-1H-pyrazolo[3,4-b]pyridine (0.625 g, 3.22 mmol, 43% yield) as a solid. ¹H NMR (400 MHz, (CD₃)₂SO) δ 13.46 (br s, 1H), 9.30 (s, 1H), 8.96 (s, 1H), 4.07 (s, 3H); m/z (APCI-neg) M−1=193.0.

Step B: 10% wt Pd/C (4.03, 3.8 mmol) was added to a solution of 3-methoxy-5-nitro-1H-pyrazolo[3,4-b]pyridine (7.3 g, 38.0 mmol) in ethyl acetate/MeOH (1:1, 240 mL). The reaction mixture was hydrogenated under 30 psi of hydrogen for 16 hours. The Pd/C was removed by filtration, and the filtrate was concentrated to give 3-methoxy-1H-pyrazolo[3,4-b]pyridin-5-amine (5.1 g, 31.1 mmol, 82% yield) as a solid. ¹H NMR (400 MHz, CD₃OD) δ 8.09 (d, J=2.5 Hz, 1H), 7.33 (d, J=2.5 Hz, 1H), 4.02 (s, 3H); m/z (APCI-pos) M+1=165.1.

Step C: 3-Methoxy-1H-pyrazolo[3,4-b]pyridin-5-amine (33 mg, 0.201 mmol) was dissolved in DMF (2 mL) and sequentially treated with 3-(benzyloxy)benzoic acid (50.5 mg, 0.221 mmol), 1-ethyl-(3-dimethylaminopropyl)carbodiimide hydrochloride, anhydrous (42.4 mg, 0.221 mmol), and 1-hydroxybenzotriazole (29.9 mg, 0.221 mmol) at ambient temperature. After 16 hours the reaction mixture was diluted with EtOAc and washed with water (4×), sodium bicarbonate (2×), and brine (1×), dried over sodium sulfate and concentrated. Silica gel chromatography eluting with a gradient of 3% methanol/DCM to 15% methanol/DCM gave 3-(benzyloxy)-N-(3-methoxy-1H-pyrazolo[3,4-b]pyridin-5-yl)benzamide (42.1 mg, 0.112 mmol, 55.9% yield) as a solid. ¹H NMR (400 MHz, (CD₃)₂SO) δ 12.49 (s, 1H), 10.40 (s, 1H), 8.72-8.72 (d, 1H), 8.46-8.45 (d, 1H), 7.63-7.57 (t, 2H), 7.49-7.35 (m, 6H), 7.27-7.25 (m, 1H), 5.20 (s, 2H), 4.01 (s, 3H); m/z (APCI-pos) M+1=375.2.

### Example 2

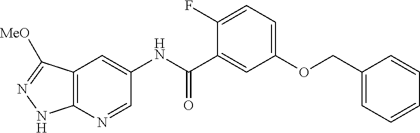

### 5-(benzyloxy)-2-fluoro-N-(3-methoxy-1H-pyrazolo[3,4-b]pyridin-5-yl)benzamide

Step A: 2-Fluoro-5-hydroxybenzoic acid (0.100 g, 0.642 mmol) was dissolved in DMF (5 mL) and cooled to 0° C. NaH (0.128 g, 60% wt, 3.20 mmol) was added, and the reaction was warmed to room temperature and stirred for 20 minutes. Benzyl bromide (0.229 mL, 1.92 mmol) was added, and the reaction was stirred at room temperature overnight. The reaction was partitioned between EtOAc and 0.1N HCl, and the layers were separated. The organic layer was extracted with 0.1N NaOH (2×), and the aqueous layer was acidified with 1N HCl to pH 2 and extracted with DCM (2×). The organic solution was dried over Na₂SO₄ and concentrated to give 5-(benzyloxy)-2-fluorobenzoic acid (0.104 g, 65.9%) as an oil, which was used directly in the next step.

Step B: 5-(Benzyloxy)-2-fluoro-N-(3-methoxy-1H-pyrazolo[3,4-b]pyridin-5-yl)benzamide (65.6%) was prepared according to the general procedure in Example 1, Step C substituting 5-(benzyloxy)-2-fluorobenzoic acid for 3-(benzyloxy)benzoic acid. ¹H NMR (400 MHz, (CD₃)₂SO) δ 12.55 (s, 1H), 10.58 (s, 1H), 8.66 (s, 1H), 8.48 (s, 1H), 7.46-7.48 (m, 2H), 7.40-7.43 (m, 2H), 7.32-7.36 (m, 3H), 7.21-7.23 (m, 1H), 5.17 (s, 2H), 4.02 (s, 3H); m/z (APCI-neg) M−1=391.0.

### Example 3

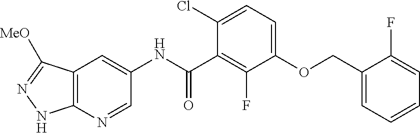

### 6-chloro-2-fluoro-3-(2-fluorobenzyloxy)-N-(3-methoxy-1H-pyrazolo[3,4-b]pyridin-5-yl)benzamide

Step A: 6-Chloro-2-fluoro-3-methoxybenzoic acid (1.83 g, 8.95 mmol) was dissolved in DCM (44.8 mL) and cooled to 0° C. Tribromoborane (26.9 mL, 1.0 M in DCM, 26.9 mmol) was added, and the reaction mixture was warmed to ambient temperature and allowed to stir for 3 hours. The reaction mixture was poured over ice and extracted with DCM. The organic layer was washed with 1.0N NaOH (2×), and the aqueous layer was acidified with 1.0N HCl to pH 2 and extracted with ethyl acetate (2×). The organic solution was washed with water (2×) and brine (1×) and dried over Na₂SO₄ and concentrated to give 6-chloro-2-fluoro-3-hydroxybenzoic acid (1.47 g, 7.70 mmol, 86.0% yield) as a solid. m/z (APCI-neg) M−1=188.6, 190.6.

Step B: 6-Chloro-2-fluoro-3-hydroxybenzoic acid (69.9 mg, 0.367 mmol) was dissolved in DMF (3.7 mL) and cooled to 0° C. NaH (44.0 mg, 60% wt, 1.10 mmol) was added, and the reaction was warmed to ambient temperature and stirred for 30 minutes. 1-(Bromomethyl)-2-fluorobenzene (48.7 μL, 0.404 mmol) was added, and the reaction was stirred at ambient temperature for 2 hours. The reaction was partitioned between EtOAc and 0.1N HCl, and the layers were separated. The organic layer was extracted with 0.1N NaOH (2×) and partitioned with hexanes (2×). The aqueous layer was acidified with 1N HCl to pH 3 and extracted with EtOAc (2×). The organic solution was washed with water (2×) and brine (1×), dried over Na₂SO₄, and concentrated to give 6-chloro-2-fluoro-3-(2-fluorobenzyloxy)benzoic acid (30.4 mg, 0.102 mmol, 27.8%). m/z (APCI-neg) M−1=296.5, 298.6.

Step C: 6-Chloro-2-fluoro-3-(2-fluorobenzyloxy)-N-(3-methoxy-1H-pyrazolo[3,4-b]pyridin-5-yl)benzamide (10.4 mg, 0.023 mmol, 23.0% yield) was prepared according to the general procedure in Example 1, Step C substituting 6-chloro-2-fluoro-3-(2-fluorobenzyloxy)benzoic acid (30.4 mg, 0.102 mmol) for 3-(benzyloxy)benzoic acid. ¹H NMR (400 MHz, (CD₃)₂SO) δ 12.59 (s, 1H), 11.03 (s, 1H), 8.59-8.58 (d, 1H), 8.47-8.46 (d, 1H), 7.61-7.56 (m, 1H), 7.50-7.34 (m, 3H), 7.31-7.25 (m, 2H), 5.30 (s, 2H), 4.01 (s, 3H); m/z (APCI-pos) M+1=445.1, 447.1.

### Example 4

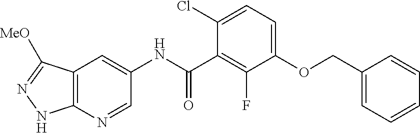

### 3-(benzyloxy)-6-chloro-2-fluoro-N-(3-methoxy-1H-pyrazolo[3,4-b]pyridin-5-yl)benzamide

Step A: 6-Chloro-2-fluoro-3-methoxybenzoic acid (5.25 g, 25.7 mmol) was dissolved in DCM (128 mL) and cooled to 0° C. Boron tribromide (77.0 mL, 1.0M in DCM, 77.0 mmol) was added, and the reaction mixture was warmed to ambient temperature and stirred for 3 hours. The reaction mixture was poured over ice and extracted with DCM. The organic layer was washed with 1.0N NaOH (2×), and the aqueous layer was acidified with 1.0N HCl to pH 2 and extracted with ethyl acetate (2×). The organic extract was washed with water (2×) and brine (1×), dried over Na₂SO₄ and concentrated to give 6-chloro-2-fluoro-3-hydroxybenzoic acid (4.71 g, 24.7 mmol, 96.3% yield) as a solid. m/z (APCI-neg) M−1=188.8, 190.8.

Step B: 6-Chloro-2-fluoro-3-hydroxybenzoic acid (185.0 mg, 0.971 mmol) was dissolved in MeOH (1.9 mL) and slowly treated with (trimethylsilyl)diazomethane (2.4 mL, 2.0M in hexanes, 4.85 mmol) over 10 minutes at ambient temperature. The reaction mixture was allowed to stir at ambient temperature for 10 minutes and was then concentrated. Silica gel chromatography eluting with a gradient of 5% EtOAc/Hexanes to 50% EtOAc/Hexanes gave methyl 6-chloro-2-fluoro-3-hydroxybenzoate (185 mg, 0.906 mmol, 93.3% yield). m/z (APCI-neg) M−1=203.1, 205.1.

Step C: Methyl 6-chloro-2-fluoro-3-hydroxybenzoate (185 mg, 0.906 mmol) was dissolved in DMF (2.2 mL) and sequentially treated with (bromomethyl)benzene (162 μL, 1.36 mmol) and potassium carbonate (376 mg, 2.72 mmol) and allowed to stir at ambient temperature for 1.5 hours. The reaction was acidified with 1.0N HCl to pH 7, extracted with EtOAc, washed with water (4×) and brine (1×), dried over Na₂SO₄ and concentrated. Silica gel chromatography eluting with a gradient of 5% EtOAc/hexanes to 50% EtOAc/hexanes gave methyl 3-(benzyloxy)-6-chloro-2-fluorobenzoate (178 mg, 0.604 mmol, 66.7% yield).

Step D: Methyl 3-(benzyloxy)-6-chloro-2-fluorobenzoate (178 mg, 0.604 mmol) was dissolved in 4:1 THF:MeOH (6.0 mL) and treated with 2.0M potassium hydroxide (1.5 mL, 3.02 mmol) and heated to 50° C. for 6 hours. After cooling to ambient temperature and acidification with 1.0N HCl to pH 4, the volatiles were removed, and the solution was extracted with EtOAc. The organic was washed with water (2×) and brine (1×), dried over Na₂SO₄ and concentrated to give 3-(benzyloxy)-6-chloro-2-fluorobenzoic acid (137 mg, 0.488 mmol, 80.8% yield). m/z (APCI-neg) M−1=278.6, 280.6.

Step E: 3-(Benzyloxy)-6-chloro-2-fluoro-N-(3-methoxy-1H-pyrazolo[3,4-b]pyridin-5-yl)benzamide (22.1%) was prepared according to the general procedure in Example 1, Step C, substituting 3-(benzyloxy)-6-chloro-2-fluorobenzoic acid (11.3 mg, 0.0403 mmol) for 3-(benzyloxy)benzoic acid. ¹H NMR (400 MHz, CD₃OD) δ 8.60-8.59 (d, 1H), 8.50-8.49 (d, 1H), 7.48-7.22 (m, 7H), 5.22 (s, 2H), 4.09 (s, 3H); m/z (APCI-pos) M+1=427.1, 429.1.

### Example 5

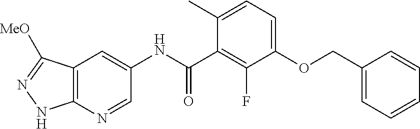

### 3-(benzyloxy)-2-fluoro-N-(3-methoxy-1H-pyrazolo[3,4-b]pyridin-5-yl)-6-methylbenzamide

Step A: 6-Bromo-2-fluoro-3-methoxy-benzoic acid (1.01 g, 4.07 mmol) was dissolved in MeOH (20.0 mL) and slowly treated with (trimethylsilyl)diazomethane (10.2 mL, 2.0M in hexanes, 20.4 mmol) over 10 minutes at ambient temperature. The reaction mixture was allowed to stir at ambient temperature for 10 minutes after complete addition of reagents and was then concentrated. Silica gel chromatography eluting with a gradient of 5% EtOAc/hexanes to 50% EtOAc/hexanes gave methyl 6-bromo-2-fluoro-3-methoxybenzoate (1.04 g, 3.94 mmol, 96.8% yield). ¹H NMR (400 MHz, (CD₃)₂SO) δ 7.53-7.50 (m, 1H), 7.31-7.27 (t, 1H), 3.91 (s, 3H), 3.88 (s, 3H).

Step B: Methyl 6-bromo-2-fluoro-3-methoxybenzoate (846 mg, 3.21 mmol) was dissolved in DMF (16.1 mL) and continually sparged with N₂ gas while treating with cesium carbonate (3.14 g, 9.64 mmol), dichloro[1,1′-bis(diphenylphosphino)ferrocene]palladium (II) dichloromethane adduct (264 mg, 0.321 mmol) and trimethylboroxine (444 mg, 3.54 mmol). The reaction was sparged with N₂ gas for an additional 5 minutes after complete addition of reagents and then heated to 115° C. for 16 hours. The reaction was cooled to ambient temperature, diluted with EtOAc, and filtered through GF/F paper. The solution was diluted with EtOAc, washed with water (4×) and brine (1×), dried over Na₂SO₄ and concentrated. Silica gel chromatography eluting with a gradient of 5% EtOAc/Hexanes to 50% EtOAc/Hexanes gave methyl 2-fluoro-3-methoxy-6-methylbenzoate (435 mg, 2.20 mmol, 68.3% yield). ¹H NMR (400 MHz, (CD₃)₂SO) δ 7.23-7.18 (t, 1H), 7.08-7.05 (d, 2H), 3.87 (s, 3H), 3.83 (s, 3H), 2.23 (s, 3H).

Step C: Methyl 2-fluoro-3-methoxy-6-methylbenzoate (435 mg, 2.20 mmol) was dissolved in 4:1 THF:MeOH (11.0 mL), treated with 2.0M potassium hydroxide (8.2 mL, 16.5 mmol) and heated to 50° C. for 16 hours. The reaction was cooled to ambient temperature and acidified with 1.0N HCl to pH 3. The volatiles were removed, and the aqueous was washed with water (2×) and brine (1×), dried over Na₂SO₄ and concentrated to give 2-fluoro-3-methoxy-6-methylbenzoic acid (278 mg, 1.51 mmol, 68.8% yield). ¹H NMR (400 MHz, (CD₃)₂SO) δ 13.49 (s, 1H), 7.16-7.12 (t, 1H), 7.04-7.01 (d, 1H), 3.82 (s, 3H), 2.24 (s, 3H).

Step D: 2-Fluoro-3-methoxy-6-methylbenzoic acid (278 mg, 1.51 mmol) was dissolved in DCM (10.0 mL) and cooled to 0° C. Boron tribromide (4.5 mL, 1.0M in DCM, 4.53 mmol) was added, and the reaction mixture was warmed to ambient temperature and allowed to stir for 2 hours. The reaction mixture was poured over ice and extracted with DCM. The organic layer was washed with 1.0N NaOH (2×), and the aqueous layer was acidified with 1.0N HCl to pH 2 and extracted with ethyl acetate (2×). The organic extract was washed with water (2×) and brine (1×), dried over Na₂SO₄ and concentrated to give 2-fluoro-3-hydroxy-6-methylbenzoic acid (220 mg, 1.30 mmol, 85.8% yield). m/z (APCI-neg) M−1=168.8.

Step E: 2-Fluoro-3-hydroxy-6-methylbenzoic acid (220 mg, 1.30 mmol) was dissolved in MeOH (6.5 mL) and slowly treated with (trimethylsilyl)diazomethane (3.2 mL, 2.0M in hexanes, 6.47 mmol) over 10 minutes at ambient temperature. The reaction mixture was allowed to stir at ambient temperature for 10 minutes after complete addition of reagents and then concentrated to methyl 2-fluoro-3-hydroxy-6-methylbenzoate (238 mg, 1.30 mmol, 100.0% yield). m/z (APCI-neg) M−1=183.0.

Step F: Methyl 2-fluoro-3-hydroxy-6-methylbenzoate (238 mg, 1.30 mmol) was dissolved in DMF (6.5 mL) and sequentially treated with (bromomethyl)benzene (231 μL, 1.94 mmol) and potassium carbonate (537 mg, 3.88 mmol) and allowed to stir at ambient temperature for 2 hours. The reaction was diluted with EtOAc, washed with water (4×) and brine (1×), dried over Na₂SO₄ and concentrated. Silica gel chromatography eluting with a gradient of 2% EtOAc/hexanes to 45% EtOAc/hexanes gave methyl 3-(benzyloxy)-2-fluoro-6-methylbenzoate (276 mg, 1.01 mmol, 77.7% yield). ¹H NMR (400 MHz, (CD₃)₂SO) δ 7.46-7.18 (m, 6H), 7.08-7.03 (t, 1H), 5.18 (s, 2H), 3.87 (s, 3H), 2.22 (s, 3H).

Step G: Methyl 3-(benzyloxy)-2-fluoro-6-methylbenzoate (276 mg, 1.01 mmol) was dissolved in 4:1 THF:MeOH (5.0 mL), treated with 2.0M potassium hydroxide (2.5 mL, 5.03 mmol) and heated to 50° C. for 16 hours. The reaction mixture was cooled to ambient temperature and acidified with 1.0N HCl to pH 3. The organic solvent was concentrated off, extracted with EtOAc, washed with water (2×) and brine (1×), dried over Na₂SO₄ and concentrated. C18 reversed phase chromatography eluting with a gradient of 5% acetonitrile/water to 95% acetonitrile/water gave 3-(benzyloxy)-2-fluoro-6-methylbenzoic acid (158 mg, 0.607 mmol, 60.3% yield). m/z (APCI-neg) M−1=258.8.

Step H: 3-(Benzyloxy)-2-fluoro-N-(3-methoxy-1H-pyrazolo[3,4-b]pyridin-5-yl)-6-methylbenzamide (60.8%) was prepared according to the general procedure in Example 1, Step C substituting 3-(benzyloxy)-2-fluoro-6-methylbenzoic acid (158 mg, 0.607 mmol) for 3-(benzyloxy)benzoic acid. ¹H NMR (400 MHz, (CD₃)₂SO) δ 12.55 (s, 1H), 10.81 (s, 1H), 8.62-8.61 (d, 1H), 8.50-8.49 (d, 1H), 7.48-7.35 (m, 5H), 7.27-7.23 (t, 1H), 7.07-7.05 (d, 1H), 5.21 (s, 2H), 4.01 (s, 3H), 2.26 (s, 3H); m/z (APCI-pos) M+1=407.1.

### Example 6

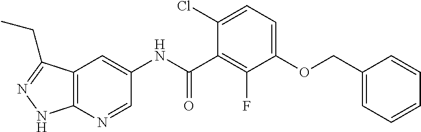

### 3-(benzyloxy)-6-chloro-N-(3-ethyl-1H-pyrazolo[3,4-b]pyridin-5-yl)-2-fluorobenzamide

Step A: 3-Ethyl-5-nitro-1H-pyrazolo[3,4-b]pyridine (52%) was prepared according to the general procedure in Example 1, Step A, substituting 3-ethyl-1H-pyrazol-5-amine for 3-methoxy-1H-pyrazol-5-amine.

Step B: 3-Ethyl-1H-pyrazolo[3,4-b]pyridine-5-amine (92%) was prepared according to the general procedure in Example 1, Step B, substituting 3-ethyl-5-nitro-1H-pyrazolo[3,4-b]pyridine for 3-methoxy-5-nitro-1H-pyrazolo[3,4-b]pyridine.

Step C: 3-(Benzyloxy)-6-chloro-N-(3-ethyl-1H-pyrazolo[3,4-b]pyridine-5-yl)-2-fluorobenzamide (66%) was prepared according to the general procedure in Example 1, Step C, substituting 3-ethyl-1H-pyrazolo[3,4-b]pyridin-5-amine for 3-methoxy-1H-pyrazolo[3,4-b]pyridin-5-amine and 3-(benzyloxy)-6-chloro-2-fluorobenzoic acid for 3-(benzyloxy)benzoic acid. ¹H NMR (400 MHz, (CD₃)₂SO) δ 13.23 (s, 1H), 11.01 (s, 1H), 8.63-8.62 (d, 1H), 8.56-8.56 (d, 1H), 7.49-7.35 (m, 7H), 5.28 (s, 2H), 2.96-2.91 (q, 2H), 1.34-1.30 (t, 3H); m/z (APCI-pos) M+1=425.1, 427.1.

### Example 7

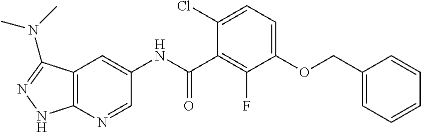

### 3-(benzyloxy)-6-chloro-N-(3-(dimethylamino)-1H-pyrazolo[3,4-b]pyridin-5-yl)-2-fluorobenzamide

Step A: A suspension of 1H-pyrazol-5-amine (0.804 g, 9.48 mmol) and sodium nitromalonaldehyde monohydrate (1.56 g, 9.96 mmol) in acetic acid (12 mL) was heated to 90° C. overnight. The reaction mixture was cooled to room temperature and poured into water (50 mL). The resulting solids were collected by filtration. The solids were washed with water (3×20 mL) and dried in vacuo to give 5-nitro-1H-pyrazolo[3,4-b]pyridine (1.40 g, 84%) as a solid.

Step B: 4N NaOH (5.12 mL, 20.5 mmol) was added to a cold (0° C.) solution of 5-nitro-1H-pyrazolo[3,4-b]pyridine (0.84 g, 5.12 mmol) in dioxane (30 mL), followed by bromine (1.05 mL, 20.5 mmol). The cold bath was removed, and the reaction mixture was left at room temperature for 30 minutes. The reaction mixture was diluted with ethyl acetate (100 mL) and quenched with saturated aqueous Na₂S₂O₃ (50 mL). The aqueous layer was extracted with ethyl acetate (100 mL). The combined organic layers were dried, filtered and concentrated. The crude product was purified by column chromatography, eluting with hexanes/ethyl acetate (9:1) to give 3-bromo-5-nitro-1H-pyrazolo[3,4-b]pyridine (1.10 g, 88%) as a solid.

Step C: 40% Dimethyl amine in water (2.6 mL, 21 mmol) was added to a solution of 3-bromo-5-nitro-1H-pyrazolo[3,4-b]pyridine (0.063 g, 0.26 mmol) in DMF (6.0 mL), and the mixture was placed in a microwave reactor at 140° C. for 15 hours. The reaction mixture was diluted with ethyl acetate (100 mL), and the organic layer was washed with water (3×50 mL). The organic layers were dried, filtered and concentrated. The crude product was purified by column chromatography, eluting with hexanes/ethyl acetate (4:1) to give N,N-dimethyl-5-nitro-1H-pyrazolo[3,4-b]pyridin-3-amine (0.012 g, 22%) as a solid.

Step D: N3,N3-dimethyl-1H-pyrazolo[3,4-b]pyridine-3,5-diamine (78%) was prepared according to Example 1, Step B, substituting N,N-dimethyl-5-nitro-1H-pyrazolo[3,4-b]pyridin-3-amine for 3-methoxy-5-nitro-1H-pyrazolo[3,4-b]pyridine.

Step E: 3-(Benzyloxy)-6-chloro-N-(3-(dimethylamino)-1H-pyrazolo[3,4-b]pyridin-5-yl)-2-fluorobenzamide (5%) was prepared according to the general procedure in Example 1, Step C, substituting N3,N3-dimethyl-1H-pyrazolo[3,4-b]pyridine-3,5-diamine for 3-methoxy-1H-pyrazolo[3,4-b]pyridin-5-amine and substituting 3-(benzyloxy)-6-chloro-2-fluorobenzoic acid for 3-(benzyloxy)benzoic acid. ¹H NMR (400 MHz, CD₃OD) δ 8.74-8.73 (d, 1H), 8.53-8.52 (d, 1H), 7.47-7.23 (m, 7H), 5.22 (s, 2H), 3.12 (s, 6H); m/z (APCI-pos) M+1=440.1, 442.1.

### Example 8

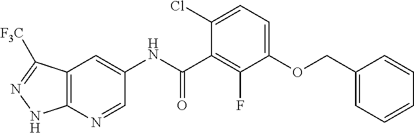

### 3-(benzyloxy)-6-chloro-2-fluoro-N-(3-(trifluoromethyl)-1H-pyrazolo[3,4-b]pyridin-5-yl)benzamide

Step A: Lithium diisopropylamide (8.2 mL, 14.8 mmol, 1.8M in heptane) was added to THF (20 mL) cooled to −78° C. in a dry ice/acetone bath. 2-Fluoropyridine (1.07 mL, 12.4 mmol) was added dropwise, and the resulting mixture was stirred at −78° C. for 3 hours. Ethyl trifluoroacetate (2.06 mL, 17.2 mmol) was added to the suspension dropwise. The reaction mixture was allowed to slowly warm to room temperature. After 1 hour, the mixture was poured into 1M hydrochloric acid (35 mL) and extracted twice with ethyl acetate. The combined ethyl acetate extracts were washed with brine, dried over magnesium sulfate, filtered, and evaporated to yield the hydrate of 2,2,2-trifluoro-1-(2-fluoropyridin-3-yl)ethanone (1.9 g, 90%) as a semisolid.

Step B: Hydrazine hydrate (3.06 mL, 63.0 mmol) was added to 2,2,2-trifluoro-1-(2-fluoropyridin-3-yl)ethanone (1.9 g, 9.0 mmol) in absolute ethanol (50 mL), and the mixture was heated to reflux overnight. The cooled reaction mixture was evaporated to afford a solid. The solid was partitioned between water and ethyl acetate. The aqueous layer was extracted with another portion of ethyl acetate. The combined ethyl acetate extracts were washed twice with brine, dried over magnesium sulfate, filtered, and evaporated to yield 3-(trifluoromethyl)-1H-pyrazolo[3,4-b]pyridine (1.43 g, 85%) as a solid.

Step C: Trifluoroacetic anhydride (2.6 mL, 18.7 mmol) was added to a solution of tetrabutylammonium nitrate (5.7 g, 18.7 mmol) in dichloromethane (50 mL) cooled to 0° C. in an ice bath. After 5 minutes, 3-(trifluoromethyl)-1H-pyrazolo[3,4-b]pyridine (0.5 g, 2.67 mmol) was added portionwise. The resulting solution was stirred at room temperature overnight. The reaction mixture was treated with saturated aqueous sodium bicarbonate, and the layers were separated. The aqueous layer was extracted with dichloromethane. The combined organic layers were washed with saturated aqueous sodium bicarbonate, dried over magnesium sulfate, filtered, and evaporated to an oil. The crude product was purified by column chromatography, eluting with hexanes/ethyl acetate (2:1) to give 5-nitro-3-(trifluoromethyl)-1H-pyrazolo[3,4-b]pyridine (0.19 g, 31%) as a solid.

Step D: SnCl₂.2H₂O (1.3 g, 5.7 mmol) was added to 5-nitro-3-(trifluoromethyl)-1H-pyrazolo[3,4-b]pyridine (0.19 g, 0.82 mmol) in ethyl acetate (20 mL). The resulting solution was heated to reflux for 3 hours. The cooled solution was treated with dilute aqueous sodium bicarbonate. The resulting slurry was filtered through Celite®, and the filter cake was washed with ethyl acetate. The layers were separated, and the organic layer was washed with brine, dried over magnesium sulfate, filtered, and evaporated to yield 3-(trifluoromethyl)-1H-pyrazolo[3,4-b]pyridine-5-amine (0.17 g, 99%) as a film.

Step E: 3-(Benzyloxy)-6-chloro-2-fluoro-N-(3-(trifluoromethyl)-1H-pyrazolo[3,4-b]pyridin-5-yl)benzamide (25%) was prepared according to the general procedure in Example 1, Step C, substituting 3-(trifluoromethyl)-1H-pyrazolo[3,4-b]pyridine-5-amine for 3-methoxy-1H-pyrazolo[3,4-b]pyridin-5-amine and substituting 3-(benzyloxy)-6-chloro-2-fluorobenzoic acid for 3-(benzyloxy)benzoic acid. ¹H NMR (400 MHz, CD₃OD) δ 8.75 (m, 2H), 7.46-7.23 (m, 7H), 5.21 (s, 2H); m/z (APCI-nega) M−1=463.2, 465.2.

### Example 9

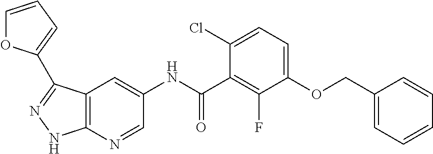

### 3-(benzyloxy)-6-chloro-2-fluoro-N-(3-(furan-2-yl)-1H-pyrazolo[3,4-b]pyridin-5-yl)benzamide

Step A: 3-(Furan-2-yl)-5-nitro-1H-pyrazolo[3,4-b]pyridine (21%) was prepared according to the general procedure in Example 1, Step A, substituting 3-(furan-2-yl)-1H-pyrazol-5-amine for 3-methoxy-1H-pyrazol-5-amine.

Step B: 3-(Furan-2-yl)-1H-pyrazolo[3,4-b]pyridin-5-amine (91%) was prepared according to the general procedure in Example 1, Step B, substituting 3-(furan-2-yl)-5-nitro-1H-pyrazolo[3,4-b]pyridine for 3-methoxy-5-nitro-1H-pyrazolo[3,4-b]pyridine.

Step C: 3-(Benzyloxy)-6-chloro-2-fluoro-N-(3-(furan-2-yl)-1H-pyrazolo[3,4-b]pyridin-5-yl)benzamide (45%) was prepared according to the general procedure in Example 1, Step C, substituting 3-(furan-2-yl)-1H-pyrazolo[3,4-b]pyridin-5-amine for 3-methoxy-1H-pyrazolo[3,4-b]pyridin-5-amine and substituting 3-(benzyloxy)-6-chloro-2-fluorobenzoic acid for 3-(benzyloxy)benzoic acid. ¹H NMR (400 MHz, (CD₃)₂SO) δ 13.89 (s, 1H), 11.15 (s, 1H), 8.96 (s, 1H), 8.68 (s, 1H), 7.92 (s, 1H), 7.38-7.49 (m, 7H), 6.96-6.97 (d, 1H), 6.69-6.70 (d, 1H), 5.28 (s, 2H); m/z (APCI-pos) M+1=463.1, 465.2.

### Example 10

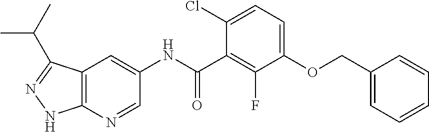

### 3-(benzyloxy)-6-chloro-2-fluoro-N-(3-isopropyl-1H-pyrazolo[3,4-b]pyridin-5-yl)benzamide

Step A: 3-Isopropyl-5-nitro-1H-pyrazolo[3,4-b]pyridine (45%) was prepared according to the general procedure in Example 1, Step A, substituting 3-isopropyl-1H-pyrazol-5-amine for 3-methoxy-1H-pyrazol-5-amine.

Step B: 3-Isopropyl-1H-pyrazolo[3,4-b]pyridin-5-amine (76%) was prepared according to the general procedure in Example 1, Step B, substituting 3-isopropyl-5-nitro-1H-pyrazolo[3,4-b]pyridine for 3-methoxy-5-nitro-1H-pyrazolo[3,4-b]pyridine.

Step C: 3-(Benzyloxy)-6-chloro-2-fluoro-N-(3-isopropyl-1H-pyrazolo[3,4-b]pyridin-5-yl)benzamide (30%) was prepared according to the general procedure in Example 1, Step C, substituting 3-isopropyl-1H-pyrazolo[3,4-b]pyridin-5-amine for 3-methoxy-1H-pyrazolo[3,4-b]pyridin-5-amine and substituting 3-(benzyloxy)-6-chloro-2-fluorobenzoic acid for 3-(benzyloxy)benzoic acid. ¹H NMR (400 MHz, (CD₃)₂SO) δ 13.24 (s, 1H), 11.02 (s, 1H), 8.68-8.67 (d, 1H), 8.60-8.59 (d, 1H), 7.50-7.37 (m, 7H), 5.28 (s, 2H), 3.39-3.32 (m, 1H), 1.39-1.37 (d, 6H); m/z (APCI-pos) M+1=439.1, 441.1.

### Example 11

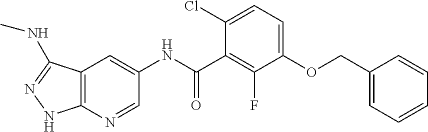

### 3-benzyloxy-6-chloro-2-fluoro-N-(3-(methylamino)-1H-pyrazolo[3,4-b]pyridin-5-yl)benzamide

Step A: Methylamine (33 wt. %) in absolute ethanol (952 μL, 7.64 mmol) was added to a solution of 3-bromo-5-nitro-1H-pyrazolo[3,4-b]pyridine (74.3 mg, 0.306 mmol) in water (1.0 mL), and the mixture was placed in a microwave reactor at 160° C. for 24 hours. The reaction mixture was diluted with ethyl acetate (100 mL), and the organic layer was washed with water (3×). The organic layers were dried, filtered and concentrated. The crude product was purified by column chromatography, eluting with hexanes/ethyl acetate to give N-methyl-5-nitro-1H-pyrazolo[3,4-b]pyridin-3-amine (0.012 g, 21%) as a solid.

Step B: N3-methyl-1H-pyrazolo[3,4-b]pyridine-3,5-diamine (20%) was prepared according to the general procedure in Example 1, Step B, substituting N-methyl-5-nitro-1H-pyrazolo[3,4-b]pyridin-3-amine for 3-methoxy-5-nitro-1H-pyrazolo[3,4-b]pyridine.

Step C: 3-(Benzyloxy)-6-chloro-2-fluoro-N-(3-(methylamino)-1H-pyrazolo[3,4-b]pyridin-5-yl)benzamide (38%) was prepared according to the general procedure in Example 1, Step C, substituting N3-methyl-1H-pyrazolo[3,4-b]pyridine-3,5-diamine for 3-methoxy-1H-pyrazolo[3,4-b]pyridin-5-amine and substituting 3-(benzyloxy)-6-chloro-2-fluorobenzoic acid for 3-(benzyloxy)benzoic acid. ¹H NMR (400 MHz, CD₃OD) δ 8.62-8.61 (d, 1H), 8.46-8.45 (d, 1H), 7.47-7.45 (m, 2H), 7.41-7.25 (m, 5H), 5.22 (s, 2H), 2.97 (s, 3H); m/z (APCI-pos) M+1=426.1, 428.1.

### Example 12

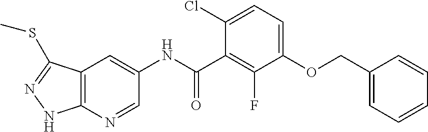

### 3-(benzyloxy)-6-chloro-2-fluoro-N-(3-(methylthio)-1H-pyrazolo[3,4-b]pyridin-5-yl)benzamide

Step A: Ethyl 2-cyanoacetate (2.00 mL, 18.7 mmol) was added dropwise to a cold suspension (0° C.) of NaH (1.50 g, 37.5 mmol, 60% in mineral oil) in benzene (20 mL), followed by the addition of CS₂ (1.7 mL, 28.1 mmol). DMF (4 mL) was added slowly. The mixture was stirred for 30 minutes before MeI (3.52 mL, 56.2 mmol) was added. The resulting mixture was stirred at room temperature overnight. Benzene (50 mL) was added, and the slurry was quenched with ice-water. The organic layer was separated, dried, filtered and concentrated. The crude product was purified by column chromatography, eluting with hexanes/ethyl acetate (4:1) to give ethyl 2-cyano-3,3-bis(methylthio)acrylate (2.2 g, 54%) as a solid.

Step B: A solution of ethyl 2-cyano-3,3-bis(methylthio)acrylate (2.2 g, 10.1 mmol) and hydrazine (0.325 mL, 10.1 mmol) in 2-propanol (20 mL) was heated at reflux overnight. The reaction mixture was cooled to room temperature and concentrated. The crude product was purified by column chromatography, eluting with hexanes/ethyl acetate (1:1) to give ethyl 5-amino-3-(methylthio)-1H-pyrazole-4-carboxylate (1.2 g, 59%) as a solid. m/z (APCI-pos) M+1=202.0.

Step C: Ethyl 5-amino-3-(methylthio)-1H-pyrazole-4-carboxylate (1.2 g, 5.96 mmol) was dissolved in a solution of LiOH (1.14 g, 47.7 mmol) in MeOH/H₂O (40 mL, 9:1). The resulting solution was heated at reflux for 72 hours. The reaction mixture was cooled to room temperature and concentrated. The residue was diluted with water, and the insoluble material was removed by filtration. The filtrate was extracted with ethyl acetate (4×100 mL), and the combined organic layers were dried, filtered and concentrated to give 3-(methylthio)-1H-pyrazol-5-amine (0.61 g, 79%) as a solid. m/z (APCI-pos) M+1=130.0.

Step D: 3-(Methylthio)-5-nitro-1H-pyrazolo[3,4-b]pyridine (86%) was prepared according to the general procedure in Example 1, Step A, substituting 3-(methylthio)-1H-pyrazol-5-amine for 3-methoxy-1H-pyrazol-5-amine.

Step E: 3-(Methylthio)-1H-pyrazolo[3,4-b]pyridin-5-amine (96%) was prepared according to the general procedure in Example 1, Step B, substituting 3-(methylthio)-5-nitro-1H-pyrazolo[3,4-b]pyridine for 3-methoxy-5-nitro-1H-pyrazolo[3,4-b]pyridine.

Step F: 3-(Benzyloxy)-6-chloro-2-fluoro-N-(3-(methylthio)-1H-pyrazolo[3,4-b]pyridin-5-yl)benzamide (61%) was prepared according to the general procedure in Example 1, Step C, substituting 3-(methylthio)-1H-pyrazolo[3,4-b]pyridin-5-amine for 3-methoxy-1H-pyrazolo[3,4-b]pyridin-5-amine and substituting 3-(benzyloxy)-6-chloro-2-fluorobenzoic acid for 3-(benzyloxy)benzoic acid. ¹H NMR (400 MHz, CD₃OD) δ 8.64 (s, 1H), 8.60 (s, 1H), 7.47-7.24 (m, 7H), 5.21 (s, 2H), 2.63 (s, 3H); m/z (APCI-neg) M−1=441.0, 443.0.

### Example 13

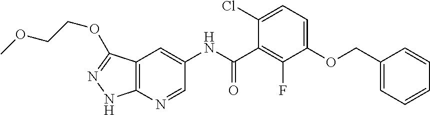

### 3-(benzyloxy)-6-chloro-2-fluoro-N-(3-(2-methoxyethoxy)-1H-pyrazolo[3,4-b]pyridin-5-yl)benzamide

Step A: Diisopropyl azodicarboxylate (5.05 g, 23.7 mmol) was added dropwise over a period of 15 minutes (internal temp<15° C.) to a cold (0° C.) solution of 5-amino-1H-pyrazol-3-ol (2.0 g, 19.8 mmol) and PPh₃ (6.23 g, 23.7 mmol) in DCM (30 mL). After stirring at 0° C. for 1 hour, 2-methoxyethanol (1.81 g, 23.7 mmol) was added dropwise over 10 minutes.

The reaction mixture was allowed to warm up to room temperature over 1 hour and stirred under N₂ for 3 days. The solids were removed by filtration, and the filter cake was washed with DCM. The DCM later was extracted with 1N HCl (2×50 mL). The combined aqueous layer was washed with DCM (100 mL), and the DCM layer was discarded. The aqueous layer was basified to about pH 8 with 2N NaOH and extracted with ethyl acetate (200 mL×3). The combined organics were dried, filtered and concentrated. The crude product was purified on by flash chromatography, eluting with ethyl acetate/MeOH (50:1) to give 3-(2-methoxyethoxy)-1H-pyrazol-5-amine (0.40 g, 2.55 mmol, 13% yield) as an oil. m/z (APCI-pos) M+1=158.2.

Step B: 3-(2-Methoxyethoxy)-5-nitro-1H-pyrazolo[3,4-b]pyridine (11%) was prepared according to the general procedure in Example 1, Step A, substituting 3-(2-methoxyethoxy)-1H-pyrazol-5-amine for 3-methoxy-1H-pyrazol-5-amine.

Step C: 3-(2-Methoxyethoxy)-1H-pyrazolo[3,4-b]pyridin-5-amine (100%) was prepared according to the general procedure in Example 1, Step B, substituting 3-(2-methoxyethoxy)-5-nitro-1H-pyrazolo[3,4-b]pyridine for 3-methoxy-5-nitro-1H-pyrazolo[3,4-b]pyridine.

Step D: 3-(Benzyloxy)-6-chloro-2-fluoro-N-(3-(2-methoxyethoxy)-1H-pyrazolo[3,4-b]pyridin-5-yl)benzamide (53%) was prepared according to the general procedure in Example 1, Step C, substituting 3-(2-methoxyethoxy)-1H-pyrazolo[3,4-b]pyridin-5-amine for 3-methoxy-1H-pyrazolo[3,4-b]pyridin-5-amine and substituting 3-(benzyloxy)-6-chloro-2-fluorobenzoic acid for 3-(benzyloxy)benzoic acid. ¹H NMR (400 MHz, (CD₃)₂SO) δ 12.60 (s, 1H), 11.04 (s, 1H), 8.56-8.55 (d, 1H), 8.51-8.51 (d, 1H), 7.49-7.36 (m, 7H), 5.28 (s, 2H), 4.46-4.44 (m, 2H), 3.75-3.73 (m, 2H), 3.33 (s, 3H); m/z (APCI-pos) M+1=471.2, 473.1.

### Example 14

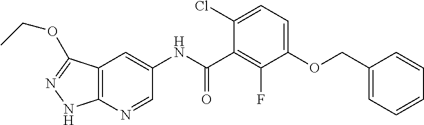

### 3-(benzyloxy)-6-chloro-N-(3-ethoxy-1H-pyrazolo[3,4-b]pyridin-5-yl)-2-fluorobenzamide

Step A: A solution of malononitrile (10.0 g, 151 mmol), ethanol (6.97 g, 151 mmol) and ether (120 mL) was cooled to 0° C., and 2.0M HCl in ether (98.4 mL, 197 mmol) was added rapidly via an addition funnel. The reaction mixture was stirred at room temperature for 16 hours. The solids were collected by filtration and washed with ether (100 mL) to give ethyl 2-cyanoacetimidate hydrochloride (12.6 g, 56%).

Step B: A solution of ethyl 2-cyanoacetimidate hydrochloride (12.6 g, 84.8 mmol) and hydrazine (3.67 g, 114 mmol) in EtOH (50 mL) was refluxed for 16 hours. The reaction mixture was concentrated, and the residue was taken up in water (100 mL), ethyl acetate (500 mL) and placed in an ice bath. 2N NaOH (˜6 mL) was added to this solution until the pH was adjusted to ˜7. The solids were removed by filtration (discarded), and the filtrate was transferred to a separation funnel. The layers were separated, and the aqueous layer was extracted with ethyl acetate (200 mL). The combined organics were dried, filtered and concentrated. The crude product was purified by flash chromatography, eluting with hexanes/ethyl acetate (1:1), hexanes/ethyl acetate (1:2) to give 3-ethoxy-1H-pyrazol-5-amine (1.15 g, 9.04 mmol, 11% yield) as a solid. m/z (APCI-pos) M+1=128.1.

Step C: 3-Ethoxy-5-nitro-1H-pyrazolo[3,4-b]pyridine (23%) was prepared according to the general procedure in Example 1, Step A, substituting 3-ethoxy-1H-pyrazol-5-amine for 3-methoxy-1H-pyrazol-5-amine.

Step D: 3-Ethoxy-1H-pyrazolo[3,4-b]pyridin-5-amine (72%) was prepared according to the general procedure in Example 1, Step B, substituting 3-ethoxy-5-nitro-1H-pyrazolo[3,4-b]pyridine for 3-methoxy-5-nitro-1H-pyrazolo[3,4-b]pyridine.

Step E: 3-(Benzyloxy)-6-chloro-N-(3-ethoxy-1H-pyrazolo[3,4-b]pyridin-5-yl)-2-fluorobenzamide (41%) was prepared according to the general procedure in Example 1, Step C, substituting 3-ethoxy-1H-pyrazolo[3,4-b]pyridin-5-amine for 3-methoxy-1H-pyrazolo[3,4-b]pyridin-5-amine and substituting 3-(benzyloxy)-6-chloro-2-fluorobenzoic acid for 3-(benzyloxy)benzoic acid. ¹H NMR (400 MHz, (CD₃)₂SO) δ 12.56 (s, 1H), 11.03 (s, 1H), 8.56-8.56 (d, 1H), 8.50-8.49 (d, 1H), 7.49-7.35 (m, 7H), 5.28 (s, 2H), 4.42-4.36 (q, 2H), 1.43-1.40 (t, 3H); m/z (APCI-pos) M+1=441.1, 443.1.

### Example 15

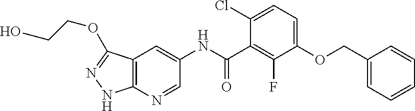

### 3-(benzyloxy)-6-chloro-2-fluoro-N-(3-(2-hydroxyethoxy)-1H-pyrazolo[3,4-b]pyridine-5-yl)benzamide

Step A: 1M NaOH (46.5 mL, 46.5 mmol) was added to a solution of ethyl 5-amino-3-(2-hydroxyethoxy)-1H-pyrazole-4-carboxylate (2.00 g, 9.29 mmol, prepared as described in Neidlein, Richard, et al. “Heterocyclic Compounds from 2-(Alkoxycarbonylcyanomethylene)-1,3-dioxolanes.” J. Het. Chem. Vol. 26 (1989): pp. 1335-1340) in ethanol (30 mL), and the mixture was refluxed overnight. The solution was washed with DCM with 25% isopropyl alcohol (“IPA”) and then acidified to pH 3 with concentrated HCl. Gas evolution was observed. The solution was washed with DCM with 25% IPA, and the aqueous layer was evaporated. The residue was treated with methanol, filtered, and the filtrate was evaporated to yield crude 2-(5-amino-1H-pyrazol-3-yloxy)ethanol (3.28 g) along with inorganic salts. m/z (APCI-pos) M+1=144.0.

Step B: 2-(5-Nitro-1H-pyrazolo[3,4-b]pyridin-3-yloxy)ethanol (8.5%) was prepared according to the general procedure in Example 1, Step A, substituting 2-(5-amino-1H-pyrazol-3-yloxy)ethanol for 3-methoxy-1H-pyrazol-5-amine.

Step C: 2-(5-Amino-1H-pyrazolo[3,4-b]pyridin-3-yloxy)ethanol (100%) was prepared according to the general procedure in Example 1, Step B, substituting 2-(5-nitro-1H-pyrazolo[3,4-b]pyridin-3-yloxy)ethanol for 3-methoxy-5-nitro-1H-pyrazolo[3,4-b]pyridine.

Step D: 3-(Benzyloxy)-6-chloro-2-fluoro-N-(3-(2-hydroxyethoxy)-1H-pyrazolo[3,4-b]pyridin-5-yl)benzamide (55%) was prepared according to the general procedure in Example 1, Step C, substituting 2-(5-amino-1H-pyrazolo[3,4-b]pyridin-3-yloxy)ethanol for 3-methoxy-1H-pyrazolo[3,4-b]pyridin-5-amine and substituting 3-(benzyloxy)-6-chloro-2-fluorobenzoic acid for 3-(benzyloxy)benzoic acid. ¹H NMR (400 MHz, CD₃OD) δ 8.59-8.56 (m, 2H), 7.46-7.25 (m, 7H), 5.21 (s, 2H), 4.47-4.45 (t, 2H), 3.97-3.95 (t, 2H); m/z (APCI-pos) M+1=457.1, 459.1.

### Example 16

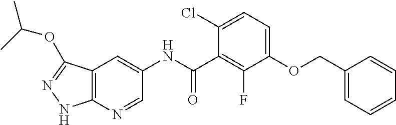

### 3-(benzyloxy)-6-chloro-2-fluoro-N-(3-isopropoxy-1H-pyrazolo[3,4-b]pyridin-5-yl)benzamide

Step A: 3-Isopropoxy-5-nitro-1H-pyrazolo[3,4-b]pyridine (52%) was prepared according to the general procedure in Example 1, Step A substituting 3-isopropoxy-1H-pyrazol-5-amine for 3-methoxy-1H-pyrazol-5-amine.

Step B: 3-Isopropoxy-1H-pyrazolo[3,4-b]pyridin-5-amine (96%) was prepared according to the general procedure in Example 1, Step B, substituting 3-isopropoxy-5-nitro-1H-pyrazolo[3,4-b]pyridine for 3-methoxy-5-nitro-1H-pyrazolo[3,4-b]pyridine.

Step C: 3-(Benzyloxy)-6-chloro-2-fluoro-N-(3-isopropoxy-1H-pyrazolo[3,4-b]pyridin-5-yl)benzamide (27%) was prepared according to the general procedure in Example 1, Step C, substituting 3-isopropoxy-1H-pyrazolo[3,4-b]pyridine-5-amine for 3-methoxy-1H-pyrazolo[3,4-b]pyridine-5-amine and substituting 3-(benzyloxy)-6-chloro-2-fluorobenzoic acid for 3-(benzyloxy)benzoic acid. ¹H NMR (400 MHz, (CD₃)₂SO) δ 12.55 (s, 1H), 11.03 (s, 1H), 8.54-8.53 (d, 1H), 8.50-8.49 (d, 1H), 7.49-7.36 (m, 7H), 5.28 (s, 2H), 5.09-5.03 (m, 1H), 1.41-1.39 (d, 6H); m/z (APCI-pos) M+1=455.1, 457.1.

### Example 17

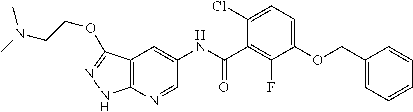

### 3-(benzyloxy)-6-chloro-N-(3-(2-(dimethylamino)ethoxy)-1H-pyrazolo[3,4-b]pyridin-5-yl)-2-fluorobenzamide

Step A: Nitromalonaldehyde sodium salt monohydrate (3.57 g, 22.7 mmol) was added to suspension of 5-amino-1-tosyl-1H-pyrazol-3-ol (5.00 g, 19.7 mmol; see Elgemeie, Galal H., et al. “Novel Synthesis of 5-Amino-1-arylsulfonyl-4-pyrazolin-3-ones as a New Class of N-Sulfonylated Pyrazoles.” J. Chem. Res. (S). Issue 6 (1999): pp. 384-385) in acetic acid (30 mL). The mixture was heated at 50° C. for 4 hours. The partial suspension was allowed to cool and diluted with water. The resulting solid was collected by vacuum filtration and dried under high vacuum to afford 5-nitro-1-tosyl-1H-pyrazolo[3,4-b]pyridin-3-ol (4.21 g, 12.6 mmol, 63.8% yield) as a solid.

Step B: 5-Nitro-1-tosyl-1H-pyrazolo[3, 4-1)]pyridin-3-ol (207 mg, 0.619 mmol) was dissolved in THF (6.2 ml) and treated with 2-(dimethylamino)ethanol (74.67 μL, 0.743 mmol) and triphenylphosphine (357 mg, 1.36 mmol). The reaction mixture was cooled to 0° C. and treated with diethyl azodicarboxylate (214 μL, 1.36 mmol) and allowed to warm to ambient temperature and stir for 16 hours. The solution was diluted with EtOAc, washed with sodium bicarbonate (2×) and brine (1×), dried over sodium sulfate, and concentrated. C18 reversed phase chromatography eluting with a gradient of 5% acetonitrile/water to 95% acetonitrile/water gave N,N-dimethyl-2-(5-nitro-1-tosyl-1H-pyrazolo[3,4-b]pyridin-3-yloxy)ethanamine (178 mg, 0.439 mmol, 71.0% yield). m/z (APCI-pos) M+1=406.0.

Step C: N,N-Dimethyl-2-(5-nitro-1-tosyl-1H-pyrazolo[3,4-b]pyridin-3-yloxy)ethanamine (178 mg, 0.439 mmol) was dissolved in MeOH (4.40 mL) and treated with aqueous 0.5M solution of potassium carbonate (4.40 mL, 2.20 mmol) and heated to 50° C. for 1 hour. The reaction mixture was concentrated, diluted with EtOAc, washed with sodium bicarbonate (2×) and brine (1×), dried over sodium sulfate, and concentrated to afford N,N-dimethyl-2-(5-nitro-1H-pyrazolo[3,4-b]pyridin-3-yloxy)ethanamine (77.1 mg, 0.307 mmol, 69.9% yield). m/z (APCI-neg) M−1=250.0.

Step D: 3-(2-(Dimethylamino)ethoxy)-1H-pyrazolo[3,4-b]pyridin-5-amine (100%) was prepared according to the general procedure in Example 1, Step B, substituting N,N-dimethyl-2-(5-nitro-1H-pyrazolo[3,4-b]pyridin-3-yloxy)ethanamine for 3-methoxy-5-nitro-1H-pyrazolo[3,4-b]pyridine.

Step E: 3-(Benzyloxy)-N-(3-(2-(dimethylamino)ethoxy)-1H-pyrazolo[3,4-b]pyridin-5-yl)-2,6-difluorobenzamide (26%) was prepared according to the general procedure in Example 1, Step C, substituting 3-(2-(dimethylamino)ethoxy)-1H-pyrazolo[3,4-b]pyridin-5-amine for 3-methoxy-1H-pyrazolo[3,4-b]pyridine-5-amine and substituting 3-(benzyloxy)-6-chloro-2-fluorobenzoic acid for 3-(benzyloxy)benzoic acid. ¹H NMR (400 MHz, (CD₃)₂SO) δ 12.61 (s, 1H), 11.05 (s, 1H), 8.55-8.53 (m, 2H), 7.49-7.36 (m, 7H), 5.28 (s, 2H), 4.47-4.45 (t, 2H), 2.88-2.86 (t, 2H), 2.35 (s, 6H); m/z (APCI-pos) M+1=484.1, 485.1.

### Example 18

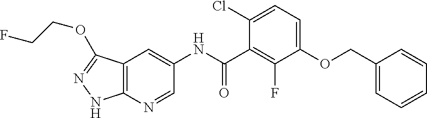

### 3-(benzyloxy)-6-chloro-2-fluoro-N-(3-(2-fluoroethoxy)-1H-pyrazolo[3,4-b]pyridine-5-yl)benzamide

Step A: 3-(2-Fluoroethoxy)-5-nitro-1-tosyl-1H-pyrazolo[3,4-b]pyridine was prepared according to the general procedure in Example 17, Step B, substituting 2-fluoroethanol for 2-(dimethylamino)ethanol and was used in Step B without further purification.

Step B: 3-(2-Fluoroethoxy)-5-nitro-1H-pyrazolo[3,4-b]pyridine was prepared according to the general procedure in Example 17, Step C, substituting 3-(2-fluoroethoxy)-5-nitro-1-tosyl-1H-pyrazolo[3,4-b]pyridine for N,N-dimethyl-2-(5-nitro-1-tosyl-1H-pyrazolo[3,4-b]pyridin-3-yloxy)ethanamine and was used in Step C without further purification.

Step C: 3-(2-Fluoroethoxy)-1H-pyrazolo[3,4-b]pyridin-5-amine (42% over three steps) was prepared according to the general procedure in Example 1, Step B, substituting 3-(2-fluoroethoxy)-5-nitro-1H-pyrazolo[3,4-b]pyridine for 3-methoxy-5-nitro-1H-pyrazolo[3,4-b]pyridine.

Step D: 3-(Benzyloxy)-6-chloro-2-fluoro-N-(3-(2-fluoroethoxy)-1H-pyrazolo[3,4-b]pyridine-5-yl)benzamide (37%) was prepared according to the general procedure in Example 1, Step C, substituting 3-(2-fluoroethoxy)-1H-pyrazolo[3,4-b]pyridine-5-amine for 3-methoxy-1H-pyrazolo[3,4-b]pyridine-5-amine and substituting 3-(benzyloxy)-6-chloro-2-fluorobenzoic acid for 3-(benzyloxy)benzoic acid. ¹H NMR (400 MHz, (CD₃)₂SO) δ 12.67 (s, 1H), 11.06 (s, 1H), 8.59-8.58 (d, 1H), 8.54-8.53 (d, 1H), 7.49-7.35 (m, 7H), 5.28 (s, 2H), 4.91-4.89 (m, 1H), 4.79-4.77 (m, 1H), 4.64-4.62 (m, 1H), 4.56-4.54 (m, 1H); m/z (APCI-pos) M+1=459.1, 461.1.

### Example 19

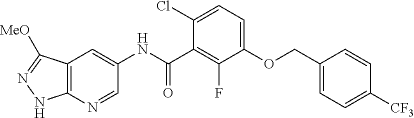

### 6-chloro-2-fluoro-N-(3-methoxy-1H-pyrazolo[3,4-b]pyridin-5-yl)-3-(4-(trifluoromethyl)benzyloxy)benzamide

Step A: 1-Ethyl-(3-dimethylaminopropyl)carbodiimide hydrochloride (5.18 g, 27.0 mmol) was added to a solution of 3-methoxy-1H-pyrazolo[3,4-b]pyridin-5-amine (3.7 g, 22.5 mmol) and 6-chloro-2-fluoro-3-hydroxybenzoic acid (5.15 g, 27.0 mmol) in MeCN (100 mL). The reaction mixture was heated to 50° C. for 16 hours. The reaction mixture was cooled to room temperature and concentrated to remove MeCN. The resulting solids were washed with water (500 mL), DCM (500 mL) and ethyl acetate (200 mL). The remaining solids were dried under vacuo to give 6-chloro-2-fluoro-3-hydroxy-N-(3-methoxy-1H-pyrazolo[3,4-b]pyridin-5-yl)benzamide (2.7 g, 8.02 mmol, 36% yield) as a solid. ¹H NMR (400 MHz, (CD₃)₂SO) δ 12.58 (br s, 1H), 10.98 (br s, 1H), 10.50 (br s, 111), 8.60 (s, 1′-1), 8.48 (s, 1H), 7.20 (m, 1H), 7.08 (m, 1H), 4.02 (s, 3H); m/z (APCI-pos) M+1=337.1, 339.1.

Step B: 6-Chloro-2-fluoro-3-hydroxy-N-(3-methoxy-1H-pyrazolo[3,4-b]pyridin-5-yl)benzamide (7.2 mg, 0.0214 mmol) was dissolved in THF and treated with (4-(trifluoromethyl)phenyl)methanol (3.22 μL, 0.0235 mmol) and triphenylphosphine (6.17 mg, 0.0235 mmol). The reaction was cooled to 0° C., treated with diethyl azodicarboxylate (7.4 μL, 0.0471 mmol) and allowed to stir at room temperature for 16 hours. The reaction was diluted with EtOAc, washed with water (2×) and brine (1×), dried over Na₂SO₄ and concentrated. The crude product was purified by column chromatography, eluting with hexanes/ethyl acetate (40% to 100% EtOAc/hexanes) to give 6-chloro-2-fluoro-N-(3-methoxy-1H-pyrazolo[3,4-b]pyridin-5-yl)-3-(4-(trifluoromethyl)benzyloxy)benzamide (4.0 mg, 38%) as a solid. ¹H NMR (400 MHz, (CD₃)₂SO) δ 12.59 (s, 1H), 11.03 (s, 1H), 8.59-8.58 (d, 1H), 8.48-8.47 (d, 1H), 7.82-7.79 (d, 2H), 7.71-7.68 (d, 21-1), 7.41-7.39 (m, 2H), 5.40 (s, 2H), 4.02 (s, 3H); m/z (APCI-pos) M+1=495.1, 497.1.

### Example 20

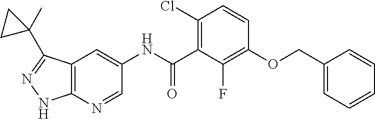

### 3-(benzyloxy)-6-chloro-2-fluoro-N-(3-(1-methylcyclopropyl)-1H-pyrazolo[3,4-b]pyridin-5-yl)benzamide

Step A: 3-(1-Methyl-cyclopropyl)-3-oxo-propionitrile (1.0 g, 8.12 mmol) was dissolved in EtOH (10 mL). Hydrazine monohydrate (3.03 mL, 40.6 mmol) was added, and the reaction mixture was heated to 80° C. for 30 minutes and then cooled to room temperature. The reaction was concentrated and purified by column chromatography eluting with EtOAc to give 3-(1-methylcyclopropyl)-1H-pyrazol-5-amine as an oil (311 mg, 28% yield).

Step B: 3-(1-Methylcyclopropyl)-5-nitro-1H-pyrazolo[3,4-b]pyridine (9%) was prepared according to the general procedure in Example 1, Step A, substituting 3-(1-methylcyclopropyl)-1H-pyrazol-5-amine for 3-methoxy-1H-pyrazol-5-amine.

Step C: 3-(1-Methylcyclopropyl)-1H-pyrazolo[3,4-b]pyridin-5-amine (96%) was prepared according to the general procedure in Example 1, Step B, substituting 3-(1-methylcyclopropyl)-5-nitro-1H-pyrazolo[3,4-b]pyridine for 3-methoxy-5-nitro-1H-pyrazolo[3,4-b]pyridine.

Step D: 3-(Benzyloxy)-6-chloro-2-fluoro-N-(3-(1-methylcyclopropyl)-1H-pyrazolo[3,4-b]pyridin-5-yl)benzamide (36%) was prepared according to the general procedure in Example 1, Step C, substituting 3-(1-methylcyclopropyl)-1H-pyrazolo[3,4-b]pyridin-5-amine for 3-methoxy-1H-pyrazolo[3,4-b]pyridin-5-amine and substituting 3-(benzyloxy)-6-chloro-2-fluorobenzoic acid for 3-(benzyloxy)benzoic acid. 1H NMR (400 MHz, (CD3)₂SO) δ 13.21 (s, 1H), 11.01 (s, 1H), 8.63 (s, 1H), 8.60 (s, 1H), 7.36-7.49 (m, 7H), 5.28 (s, 2H), 1.56 (s, 3H), 1.12-1.15 (m, 2H), 0.83-0.86 (m, 2H); m/z (APCI-pos) M+1=451.1, 453.2.

The following compounds in Table 1 were prepared according to the Example Number given in the Method column.

TABLE 1

| Ex. | | | | Meth- |
|---|---|---|---|---|
| # | Structure | Name | MS/NMR | od |
| 21 | 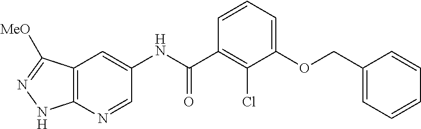 | 3-(benzyloxy)-2- chloro-N-(3- methoxy-1H- pyrazolo[3,4- b]pyridin-5- yl)benzamide | ¹H NMR (400 MHz, (CD₃)₂SO) δ 12.51 (s, 1H), 10.71 (s, 1H), 8.61 (s, 1H), 8.49 (s, 1H), 7.52-7.36 (m, 7H), 7.20-7.18 (d, 1H), 5.30 (s, 2H), 4.02 (s, 3H); m/z (APCI-pos) M + 1 = 409.1, 411.1 | 2 |
| 22 | 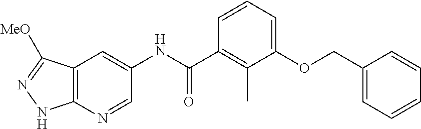 | 3-(benzyloxy)-N- (3-methoxy-1H- pyrazolo[3,4-b] yridine-5-yl)-2- methylbenzamide | ¹H NMR (400 MHz, (CD₃)₂SO) δ 12.51 (s, 1H), 10.51 (s, 1H), 8.67-8.66 (d, 1H), 8.50-8.49 (d, 1H), 7.50-7.48 (m, 2H), 7.40- 7.34 (m, 2H), 7.35-7.26 (m, 2H), 7.19-7.17 (d, 1H), 7.09-7.08 (d, 1H), 5.20 (s, 2H), 4.01 (s, 3H), 2.27 (s, 3H); m/z (APCI-pos) M + 1 = 389.2 | 2 |
| 23 | 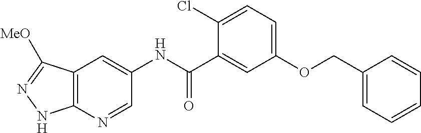 | 5-(benzyloxy)-2- chloro-N-(3- methoxy-1H- pyrazolo[3,4- b]pyridin-5- yl)benzamide | ¹H NMR (400 MHz, (CD₃)₂SO) δ 12.54 (s, 1H), 10.75 (s, 1H), 8.64 (s, 1H), 8.49 (s, 1H), 7.50-7.33 (m, 7H), 7.19-7.16 (m, 1H), 5.18 (s, 2H), 4.011 (s, 3H); m/z (APCI-pos) M + 1 = 409.2, 411.1 | 2 |
| 24 | 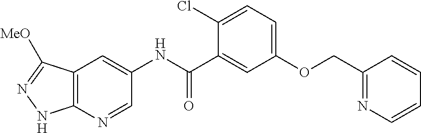 | 2-chloro-N-(3- methoxy-1H- pyrazolo[3,4- b]pyridin-5-yl)-5- (pyridin-2- ylmethoxy) benzamide | ¹H NMR (400 MHz, (CD₃)₂SO) δ 12.56 (s, 1H), 10.73 (s, 1H), 8.64 (s, 1H), 8.59 (s, 1H), 8.49 (s, 1H), 7.88-7.84 (t, 1H), 7.56-7.49 (m, 2H), 7.40-7.33 (m, 2H), 7.21-7.18 (d, 1H), 5.26 (s, 2H), 4.02 (s, 3H); m/z (APCI-pos) M + 1 = 410.1, 412.0 | 2 |
| 25 | 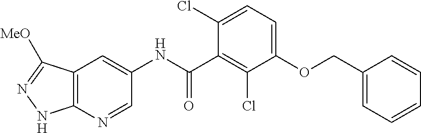 | 3-(benzyloxy)- 2,6-dichloro-N- (3-methoxy-1H- pyrazolo[3,4- b]pyridin-5- yl)benzamide | ¹H NMR (400 MHz, (CD₃)₂SO) δ 12.60 (s, 1H), 10.97 (s, 1H), 8.59 (s, 1H), 8.47 (s, 1H), 7.53-7.37 (m, 7H), 5.25 (s, 2H), 4.02 (s, 3H); m/z (APCI-pos) M + 1 = 443.1, 445.1 | 2 |
| 26 | 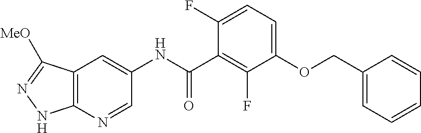 | 3-(benzyloxy)- 2,6-difluoro-N- (3-methoxy-1H- pyrazolo[3,4- b]pyridin-5- yl)benzamide | ¹H NMR (400 MHz, (CD₃)₂SO) δ 12.59 (s, 1H), 11.04 (s, 1H), 8.60-8.59 (d, 1H), 8.40-8.48 (d, 1H), 7.49-7.36 (m, 6H), 7.22- 7.17 (t, 1H), 5.24 (s, 2H), 4.01 (s, 3H); m/z (APCI- pos) M + 1 = 411.1 | 2 |
| 27 | 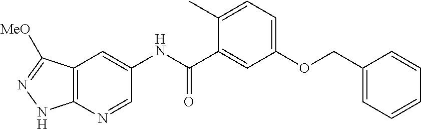 | 5-(benzyloxy)-N- (3-methoxy-1H- pyrazolo[3,4- b]pyridin-5-yl)-2- methylbenzamide | ¹H NMR (400 MHz, (CD₃)₂SO) δ 12.51 (s, 1H), 10.48 (s, 1H), 8.68-8.67 (d, 1H), 8.50-8.49 (d, 1H), 7.47-7.34 (m, 5H), 7.24- 7.19 (m, 2H), 7.08-7.05 (m, 1H), 5.15 (s, 2H), 4.01 (s, 3H), 2.33 (s, 3H); m/z (APCI-pos) M + 1 = 389.2 | 2 |
| 28 | 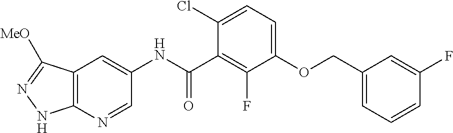 | 6-chloro-2- fluoro-3-(3- fluorobenzyloxy)- N-(3-methoxy- 1H-pyrazolo[3,4- b]pyridin-5- yl)benzamide | ¹H NMR (400 MHz, (CD₃)₂SO) δ 12.60 (s, 1H), 11.03 (s, 1H), 8.59-8.58 (d, 1H), 8.48-8.47 (d, 1H), 7.50-7.37 (m, 3H), 7.33- 7.29 (m, 2H), 7.22-7.17 (m, 1H), 5.30 (s, 2H), 4.02 (s, 3H); m/z (APCI-pos) M + 1 = 445.1, 447.1 | 3 |
| 29 | 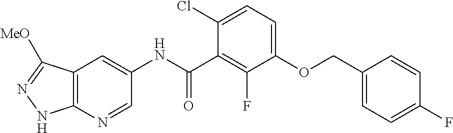 | 6-chloro-2- fluoro-3-(4- fluorobenzyloxy)- N-(3-methoxy- 1H-pyrazolo[3,4- b]pyridin-5- yl)benzamide | ¹H NMR (400 MHz, (CD₃)₂SO) δ 12.59 (s, 1H), 11.02 (s, 1H), 8.59-8.58 (d, 1H), 8.47-8.46 (d, 1H), 7.55-7.51 (m, 2H), 7.44- 7.37 (m, 2H), 7.28-7.23 (m, 2H), 5.25 (s, 2H), 4.01 (s, 3H); m/z (APCI-pos) M + 1 = 445.1, 447.1 | 3 |
| 30 | 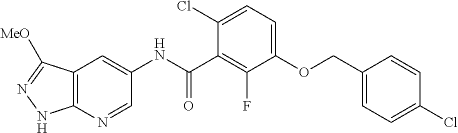 | 6-chloro-3-(4- chlorobenzyloxy)- 2-fluoro-N-(3- methoxy-1H- pyrazolo[3,4- b]pyridin-5- yl)benzamide | ¹H NMR (400 MHz, CD₃OD) δ 8.59-8.58 (d, 1H), 8.50-8.49 (d, 1H), 7.47-7.44 (m, 2H), 7.41- 7.38 (m, 2H), 7.28-7.24 (m, 2H), 5.20 (s, 2H), 4.09 (s, 3H); m/z (APCI-pos) M + 1 = 461.1, 463.0 | 3 |
| 31 | 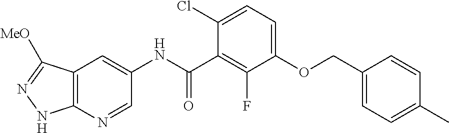 | 6-chloro-2- fluoro-N-(3- methoxy-1H- pyrazolo[3,4- b]pyridin-5-yl)-3- (4-methyl- benzyloxy) benzamide | ¹H NMR (400 MHz, CD₃OD) δ 8.59-8.58 (d, 1H), 8.50-8.49 (d, 1H), 7.34-7.32 (m, 2H), 7.28- 7.18 (m, 4H), 5.16 (s, 2H), 4.08 (s, 3H), 2.34 (s, 3H); m/z (APCI-pos) M + 1 = 441.1, 443.1 | 3 |
| 32 | 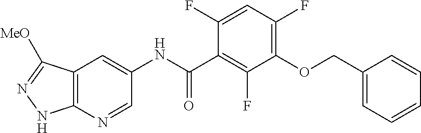 | 3-(benzyloxy)- 2,4,6-trifluoro-N- (3-methoxy-1H- pyrazolo[3,4- b]pyridin-5- yl)benzamide | ¹H NMR (400 MHz, CD₃OD) δ 8.58-8.57 (d, 1H), 8.50-8.49 (d, 1H), 7.46-7.43 (m, 2H), 7.39- 7.34 (m, 3H), 7.08-7.03 (m, 1H), 5.17 (s, 2H), 4.08 (s, 3H); m/z (APCI-pos) M + 1 = 429.1 | 3 |
| 33 | 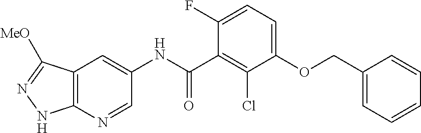 | 3-(benzyloxy)-2- chloro-6-fluoro- N-(3-methoxy- 1H-pyrazolo[3,4- b]pyridin-5- yl)benzamide | ¹H NMR (400 MHz, (CD₃)₂SO) δ 12.59 (s, 1H), 11.01 (s, 1H), 8.60-8.59 (d, 1H), 8.48-8.47 (d, 1H), 7.50-7.48 (m, 2H), 7.44- 7.34 (m, 5H), 5.27 (s, 2H), 4.01 (s, 3H); m/z (APCI- pos) M + 1 = 427.1, 429.1 | 4 |
| 34 | 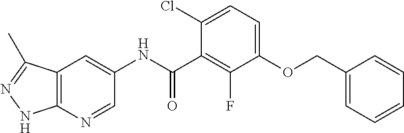 | 3-(benzyloxy)-6- chloro-2-fluoro- N-(3-methyl-1H- pyrazolo[3,4- b]pyridin-5- yl)benzamide | ¹H NMR (400 MHz, CD₃OD) δ 8.63-8.62 (d, 1H), 8.61-8.60 (d, 1H), 7.47-7.45 (m, 2H), 7.41- 7.24 (m, 5H), 5.22 (s, 2H), 2.58 (s, 3H); m/z (APCI- pos) M + 1 = 411.1, 413.1 | 6 |
| 35 | 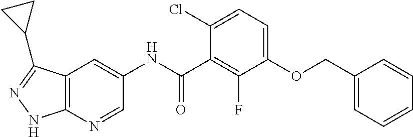 | 3-(benzyloxy)-6- chloro-N-(3- cyclopropyl-1H- pyrazolo[3,4- b]pyridin-5-yl)-2- fluorobenzamide | ¹H NMR (400 MHz, CD₃OD) δ 8.68-8.67 (d, 1H), 8.59-8.58 (d, 1H), 7.47-7.45 (m, 2H), 7.41- 7.24 (m, 5H), 5.22 (s, 2H), 2.30-2.24 (m, 1H), 1.30- 1.28 (m, 2H), 1.08-1.05 (m, 2H); m/z (APCI-pos) M + 1 = 437.1, 439.2 | 6 |
| 36 | 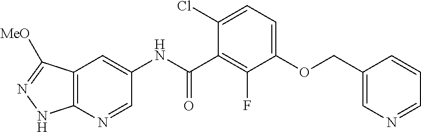 | 6-chloro-2- fluoro-N-(3- methoxy-1H- pyrazolo[3,4- b]pyridin-5-yl)-3- (pyridin-3- ylmethoxy) benzamide | ¹H NMR (400 MHz, (CD₃)₂SO) δ 12.60 (s, 1H), 11.03 (s, 1H), 8.70-8.44 (m, 6H), 7.48-7.40 (m, 2H), 5.32 (s, 2H), 4.01 (s, 3H); m/z (APCI-pos) M + 1 = 428.1, 430.1 | 19 |
| 37 | 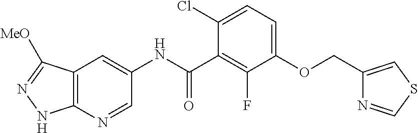 | 6-chloro-2- fluoro-N-(3- methoxy-1H- pyrazolo[3,4- b]pyridin-5-yl)-3- (thiazol-4- ylmethoxy) benzamide | ¹H NMR (400 MHz, (CD₃)₂SO) δ 12.60 (s, 1H), 11.03 (s, 1H), 9.15-9.14 (d, 1H), 8.59-8.58 (d, 1H), 8.47-8.46 (d, 1H), 7.87-7.87 (d, 1H), 7.54-7.49 (t, 1H), 7.40-7.38 (m, 1H), 5.39 (s, 2H), 4.01 (s, 3H); m/z (APCI-pos) M + 1 = 434.1, 436.1 | 19 |
| 38 | 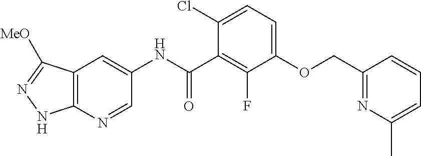 | 6-chloro-2- fluoro-N-(3- methoxy-1H- pyrazolo[3,4- b]pyridin-5-yl)-3- ((6- methylpyridin-2- yl)methoxy) benzamide | ¹H NMR (400 MHz, (CD₃)₂SO) δ 12.56 (br s, 1H), 11.01 (br s, 1H), 8.56 (s, 1H), 8.45 (s, 1H), 7.73 (m, 1H), 7.37-7.20 (m, 4H), 5.26 (s, 2H), 3.99 (s, 3H), 2.47 (s, 3H); m/z (APCI- POS) M + 1 = 442.1, 444.1 | 19 |
| 39 | 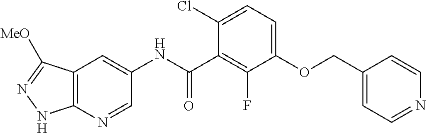 | 6-chloro-2- fluoro-N-(3- methoxy-1H- pyrazolo[3,4- b]pyridin-5-yl)-3- (pyridin-4- ylmethoxy) benzamide | ¹H NMR (400 MHz, CD₃OD) δ 12.56 (br s, 1H), 11.01 (br s, 1H), 8.58 (m, 3H), 8.46 (m, 1H), 7.43- 7.29 (m, 4H), 5.33 (s, 2H), 3.99 (s, 3H); m/z (APCI- POS) M + 1 = 428.2, 430.2 | 19 |
| 40 | 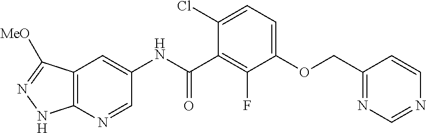 | 6-chloro-2- fluoro-N-(3- methoxy-1H- pyrazolo[3,4- b]pyridin-5-yl)-3- (pyrimidin-4- ylmethoxy) benzamide | ¹H NMR (400 MHz, (CD₃)₂SO) δ 9.14 (s, 1H), 8.81 (d, J = 5.7 Hz, 1H), 8.58 (s, 1H), 8.50 (s, 1H), 7.72 (d, J = 5.4 Hz, 1H), 7.29 (m, 2H), 5.33 (s, 2H), 4.08 (s, 3H); m/z (APCI-POS) M + 1 = 429.2, 431.2 | 19 |
| 41 | 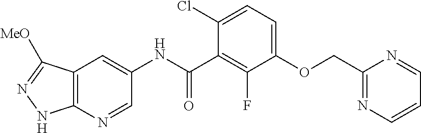 | 6-chloro-2- fluoro-N-(3- methoxy-1H- pyrazolo[3,4- b]pyridin-5-yl)-3- (pyrimidin-2- ylmethoxy) benzamide | ¹H NMR (400 MHz, (CD₃)₂SO) δ 12.56 (br s, 1H), 11.03 (br s, 1H), 8.82 (d, J = 5.4 Hz, 2H), 8.57 (s, 1H), 8.45 (s, 1H), 7.47 (t, J = 4.8 Hz, 1H), 7.26 (m, 2H), 5.45 (s, 2H), 3.99 (s, 3H); m/z (APCI-POS M + 1 = 429.1, 431.1 | 19 |
| 42 | 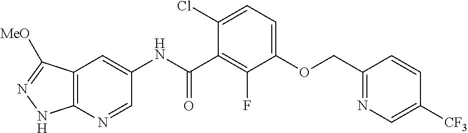 | 6-chloro-2- fluoro-N-(3- methoxy-1H- pyrazolo[3,4- b]pyridin-5-yl)-3- ((5- (trifluoromethyl) pyridin-2- yl)methoxy) benzamide | ¹H NMR (400 MHz, (CD₃)₂SO) δ 12.60 (br s, 1H), 11.05 (br s, 1H), 9.02 (s, 1H), 8.50 (s, 1H), 8.48 (s, 1H), 8.30 (d, J = 8.7 Hz, 1H), 7.77 (d, J = 8.4 Hz, 1H), 7.40 (m, 2H), 5.48 (s, 2H), 4.02 (s, 3H); m/z (APCI- POS) M + 1 = 496.1, 498.1 | 19 |
| 43 | 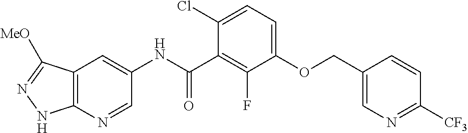 | 6-chloro-2- fluoro-N-(3- methoxy-1H- pyrazolo[3,4- b]pyridin-5-yl)-3- ((6- (trifluoromethyl) pyridin-3- yl)methoxy) benzamide | ¹H NMR (400 MHz, (CD₃)₂SO) δ 12.60 (br s, 1H), 11.04 (br s, 1H), 8.89 (s, 1H), 8.59 (s, 1H), 8.47 (s, 1H), 8.19 (d, J = 8.4 Hz, 1H), 7.99 (d, J = 7.8 Hz, 1H), 7.43 (m, 2H), 5.46 (s, 2H), 4.02 (s, 3H); m/z (APCI- POS) M + 1 = 496.1, 498.1 | 19 |
| 44 | 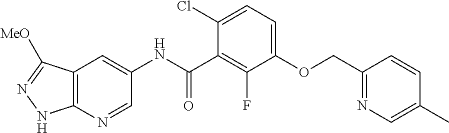 | 6-chloro-2- fluoro-N-(3- methoxy-1H- pyrazolo[3,4- b]pyridin-5-yl)-3- ((5- methylpyridin-2- yl)methoxy) benzamide | ¹H NMR (400 MHz, (CD₃)₂SO) δ 8.58 (s, 1H), 8.48 (s, 1H), 8.39 (s, 1H), 7.71 (d, J = 8.4 Hz, 1H), 7.51 (d, J = 7.8 Hz, 1H), 7.26 (m, 2H), 5.24 (s, 2H), 4.07 (s, 3H), 2.36 (s, 3H); m/z (APCI-POS) M + 1 = 442.1, 444.1 | 19 |
| 45 | 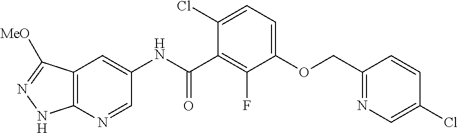 | 6-chloro-3-((5- chloropyridin-2- yl)methoxy)-2- fluoro-N-(3- methoxy-1H- pyrazolo[3,4- b]pyridin-5- yl)benzamide | ¹H NMR (400 MHz, (CD₃)₂SO) δ 12.59 (br s, 1H), 11.04 (br s, 1H), 8.66 (s, 1H), 8.59 (s, 1H), 8.48 (s, 1H), 8.02 (d, J = 8.4 Hz, 1H), 7.59 (d, J = 8.5 Hz, 1H), 7.39 (m, 2H), 5.36 (s, 2H), 4.01 (s, 3H); m/z (APCI- POS) M + 1 = 462.1, 464.1 | 19 |
| 46 | 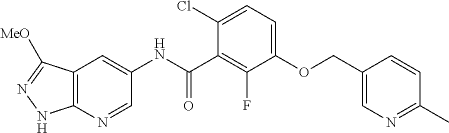 | 6-chloro-2- fluoro-N-(3- methoxy-1H- pyrazolo[3,4- b]pyridin-5-yl)-3- ((6- methylpyridin-3- yl)methoxy) benzamide | ¹H NMR (400 MHz, CD₃OD) δ 8.58 (s, 1H), 8.49 (m, 2H), 7.85 (d, J = 10.0 Hz, 1H), 7.36-7.27 (m, 3H), 5.24 (s, 2H), 4.08 (s, 3H), 2.55 (s, 3H); m/z (APCI-POS) M + 1 = 442.2, 444.2 | 19 |
| 47 | 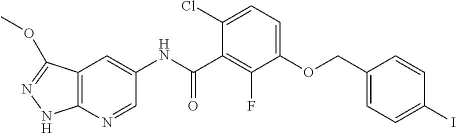 | 6-chloro-2- fluoro-3-(4- iodobenzyloxy)- N-(3-methoxy- 1H-pyrazolo[3,4- b]pyridin-5- yl)benzamide | ¹H NMR (400 MHz, CD₃OD) δ 8.59 (d, 1H), 8.50 (d, 1H), 7.53-7.57 (m, 3H), 7.23-7.26 (m, 3H), 5.17 (s, 2H), 4.08 (s, 3H); m/z (APCI-pos) M + 1 = 553.0, 555.1 | 19 |
| 48 | 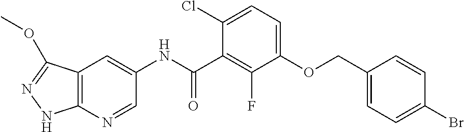 | 3-(4- bromobenzyloxy)- 6-chloro-2- fluoro-N-(3- methoxy-1H- pyrazolo[3,4- b]pyridin-5- yl)benzamide | m/z (APCI-pos) M + 1 = 506.9, 508.8 | 19 |
| 49 | 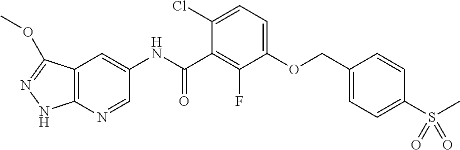 | 6-chloro-2- fluoro-N-(3- methoxy-1H- pyrazolo[3,4- b]pyridin-5-yl)-3- (4- methylsulfonyl) benzyloxy) benzamide | m/z (APCI-pos) M + 1 = 504.9, 506.9 | 19 |
| 50 | 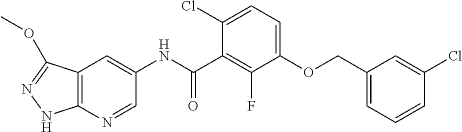 | 6-chloro-3-(3- chlorobenzyloxy)- 2-fluoro-N-(3- methoxy-1H- pyrazolo[3,4- b]pyridin-5- yl)benzamide | m/z (APCI-pos) M + 1 = 461.0, 463.1 | 19 |
| 51 | 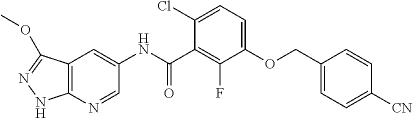 | 6-chloro-3-(4- cyanobenzyloxy)- 2-fluoro-N-(3- methoxy-1H- pyrazolo[3,4- b]pyridin-5- yl)benzamide | m/z (APCI-pos) M + 1 = 451.8, 453.8 | 19 |
| 52 | 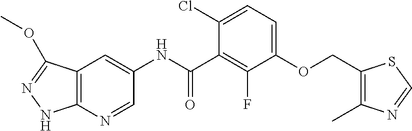 | 6-chloro-2- fluoro-N-(3- methoxy-1H- pyrazolo[3,4- b]pyridin-5-yl)-3- ((4- methylthiazol-5- yl)methoxy) benzamide | m/z (APCI-pos) M + 1 = 447.8, 449.9 | 19 |
| 53 | 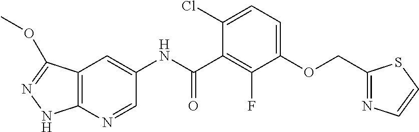 | 6-chloro-2- fluoro-N-(3- methoxy-1H- pyrazolo[3,4- b]pyridin-5-yl)-3- (thiazol-2- ylmethoxy) benzamide | m/z (APCI-pos) M + 1 = 434.0, 436.0 | 19 |
| 54 | 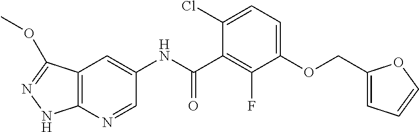 | 6-chloro-2- fluoro-3-(furan-2- ylmethoxy)-N-(3- methoxy-1H- pyrazolo[3,4- b]pyridin-5- yl)benzamide | m/z (APCI-pos) M + 1 = 417.0, 419.1 | 19 |
| 55 | 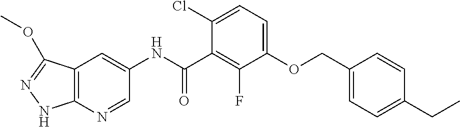 | 6-chloro-3-(4- ethylbenzyloxy)- 2-fluoro-N-(3- methoxy-1H- pyrazolo[3,4- b]pyridin-5- yl)benzamide | m/z (APCI-pos) M + 1 = 455.2, 457.2 | 19 |
| 56 | 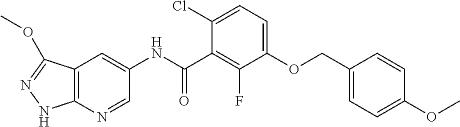 | 6-chloro-2- fluoro-N-(3- methoxy-1H- pyrazolo[3,4- b]pyridin-5-yl)-3- (4-methoxy- benzyloxy) benzamide | m/z (APCI-pos) M + 1 = 457.1, 459.0 | 19 |
| 57 | 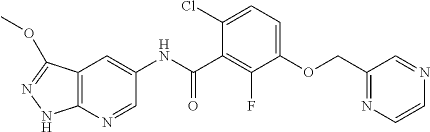 | 6-chloro-2- fluoro-N-(3- methoxy-1H- pyrazolo[3,4- b]pyridin-5-yl)-3- (pyrazin-2- ylmethoxy) benzamide | m/z (APCI-pos) M + 1 = 429.0, 431.1 | 19 |
| 58 | 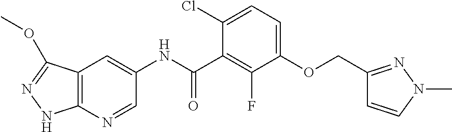 | 6-chloro-2- fluoro-N-(3- methoxy-1H- pyrazolo[3,4- b]pyridin-5-yl)-3- ((1-methyl-1H- pyrazolo-3- yl)methoxy) benzamide | m/z (APCI-pos) M + 1 = 431.1, 433.2 | 19 |
| 59 | 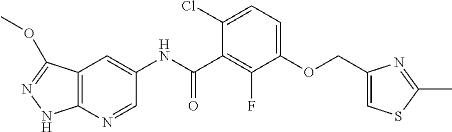 | 6-chloro-2- fluoro-N-(3- methoxy-1H- pyrazolo[3,4- b]pyridin-5-yl)-3- ((2- methylthiazol-4- yl)methoxy) benzamide | m/z (APCI-pos) M + 1 = 448.0, 450.1 | 19 |
| 60 | 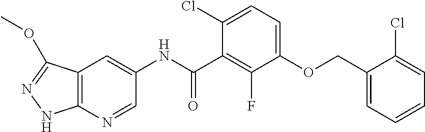 | 6-chloro-3-(2- chlorobenzyloxy)- 2-fluoro-N-(3- methoxy-1H- pyrazolo[3,4- b]pyridin-5- yl)benzamide | m/z (APCI-pos) M + 1 = 461.0, 463.0 | 19 |
| 61 | 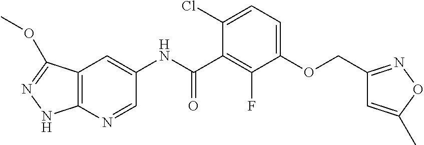 | 6-chloro-2- fluoro-N-(3- methoxy-1H- pyrazolo[3,4- b]pyridin-5-yl)-3- ((5- methylisoxazol-3- yl)methoxy) benzamide | m/z (APCI-pos) M + 1 = 431.1, 433.1 | 19 |
| 62 | 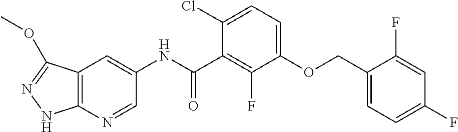 | 6-chloro-3-(2,4- difluorobenzyloxy)- 2-fluoro-N-(3- methoxy-1H- pyrazolo[3,4- b]pyridin-5- yl)benzamide | m/z (APCI-pos) M + 1 = 463.0, 465.1 | 19 |
| 63 | 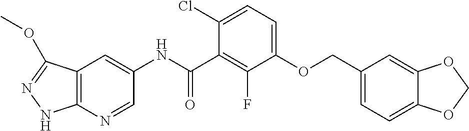 | 3- (benzo[d][1,3] dioxol-5- ylmethoxy)-6- chloro-2-fluoro- N-(3-methoxy- 1H-pyrazolo[3,4- b]pyridin-5- yl)benzamide | m/z (APCI-pos) M + 1 = 471.1, 473.1 | 19 |
| 64 | 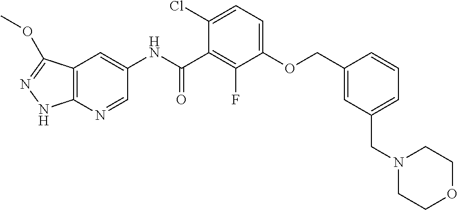 | 6-chloro-2- fluoro-N-(3- methoxy-1H- pyrazolo[3,4- b]pyridin-5-yl)-3- (3- (morpholinomethyl) benzyloxy) benzamide | m/z (APCI-pos) M + 1 = 526.2, 528.2 | 19 |
| 65 | 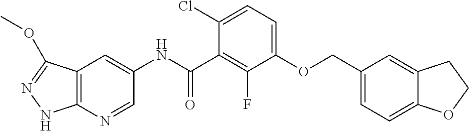 | 6-chloro-2-((2,3- dihydrobenzofuran- 5-yl)methoxy)- 2-fluoro-N-(3- methoxy-1H- pyrazolo[3,4- b]pyridin-5- yl)benzamide | m/z (APCI-pos) M + 1 = 468.9, 470.8 | 19 |
| 66 | 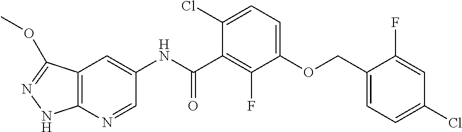 | 6-chloro-3-(4- chloro-2- fluorobenzyloxy)- 2-fluoro-N-(3- methoxy-1H- pyrazolo[3,4- b]pyridin-5- yl)benzamide | m/z (APCI-pos) M + 1 = 478.8, 480.9 | 19 |
| 67 | 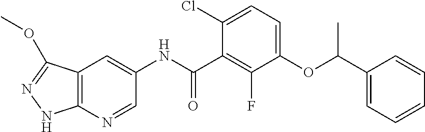 | 6-chloro-2- fluoro-N-(3- methoxy-1H- pyrazolo[3,4- b]pyridin-5-yl)-3- (1- phenylethoxy) benzamide | m/z (APCI-pos) M + 1 = 440.9, 442.9 | 19 |
| 68 | 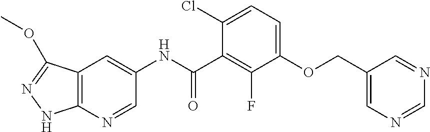 | 6-chloro-2- fluoro-N-(3- methoxy-1H- pyrazolo[3,4- b]pyridin-5-yl)-3- (pyrimidin-5- ylmethoxy) benzamide | m/z (APCI-pos) M + 1 = 429.1, 431.0 | 19 |
| 69 | 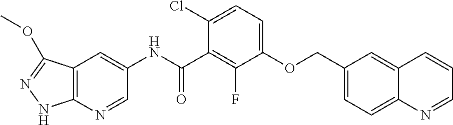 | 6-chloro-2- fluoro-N-(3- methoxy-1H- pyrazolo[3,4- b]pyridin-5-yl)-3- (quinolin-6- ylmethoxy) benzamide | m/z (APCI-pos) M + 1 = 478.1, 480.1 | 19 |
| 70 | 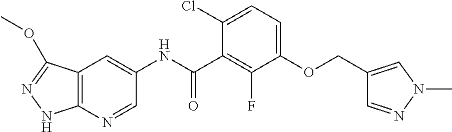 | 6-chloro-2- fluoro-N-(3- methoxy-1H- pyrazolo[3,4- b]pyridin-5-yl)-3- ((1-methyl-1H- pyrazol-4- yl)methoxy) benzamide | m/z (APCI-pos) M + 1 = 431.1, 433.1 | 19 |
| 71 | 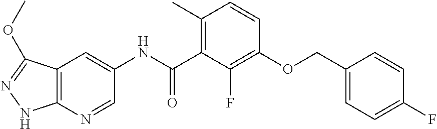 | 2-fluoro-3-(4- fluorobenzyloxy)- N-(3-methoxy- 1H-pyrazolo[3,4- b]pyridin-5-yl)-6- methylbenzamide | ¹H NMR (400 MHz, (CD₃)₂SO) δ 12.55 (s, 1H), 10.81 (s, 1H), 8.62-8.61 (d, 1H), 8.50-8.49 (d, 1H), 7.54-7.50 (m, 2H), 7.28- 7.22 (m, 3H), 7.08-7.06 (m, 1H), 5.19 (s, 2H), 4.01 (s, 3H), 2.26 (s, 3H); m/z (APCI-pos) M + 1 = 425.1 | 5 |
| 72 | 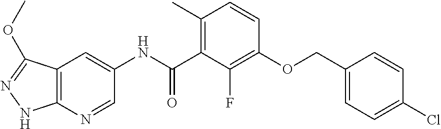 | 3-(4- chlorobenzyloxy)- 2-fluoro-N-(3- methoxy-1H- pyrazolo[3,4- b]pyridin-5-yl)-6- methylbenzamide | ¹H NMR (400 MHz, (CD₃)₂SO) δ 12.55 (s, 1H), 10.81 (s, 1H), 8.62-8.61 (d, 1H), 8.50-8.49 (d, 1H), 7.48 (s, 4H), 7.26-7.22 (t, 1H), 7.08-7.05 (d, 1H), 5.22 (s, 2H), 4.01 (s, 3H), 2.26 (s, 3H); m/z (APCI-pos) M + 1 = 441.1, 443.1 | 5 |
| 73 | 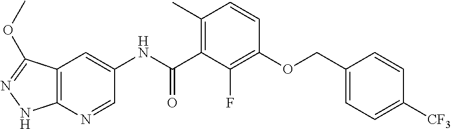 | 2-fluoro-N-(3- methoxy-1H- pyrazolo[3,4- b]pyridin-5-yl)-6- methyl-3-(4- (trifluoromethyl) benzyloxy) benzamide | ¹H NMR (400 MHz, (CD₃)₂SO) δ 12.56 (s, 1H), 10.82 (s, 1H), 8.62-8.62 (d, 1H), 8.50-8.49 (d, 1H), 7.80-7.78 (d, 2H), 7.70-7.68 (d, 2H), 7.27-7.22 (t, 1H), 7.08-7.06 (d, 1H), 5.34 (s, 2H), 4.01 (s, 3H), 2.26 (s, 3H); m/z (APCI-pos) M + 1 = 475.1 | 5 |
| 74 | 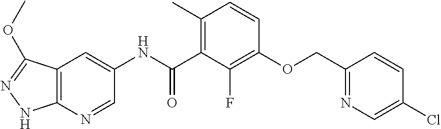 | 3-((5- chloropyridin-2- yl)methoxy)-2- fluoro-N-(3- methoxy-1H- pyrazolo[3,4- b]pyridin-5-yl)-6- methylbenzamide | ¹H NMR (400 MHz, (CD₃)₂SO) δ 12.56 (s, 1H), 10.83 (s, 1H), 8.66-8.65 (d, 1H), 8.62-8.61 (d, 1H), 8.51-8.50 (d, 1H), 8.03-8.00 (m, 1H), 7.60-7.57 (d, 1H), 7.26-7.22 (t, 1H), 7.08-7.06 (d, 1H), 5.30 (s, 2H), 4.01 (s, 3H), 2.26 (s, 3H); m/z (APCI-pos) M + 1 = 442.1, 443.1 | 5 |
| 75 | 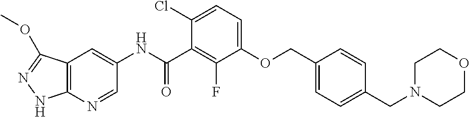 | 6-chloro-2- fluoro-N-(3- methoxy-1H- pyrazolo[3,4- b]pyridin-5-yl)-3-(4- (morpholinomethyl) benzyloxy)benzamide | m/z (APCI-pos) M + 1 = 526.0, 528.0 | 19 |
| 76 | 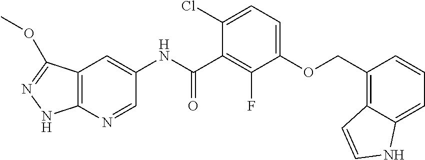 | 3-((1H-indol-4- yl)methoxy)-6- chloro-2-fluoro- N-(3-methoxy- 1H-pyrazolo[3,4- b]pyridin-5- yl)benzamide | m/z (APCI-pos) M + 1 = 465.8, 467.7 | 19 |
| 77 | 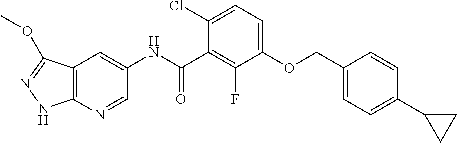 | 6-chloro-3-(4-cyclo- propylbenzyloxy)- 2-fluoro-N- (3-methoxy-1H- pyrazolo[3,4- b]pyridin-5- yl)benzamide | ¹H NMR (400 MHz, (CD₃)₂SO) δ 12.59 (s, 1H), 11.01 (s, 1H), 8.58 (s, 1H), 8.47 (s, 1H), 7.33-7.42 (m, 4H), 7.11 (d, 2H), 5.20 (s, 2H), 4.04 (s, 3H), 1.89-1.95 (m, 4H), 0.93-0.97 (m, 2H), 0.65-0.69 (m, 2H); m/z (APCI-pos) M + 1 = 467.1, 469.1 | 19 |

### Example 78

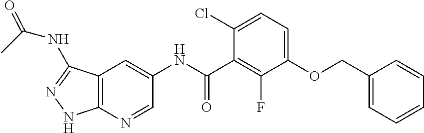

### N-(3-acetamido-1H-pyrazolo[3,4-b]pyridin-5-yl)-3-(benzyloxy)-6-chloro-2-fluorobenzamide

Step A: N-(5-Nitro-1H-pyrazolo[3,4-b]pyridin-3-yl)acetamide (74%) was prepared according to the general procedure in Example 1, Step A, substituting 1H-pyrazole-3,5-diamine (US 2007/0082902) for 3-methoxy-1H-pyrazol-5-amine and acetic acid for water.

Step B: N-(5-Amino-1H-pyrazolo[3,4-b]pyridin-3-yl)acetamide (96%) was prepared according to Example 1, Step B, substituting N-(5-nitro-1H-pyrazolo[3,4-b]pyridin-3-yl)acetamide for 3-methoxy-5-nitro-1H-pyrazolo[3,4-b]pyridine.

Step C: N-(3-Acetamido-1H-pyrazolo[3,4-b]pyridin-5-yl)-3-(benzyloxy)-6-chloro-2-fluorobenzamide (61%) was prepared according to the general procedure in Example 1, Step C substituting N-(5-amino-1H-pyrazolo[3,4-b]pyridin-3-yl)acetamide for 3-methoxy-1H-pyrazolo[3,4-b]pyridin-5-amine and 3-(benzyloxy)-6-chloro-2-fluorobenzoic acid for 3-(benzyloxy)benzoic acid. ¹H NMR (400 MHz, (CD₃)₂SO) δ 13.22 (s, 1H), 10.99 (s, 1H), 10.63 (s, 1H), 8.72 (s, 1H), 8.64 (s, 1H), 7.36-7.49 (m, 7H), 5.27 (s, 2H), 2.12 (s, 3H); m/z (APCI-pos) M+1=452.2, 454.2.

It will be understood that the enumerated embodiments are not intended to limit the invention to those embodiments. On the contrary, the invention is intended to cover all alternatives, modifications and equivalents, which may be included within the scope of the present invention as defined by the claims. Thus, the foregoing description is considered as illustrative only of the principles of the invention.

The words “comprise,” “comprising,” “include,” “including,” and “includes” when used in this specification and in the following claims are intended to specify the presence of stated features, integers, components, or steps, but they do not preclude the presence or addition of one or more other features, integers, components, steps, or groups thereof.

What is claimed is:

1. A compound selected from Formula I: 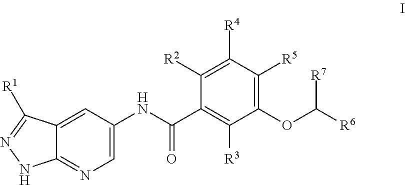
and stereoisomers, tautomers and pharmaceutically acceptable salts thereof, wherein:
R¹ is selected from:
hydrogen,
halogen,
CN,
NR^{a}R^{b},
OR^{C},
SR^{d},
phenyl optionally substituted with one to three R^{e} groups, a 5-6 membered heteroaryl optionally substituted with C₁-C₄ alkyl,
a saturated or partially unsaturated C₃-C₆ cycloalkyl optionally substituted with halogen or C₁-C₄ alkyl,
a saturated or partially unsaturated 4-6 membered heterocyclyl optionally substituted with C₁-C₄ alkyl,
C₂-C₆ alkynyl optionally substituted with halogen, OR^{c} or NR^{a}R^{b},
C₂-C₆ alkenyl optionally substituted with halogen, OR^{c} or NR^{a}R^{b}, or
C₁-C₆ alkyl optionally substituted with one to three R^{f} groups;
R² and R³ are independently selected from hydrogen, halogen, C₁-C₃ alkyl and C₁-C₃ alkoxy;
R⁴ and R⁵ are independently selected from hydrogen, halogen or C₁-C₃ alkyl;
R⁶ is selected from phenyl, a 5-6 membered heteroaryl, a 9-10 membered bicyclic heterocyclyl or a 9-10 membered bicyclic heteroaryl, wherein the phenyl, heteroaryls and heterocyclyl are optionally substituted with one, two or three R^{g} groups;
R⁷ is hydrogen or methyl;
R^{a} and R^{b} are independently selected from hydrogen, phenyl and C₁-C₄ alkyl optionally substituted with oxo;
R^{c} is selected from a 4-6 membered heterocyclyl and C₁-C₆ alkyl optionally substituted with halogen, OH, OCH₃, C₃-C₆ cycloalkyl, a 4-6 membered heterocyclyl or NR^{a}R^{b};
R^{d} is C₁-C₆ alkyl;
each R^{e} is independently selected from halogen, CF₃, C₁-C₄ alkyl or C₁-C₄ alkoxy, wherein the alkyl or alkoxy are optionally substituted with OH, NR^{a}R^{b} or a 5-6 membered heterocyclyl optionally substituted with C₁-C₃ alkyl;
each R^{f} is independently selected from halogen, OH, OCH₃, oxo, NR^{a}R^{b}, or C₃-C₆ cycloalkyl; and
each R^{g} is selected from halogen, CN, SO₂CH₃, C₁-C₃ alkyl, C₁-C₃ alkoxy, or C₃-C₆ cycloalkyl, wherein the alkyl is optionally substituted with halogen or a 3-6 membered heterocyclyl.

2. A compound of claim 1, wherein R¹ is selected from NR^{a}R^{b}, OR^{c}, SR^{d}, a 5-6 membered heteroaryl, C₃-C₆ cycloalkyl and C₁-C₆ alkyl optionally substituted with one to three R^{f} groups.

3. A compound as claimed in claim 1 or 2, wherein R¹ is selected from methyl, ethyl, isopropyl, CF₃, —OCH₃, —OCH₂CH₃, —OCH(CH₃)₂, —OCH₂CH₂F, —OCH₂CH₂OH, —OCH₂CH₂OCH₃, —OCH₂CH₂N(CH₃)₂, —NHCH₃, —N(CH₃)₂, —NC(═O)CH₃, —SCH₃, cyclopropyl, 1-methyl-cyclopropyl and furany-2-yl.

4. A compound as claimed in any one of claims 1 to 3, wherein R⁶ is selected from phenyl, 2-fluorophenyl, 3-fluorophenyl, 4-fluorophenyl, 2-chlorophenyl, 3-chlorophenyl, 4-chlorophenyl, 4-bromophenyl, 4-iodophenyl, 2,4-difluorophenyl, 2-fluoro-4-chlorophenyl, 4-methylphenyl, 4-ethylphenyl, 4-trifluoromethylphenyl, 4-methoxyphenyl, 4-cyanophenyl, 4-(methylsulfonyl)phenyl, 3-(morpholinomethyl)phenyl, 4-(morpholinomethyl)phenyl, 4-cyclopropylphenyl, furan-2-yl, 1-methyl-1H-pyrazol-3-yl, 1-methyl-1H-pyrazol-4-yl, 5-methylisoxazol-3-yl, thiazol-2-yl, thiazol-4-yl, 2-methylthiazol-4-yl, 4-methylthiazol-5-yl, pyridin-2-yl, pyridin-3-yl, pyridin-4-yl, 5-chloropyridin-2-yl, 5-methylpyridin-2-yl, 6-methylpyridin-2-yl, 6-methylpyridin-3-yl, 5-(trifluoromethyl)pyridin-2-yl, 6-(trifluoromethyl)pyridin-3-yl, pyrimidin-2-yl, pyrimidin-4-yl, pyrimidin-5-yl, pyrazin-2-yl, 2,3-dihydrobenzofuran-5-yl, benzo[d][1,3]dioxol-5-yl, quinolin-6-yl and 1H-indol-4-yl.

5. A compound as claimed in any one of claims 1 to 4, wherein R², R³, R⁴ and R⁵ are independently selected from hydrogen, halogen and C₁-C₃ alkyl.

6. A compound as claimed in any one of claims 1 to 5, wherein the residue: 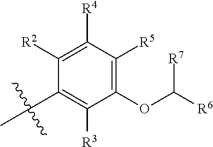
of Formula I, wherein the wavy line represents the point of attachment of the residue in Formula I, is selected from:

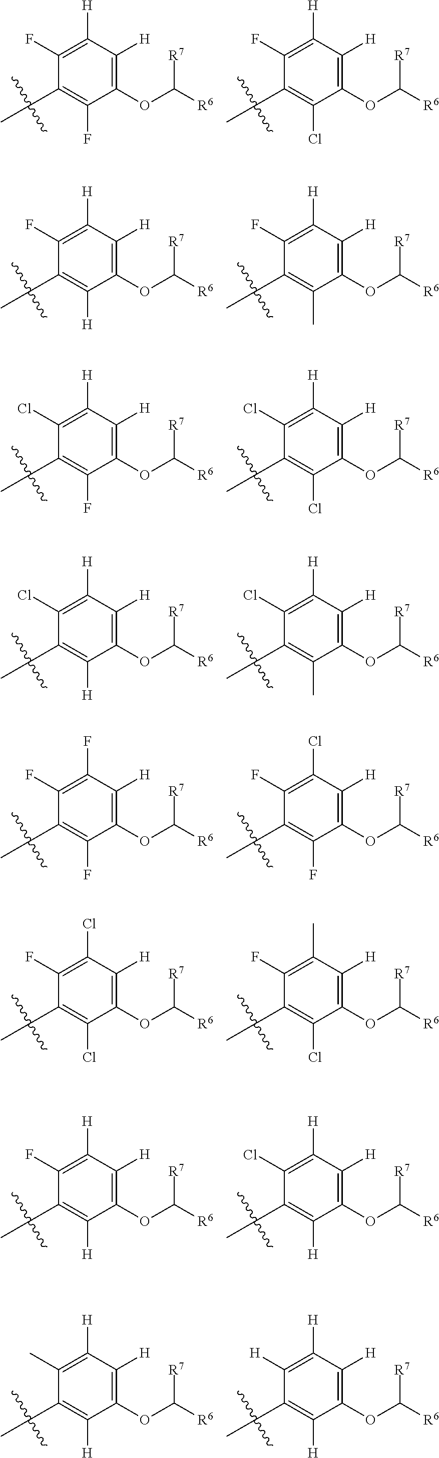

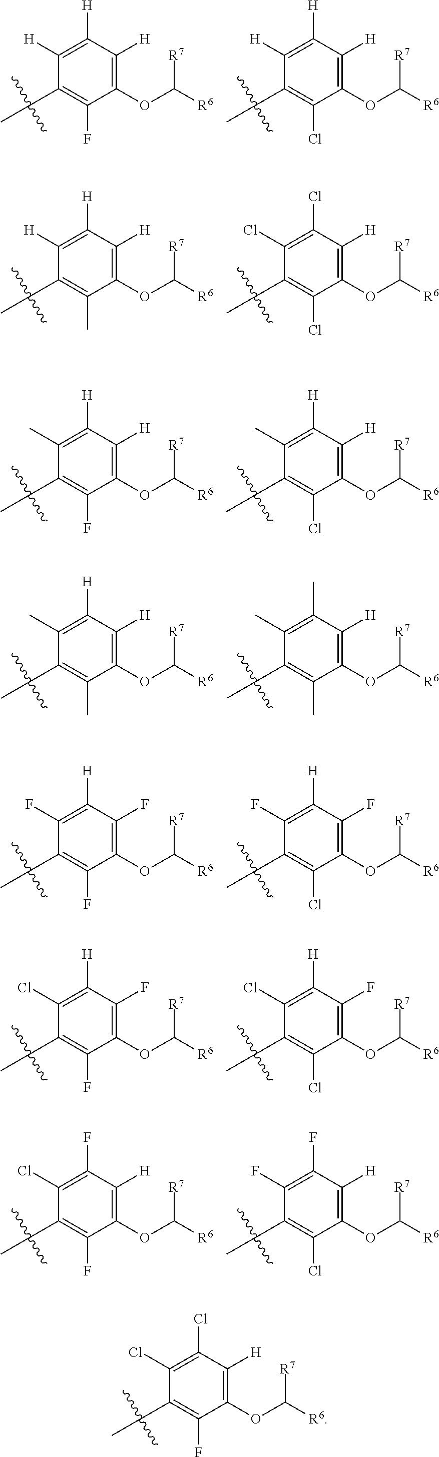

7. A compound as claimed in any one of claims 1 to 6, wherein R² and R⁴ are independently selected from hydrogen, halogen or methyl; R³ is Cl; and R⁵ is hydrogen or F.

8. A compound of Formula I as defined in claim 1 and named in any one of Examples 1 to 78 herein.

9. A pharmaceutical composition, comprising a compound as claimed in any one of claims 1 to 8, and a pharmaceutically acceptable carrier or excipient.

10. A method of preventing or treating a disease or disorder modulated by b-Raf, comprising administering to a mammal in need of such treatment an effective amount of a compound of any one of claims 1 to 8.

11. A method of preventing or treating cancer, comprising administering to a mammal in need of such treatment an effective amount of a compound of any one of claims 1 to 8, alone or in combination with one or more additional compounds having anti-cancer properties.

12. The method of claim 11, wherein the cancer is a sarcoma.

13. The method of claim 11, wherein the cancer is a carcinoma.

14. The method of claim 13, wherein the carcinoma is squamous cell carcinoma.

15. The method of claim 13, wherein the carcinoma is adenoma or adenocarcinoma.

16. The method of claim 11, wherein the cancer is breast, ovary, cervix, prostate, testis, genitourinary tract, esophagus, larynx, glioblastoma, neuroblastoma, stomach, skin, keratoacanthoma, lung, epidermoid carcinoma, large cell carcinoma, non-small cell lung carcinoma (NSCLC), small cell carcinoma, lung adenocarcinoma, bone, colon, adenoma, pancreas, adenocarcinoma, thyroid, follicular carcinoma, undifferentiated carcinoma, papillary carcinoma, seminoma, melanoma, sarcoma, bladder carcinoma, liver carcinoma and biliary passages, kidney carcinoma, myeloid disorders, lymphoid disorders, hairy cells, buccal cavity and pharynx (oral), lip, tongue, mouth, pharynx, small intestine, colon-rectum, large intestine, rectum, brain and central nervous system, Hodgkin's and leukemia.

17. A method of treating a hyperproliferative disease in a mammal comprising administering a therapeutically effective amount of a compound of any one of claims 1 to 8 to the mammal.

18. A compound as claimed in any one of claims 1 to 8 for use in therapy.

19. A compound as claimed in any one of claims 1 to 8 for use in the treatment of a hyperproliferative disease.

20. Use of a compound of any one of claims 1 to 8 in the manufacture of a medicament for the treatment of a hyperproliferative disease.

21. Use of a compound as claimed in any one of claims 1 to 8, in the manufacture of a medicament, for use as a b-Raf inhibitor in the treatment of a patient undergoing cancer therapy.

22. A method of preventing or treating kidney disease, comprising administering to a mammal in need of such treatment an effective amount of a compound of any one of claims 1 to 8, or a stereoisomer, tautomer or pharmaceutically acceptable salt thereof, alone or in combination with one or more additional compounds.

23. The method of claim 22, wherein the kidney disease is polycystic kidney disease.

24. A compound of any one of claims 1 to 8 for use in the treatment of a kidney disease.

25. The compound of claim 24, wherein the kidney disease is polycystic kidney disease.

26. Use of a compound of any one of claims 1 to 8 in the manufacture of a medicament for the treatment of a kidney disease.

27. The use of claim 26, wherein the kidney disease is polycystic kidney disease.

28. A pharmaceutical composition comprising a compound as claimed in any one of claims 1 to 8 for use in the treatment of a hyperproliferative disease.

29. A pharmaceutical composition comprising a compound as claimed in any one of claims 1 to 8 for use in the treatment of cancer.

30. A pharmaceutical composition comprising a compound as claimed in any one of claims 1 to 8 for use in the treatment of kidney disease.

31. The composition of claim 30, wherein the kidney disease is polycystic kidney disease.
